(19) United States
(12) Patent Application Publication
(10) Pub. No.: US 2026/0173641 A1
(43) Pub. Date: Jun. 18, 2026

(54) COMPOUND FOR ORGANIC OPTOELECTRONIC ELEMENT, COMPOSITION FOR SAME, ORGANIC OPTOELECTRONIC ELEMENT, AND DISPLAY DEVICE

(71) Applicant: SAMSUNG SDI CO., LTD., Yongin-si, Gyeonggi-do (KR)

(72) Inventors: Byoungkwan LEE, Suwon-si, Gyeonggi-do (KR); Dong Min KANG, Suwon-si, Gyeonggi-do (KR); Kipo JANG, Suwon-si, Gyeonggi-do (KR); Jiyun KWON, Suwon-si, Gyeonggi-do (KR); Hansol SEO, Suwon-si, Gyeonggi-do (KR); Jonghoon KIM, Suwon-si, Gyeonggi-do (KR); Yunsoo KIM, Suwon-si, Gyeonggi-do (KR); Sunghyun JUNG, Suwon-si, Gyeonggi-do (KR); Youngkyoung JO, Suwon-si, Gyeonggi-do (KR); Ho Kuk JUNG, Suwon-si, Gyeonggi-do (KR); Jongwoo CHOI, Suwon-si, Gyeonggi-do (KR)

(21) Appl. No.: 19/126,007

(22) PCT Filed: Nov. 7, 2023
(86) PCT No.: PCT/KR2023/017775

§ 371 Date: Apr. 30, 2025

(30) Foreign Application Priority Data
Nov. 7, 2022 (KR) 10-2022-0147200

(51) Int. Cl. H10K 50/12; C07B 59/00; H10K 85/60

(52) U.S. Cl. CPC H10K 50/12; C07B 59/004; H10K 85/622; H10K 85/626; H10K 85/633; H10K 85/636; H10K 85/654; H10K 85/6572; H10K 85/6574; H10K 85/6576; C07B 2200/05

(57) ABSTRACT

Provided are a compound for an organic optoelectronic device represented by Chemical Formula 1, a composition for an organic optoelectronic device including the same, an organic optoelectronic device, and a display device. The contents of Chemical Formula 1 are as defined in the specification.

### TECHNICAL FIELD

A compound for an organic optoelectronic device, a composition for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is a light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a compound for an organic optoelectronic device that realize a high-efficiency and long life-span organic optoelectronic device.

Another embodiment provides a composition for an organic optoelectronic device including the compound for an organic optoelectronic device.

Another embodiment provides an organic optoelectronic device including the compound for an organic optoelectronic device or composition for an organic optoelectronic device.

Another embodiment provides a display device including the organic optoelectronic device.

### Technical Solution

According to an embodiment, a compound for an organic optoelectronic device represented by Chemical Formula 1 is provided.

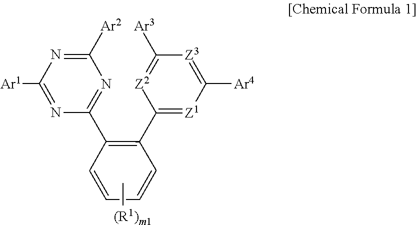

In Chemical Formula 1,
Z¹ to Z³ are each independently N or CR^{a},
at least two of Z¹ to Z³ are N,
Ar¹ to Ar⁴ are each independently hydrogen, deuterium, a cyano group, a halogen, a substituted or unsubstituted C6 to C30 aryl group, a substituted or unsubstituted carbazolyl group, a substituted or unsubstituted dibenzofuranyl group, a substituted or unsubstituted dibenzothiophenyl group, or a combination thereof, and
any one of Ar¹ to Ar⁴ is a group represented by Chemical Formula A,

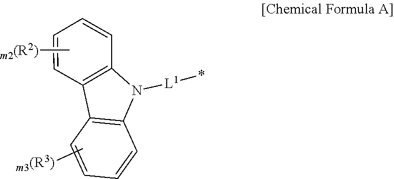

L¹ is a single bond or a substituted or unsubstituted C6 to C20 arylene group,
R^{a} and R¹ to R³ are each independently hydrogen, deuterium, a cyano group, a halogen, a substituted or unsubstituted C1 to C30 alkyl group, a substituted or unsubstituted C6 to C30 aryl group, or a substituted or unsubstituted C2 to C30 heterocyclic group,
m1 to m3 are each independently one of integers of 1 to 4,
* is a linking point, and
Chemical formula 1 satisfies at least one of the following conditions (i) to (iii):
   (i) at least one of the remaining substituents not substituted by Chemical Formula A among Ar¹ to Ar⁴ is a substituted or unsubstituted C10 to C30 aryl group;
   (ii) at least one of R² and R³ is a substituted or unsubstituted C10 to C30 aryl group; and
   (iii) R¹ is a substituted or unsubstituted C6 to C30 aryl group.

According to another embodiment, a composition for an organic optoelectronic device including a first compound and a second compound is provided.

The first compound may be the aforementioned compound for an organic optoelectronic device and the second compound may be represented by Chemical Formula Chemical Formula 2; or a combination of Chemical Formula 3 and Chemical Formula 4.

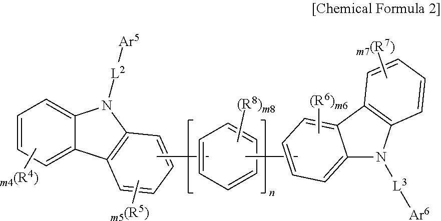

In Chemical Formula 2,
R⁴ to R⁸ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁵ and Ar⁶ are each independently a substituted or unsubstituted C6 to C20 aryl group or a substituted or unsubstituted C2 to C30 heterocyclic group,
L² and L³ are each independently a single bond or a substituted or unsubstituted C6 to C20 arylene group,
m4, m7, and m8 are each independently one of integers of 1 to 4,
m5 and m6 are each independently one of integers of 1 to 3, and
n is one of integers of 0 to 2;

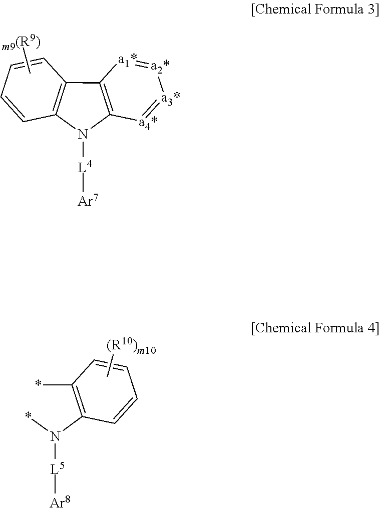

wherein, in Chemical Formula 3 and Chemical Formula 4,
a₁* to a₄* in Chemical Formula 3 are each independently linking carbon (C) or C-L^{a}-R^{a}
among a₁* to a₄* in Chemical Formula 3, two adjacent ones are each linked to *s in Chemical Formula 4,
L^{a}, L⁴, and L⁵ are each independently a single bond or a substituted or unsubstituted C6 to C20 arylene group,
R^{a}, R⁹, and R¹⁰ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁷ and Ar⁸ are each independently a substituted or unsubstituted C6 to C20 aryl group or a substituted or unsubstituted C2 to C30 heterocyclic group, and
m9 and m10 are each independently one of integers of 1 to 4.

According to another embodiment, an organic optoelectronic device includes an anode and a cathode facing each other, and at least one organic layer between the anode and the cathode, wherein the organic layer includes the aforementioned compound for an organic optoelectronic device or composition for an organic optoelectronic device.

According to another embodiment, a display device including the organic optoelectronic device is provided.

### Advantageous Effects

An organic optoelectronic device having high efficiency and a long life-span may be realized.

### DESCRIPTION OF THE DRAWINGS

FIG. 1 is a cross-sectional view illustrating an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### BEST MODE

Hereinafter, embodiments of the present invention are described in detail. However, these embodiments are exemplary, and this disclosure is not limited thereto.

In the present specification, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In a specific example of the present invention, the “substituted” refers to replacement of at least one hydrogen of a substituent or a compound by deuterium, a C1 to C20 alkyl group, a C6 to C30 aryl group, or a cyano group. In a specific example of the present invention, the “substituted” refers to replacement of at least one hydrogen of a substituent or a compound by deuterium, a C1 to C5 alkyl group, a C6 to C18 aryl group, or a cyano group. In a specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In the present specification, “unsubstituted” refers to non-replacement of a hydrogen atom by another substituent and remaining of the hydrogen atom.

In the present specification, “hydrogen substitution (-H)” may include “deuterium substitution (-D)” or “tritium substitution (-T).” In the present specification, when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quaterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

In the present specification, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

A compound for an organic optoelectronic device according to an embodiment is represented by Chemical Formula 1.

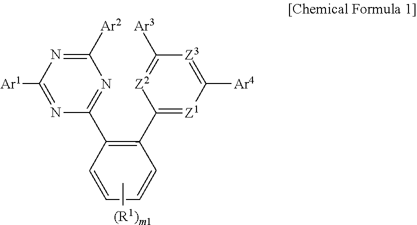

In Chemical Formula 1,
Z¹ to Z³ are each independently N or CR^{a},
at least two of Z¹ to Z³ are N,
Ar¹ to Ar⁴ are each independently hydrogen, deuterium, a cyano group, a halogen, a substituted or unsubstituted C6 to C30 aryl group, a substituted or unsubstituted carbazolyl group, a substituted or unsubstituted dibenzofuranyl group, a substituted or unsubstituted dibenzothiophenyl group, or a combination thereof,
any one of Ar¹ to Ar⁴ is a group represented by Chemical Formula A,

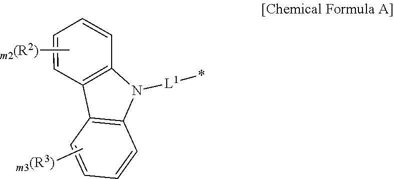

L¹ is a single bond or a substituted or unsubstituted C6 to C20 arylene group,
R^{a} and R¹ to R³ are each independently hydrogen, deuterium, a cyano group, a halogen, a substituted or unsubstituted C1 to C30 alkyl group, a substituted or unsubstituted C6 to C30 aryl group, or a substituted or unsubstituted C2 to C30 heterocyclic group,
m1 to m3 are each independently one of integers of 1 to 4,
* is a linking point, and
Chemical formula 1 satisfies at least one of the following conditions (i) to (iii).
   (i) at least one of the remaining substituents not substituted by Chemical Formula A among Ar¹ to Ar⁴ is a substituted or unsubstituted C10 to C30 aryl group;
   (ii) at least one of R² and R³ is a substituted or unsubstituted C10 to C30 aryl group; and
   (iii) R¹ is a substituted or unsubstituted C6 to C30 aryl group.

In Chemical Formula 1, when m1 is 2 or more, each R¹ may be the same or different from each other.

In Chemical Formula 1, when m2 is 2 or more, each R² may be the same or different from each other.

In Chemical Formula 1, when m3 is 2 or more, each R³ may be the same or different from each other.

The compound represented by Chemical Formula 1 is characterized in that a triazine is linked to another triazine or pyrimidine via ortho-phenylene, wherein the triazine and/or pyrimidine essentially include a carbazolyl group, and in particular, the triazine, pyrimidine (or triazine), the linking group (ortho-phenylene), and the carbazolyl group additionally includes an aryl group of C10 or more.

By linking another triazine or pyrimidine is linked to triazine via to ortho-phenylene, the life-span may be improved compared to other substitution positions, and especially, excellent life-span characteristics can be realized by essentially including the carbazolyl group. By additionally including an aryl group of C10 or more, the electron cloud of LUMO is expanded, and the electron transport ability is enhanced, thereby improving the driving voltage, and appropriately controlling the luminescent zone to achieve high efficiency.

For example, depending on the substitution position of the carbazolyl group, Chemical Formula 1 may be represented by Chemical Formula 1A or Chemical Formula 1B.

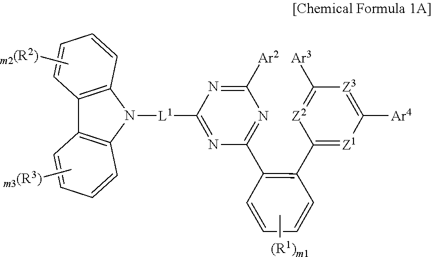

In Chemical Formula 1A, the definitions of Z¹ to Z³, Ar¹ to Ar⁴, L¹, R¹ to R³ and m1 to m3 are as defined in Chemical Formula 1,
Chemical Formula 1A satisfies at least one of the following conditions (iii) to (v):
   (iii) at least one of Ar² to Ar⁴ is a substituted or unsubstituted C10 to C30 aryl group;
   (iv) at least one of R² and R³ is a substituted or unsubstituted C10 to C30 aryl group; and
   (v) R¹ is a substituted or unsubstituted C6 to C30 aryl group.

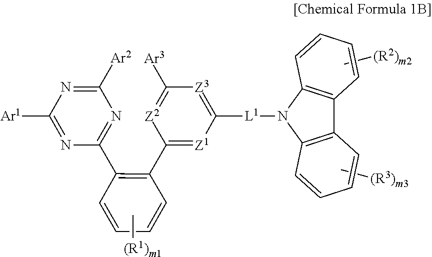

In Chemical Formula 1B, the definitions of Z¹ to Z³, Ar¹ to Ar⁴, L¹, R¹ to R³ and m1 to m3 are as defined in Chemical Formula 1,
Chemical Formula 1B satisfies at least one of the following conditions (vi) to (viii):
   (vi) at least one of Ar¹ to Ar³ is a substituted or unsubstituted C10 to C30 aryl group;
   (vii) at least one of R² and R³ is a substituted or unsubstituted C10 to C30 aryl group; and
   (viii) R¹ is a substituted or unsubstituted C6 to C30 aryl group.

As a specific example, Chemical Formula 1A may be represented by any one of Chemical Formula 1A-I, Chemical Formula 1A-II, and Chemical Formula 1A-III.

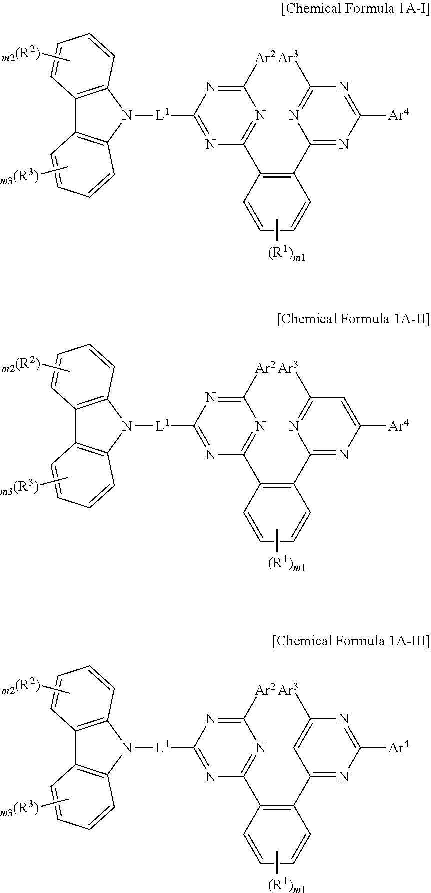

In Chemical Formula 1A-I, Chemical Formula 1A-II, and Chemical Formula 1A-III, Ar² to Ar⁴, L¹, R¹ to R³, and m1 to m3 are as defined in Chemical Formula 1A.

As a specific example, Chemical Formula 1B may be represented by Chemical Formula 1B-II or Chemical Formula 1B-III.

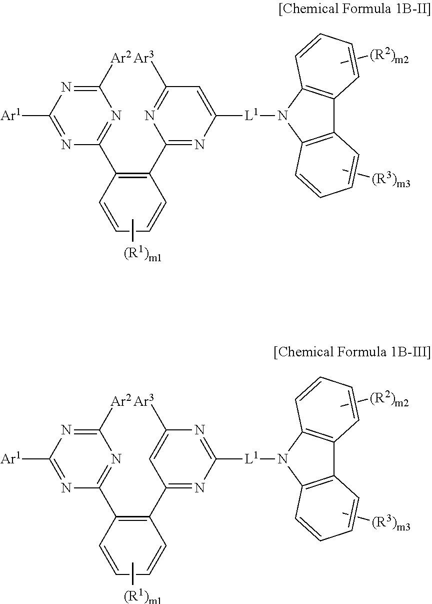

In Chemical Formula 1B-II and Chemical Formula 1B-III, Ar¹ to Ar³, L¹, R¹ to R³, and m1 to m3 are as defined in Chemical Formula 1B.

For example, in Chemical Formula 1, Ar¹ to Ar⁴ may each independently be a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted triphenylene group, a substituted or unsubstituted dibenzofuranyl group, a substituted or unsubstituted dibenzothiophenyl group, or a substituted or unsubstituted carbazolyl group, and
at least one of Ar¹ to Ar⁴ is the group represented by Chemical Formula A, and
Chemical Formula 1 satisfies at least one of the following conditions (ix) to (xi):
   (ix) at least one of the remaining substituents not substituted by Chemical Formula A among Ar¹ to Ar⁴ is a substituted or unsubstituted biphenyl group, a substituted or unsubstituted terphenyl group, a substituted or unsubstituted naphthyl group, a substituted or unsubstituted fluorenyl group, or a substituted or unsubstituted triphenylene group;
   (x) at least one of R² and R³ is a substituted or unsubstituted biphenyl group, a substituted or unsubstituted terphenyl group, a substituted or unsubstituted naphthyl group, a substituted or unsubstituted fluorenyl group, or a substituted or unsubstituted triphenylene group; and
   (xi) R¹ is a substituted or unsubstituted C6 to C12 aryl group.

For example, in Chemical Formula 1, R¹ to R³ may each independently be hydrogen, deuterium, a substituted or unsubstituted phenyl group, or a substituted or unsubstituted biphenyl group.

The compound for an organic optoelectronic device according to an embodiment may be represented by any one of Chemical Formula 1A-I, Chemical Formula 1A-II, and Chemical Formula 1B-II.

For example, the compound for an organic optoelectronic device represented by Chemical Formula 1 may be one selected from the compounds listed in Group 1, but is not limited thereto.

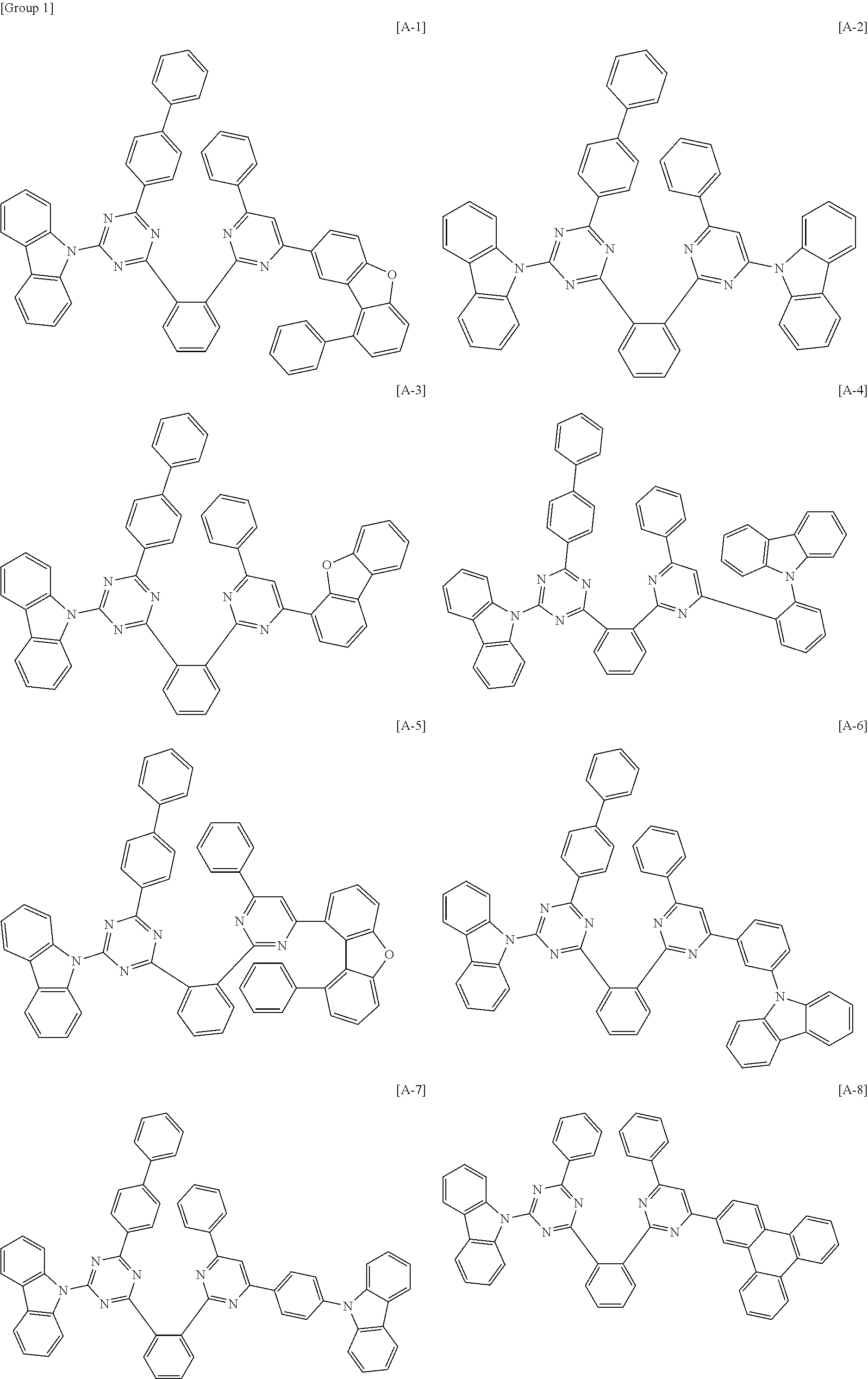

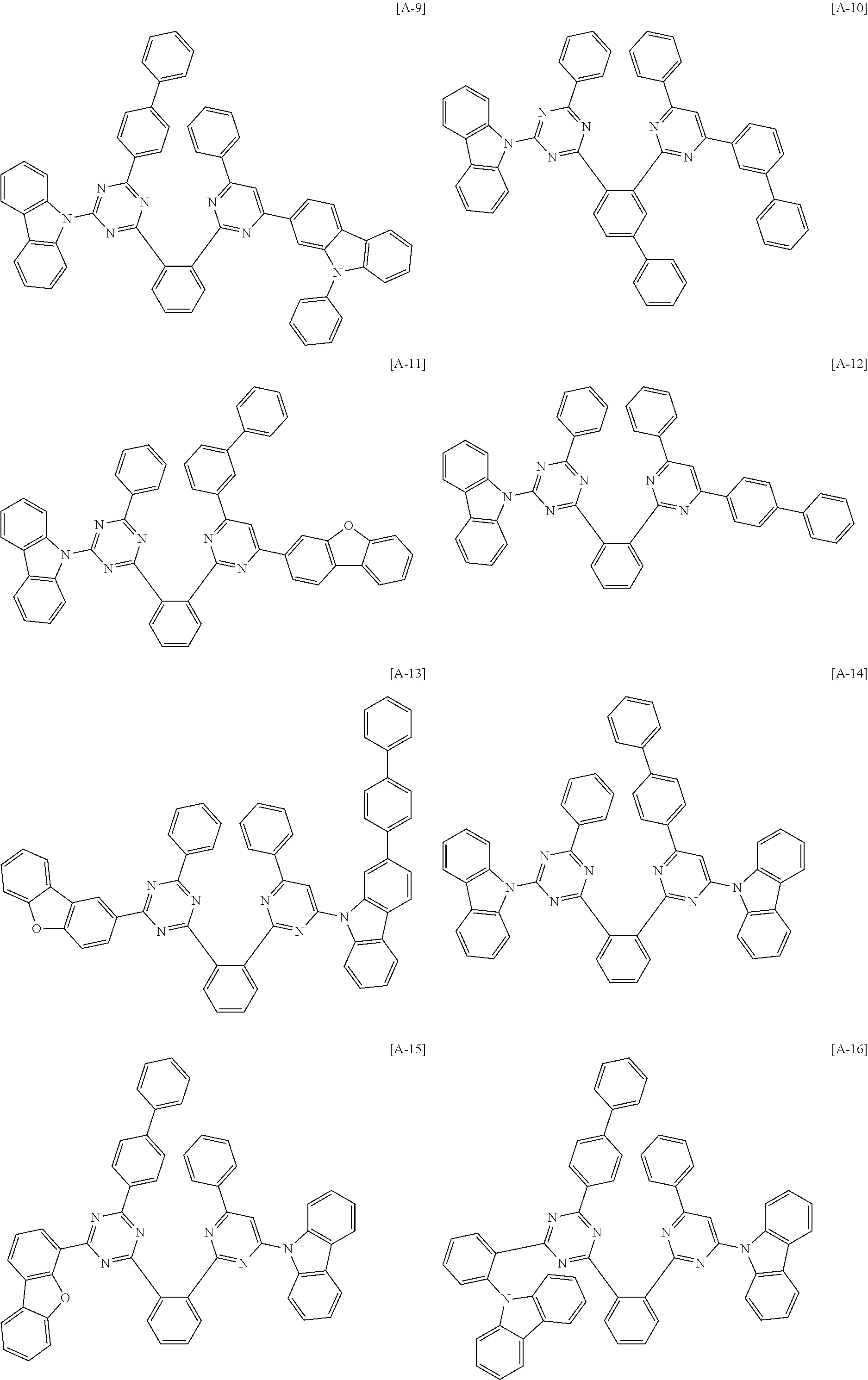

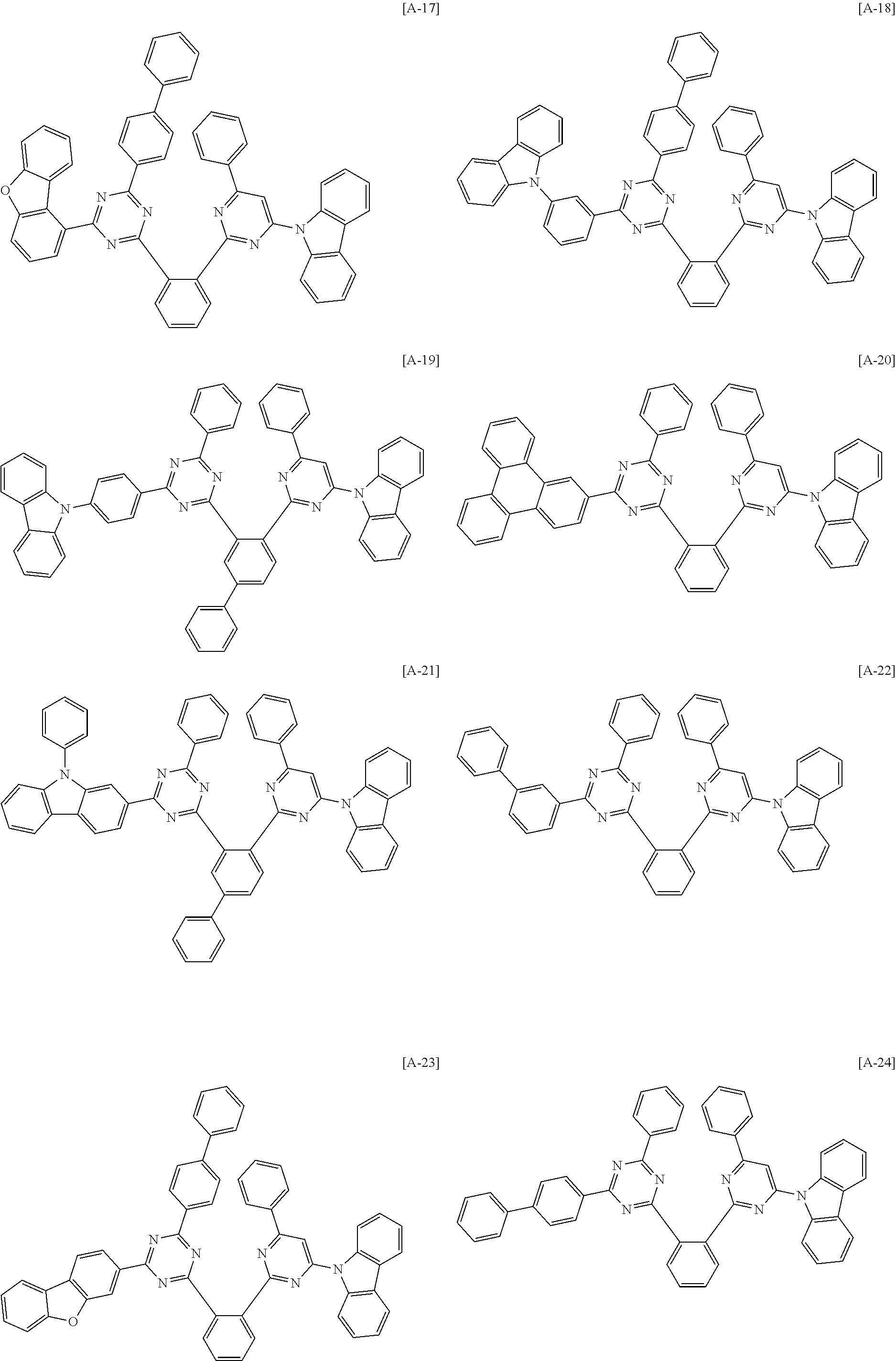

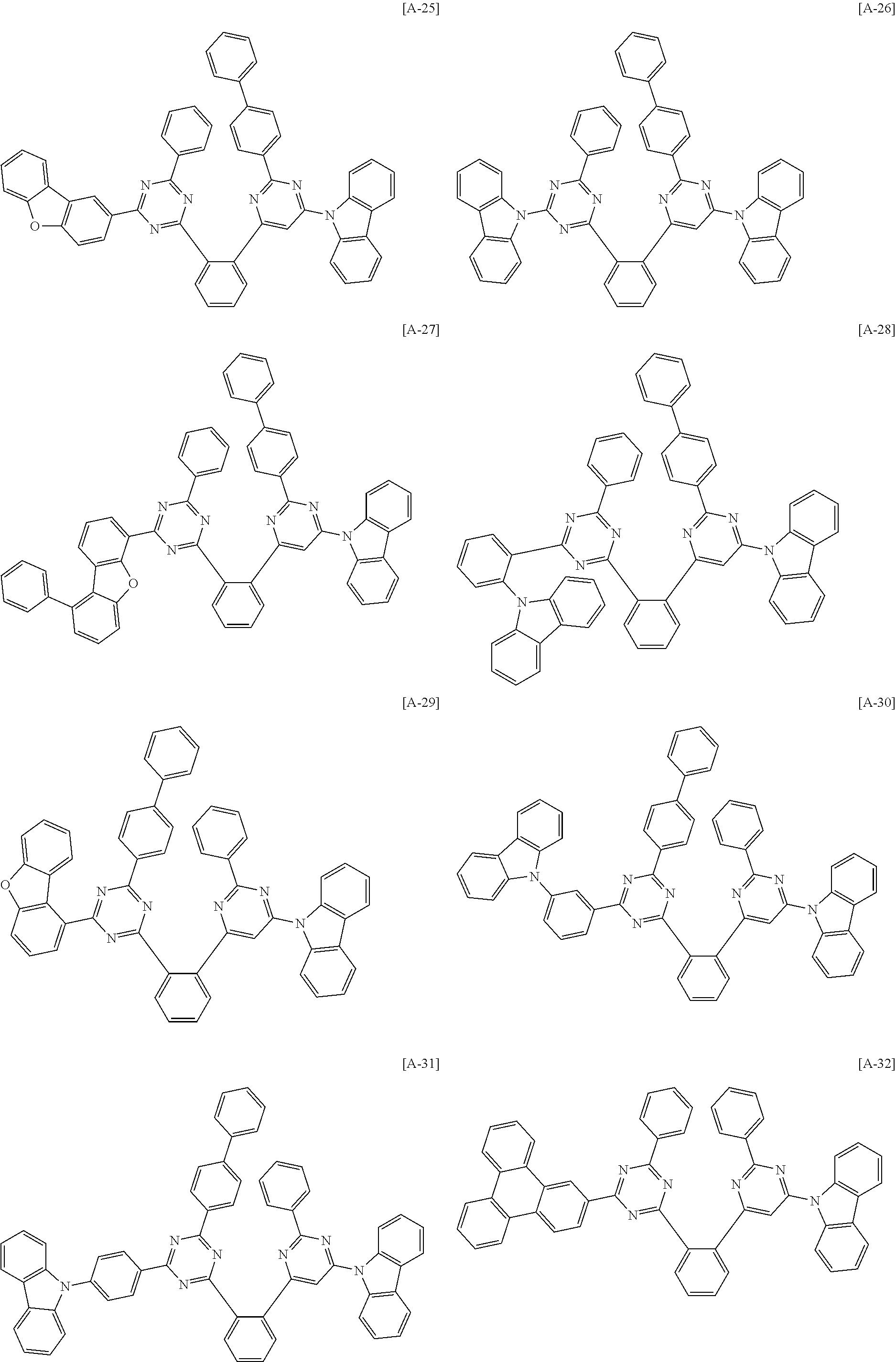

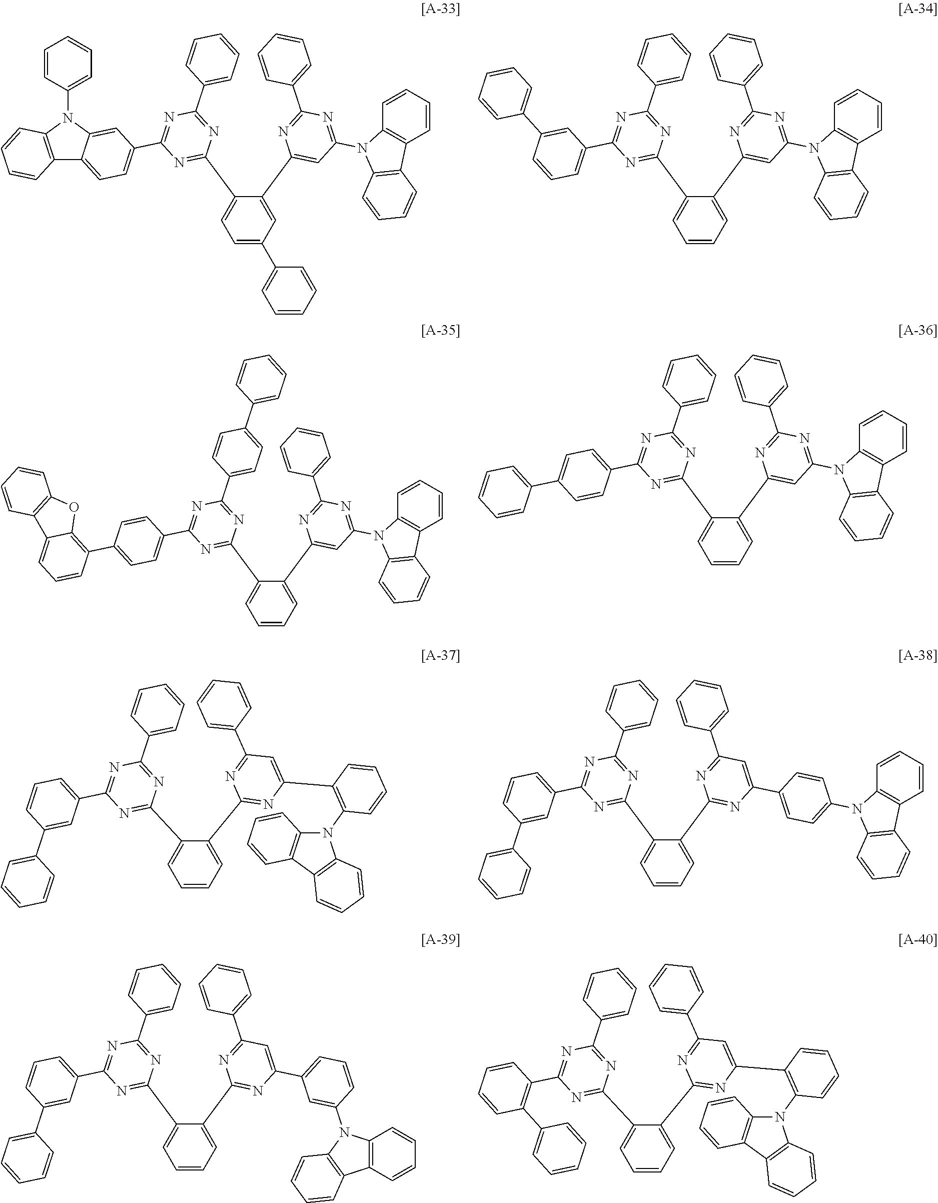

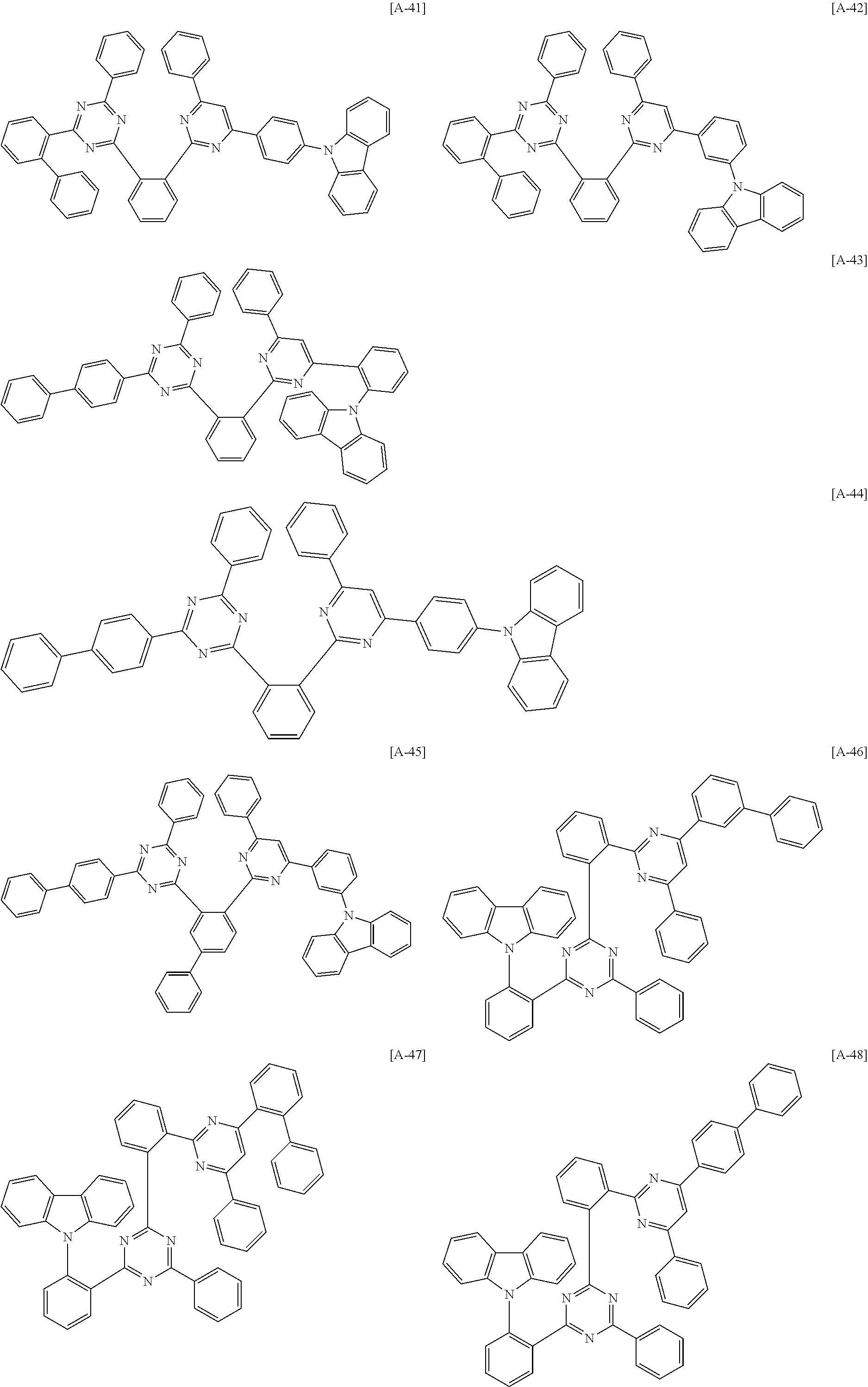

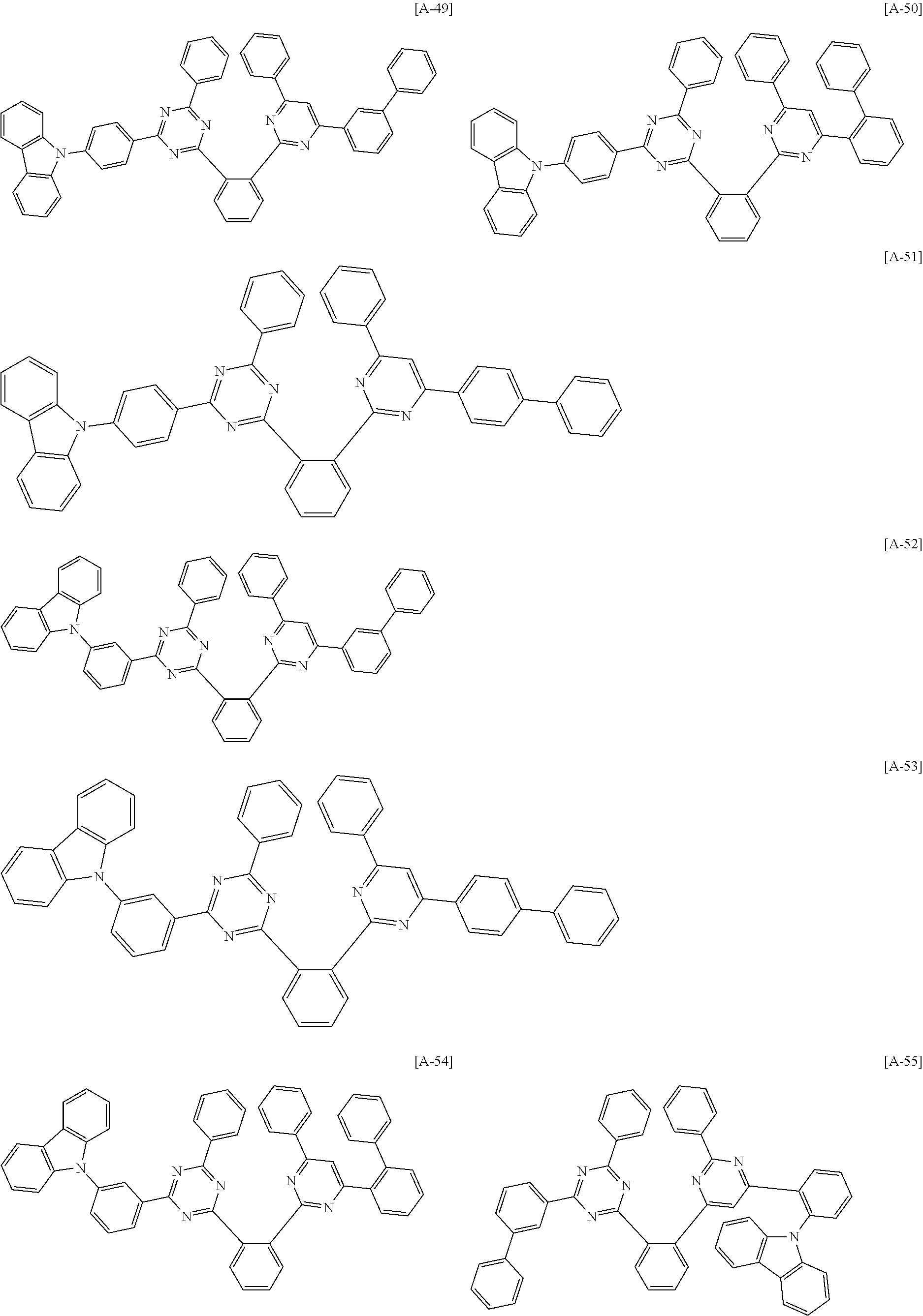

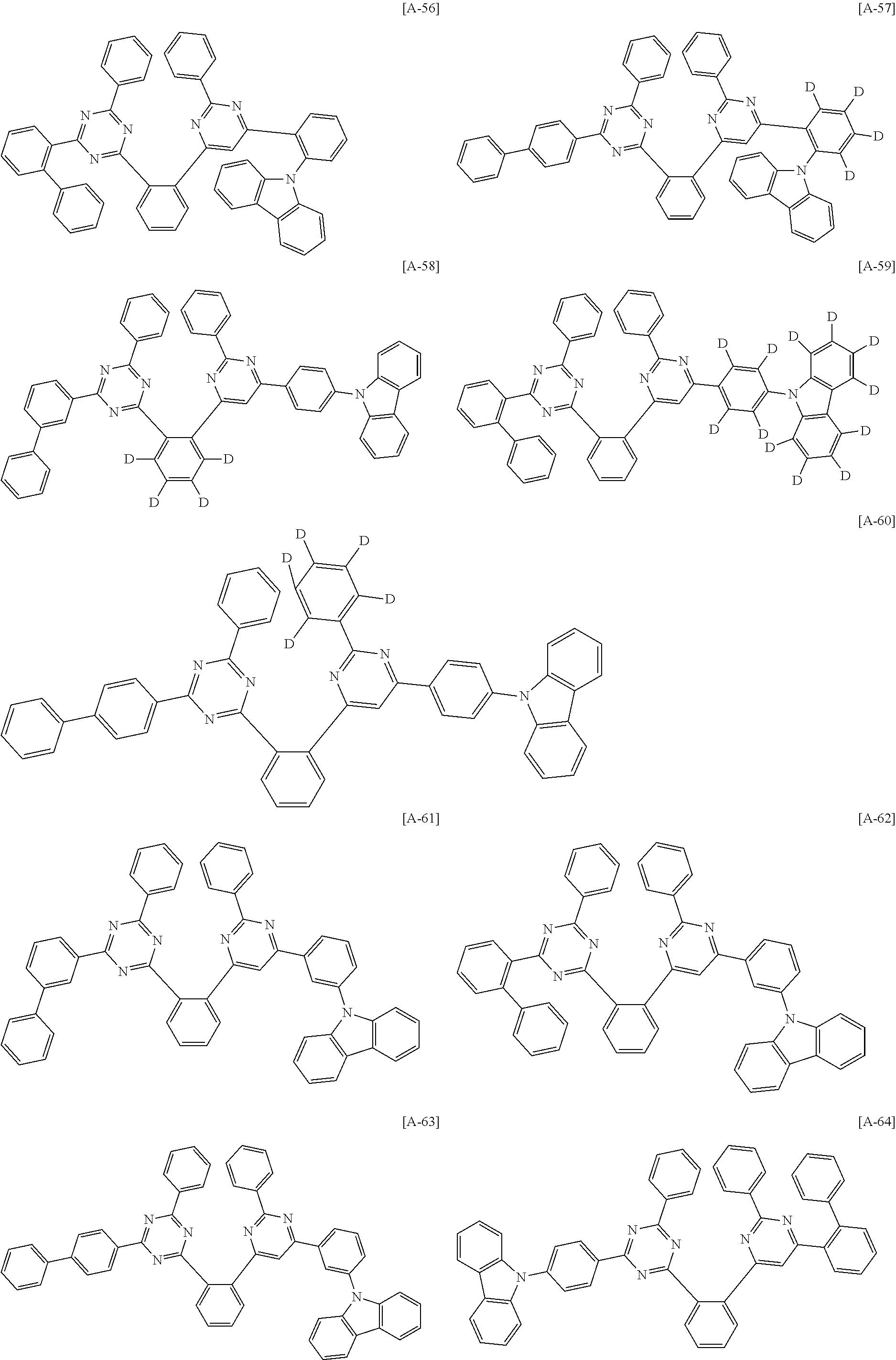

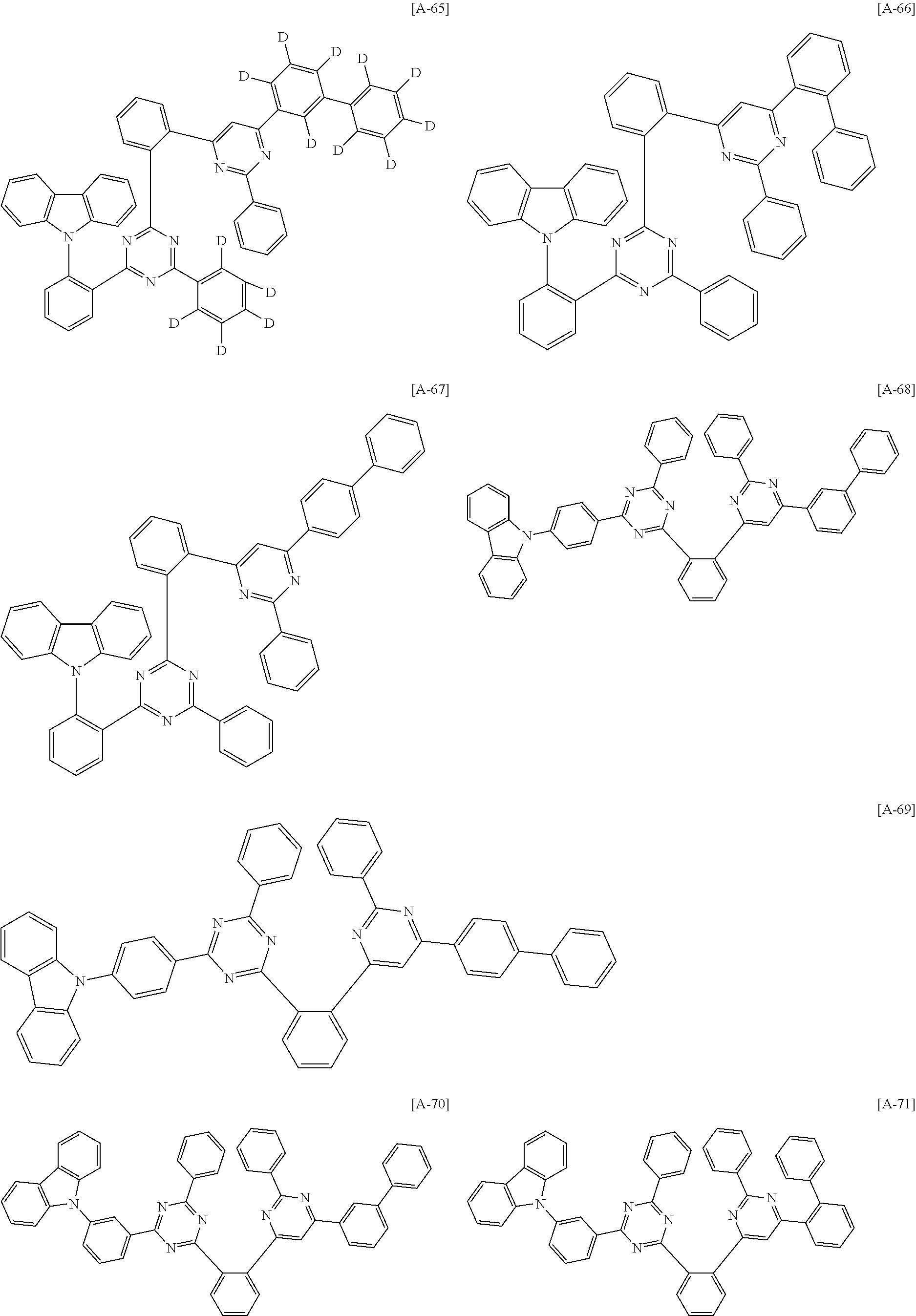

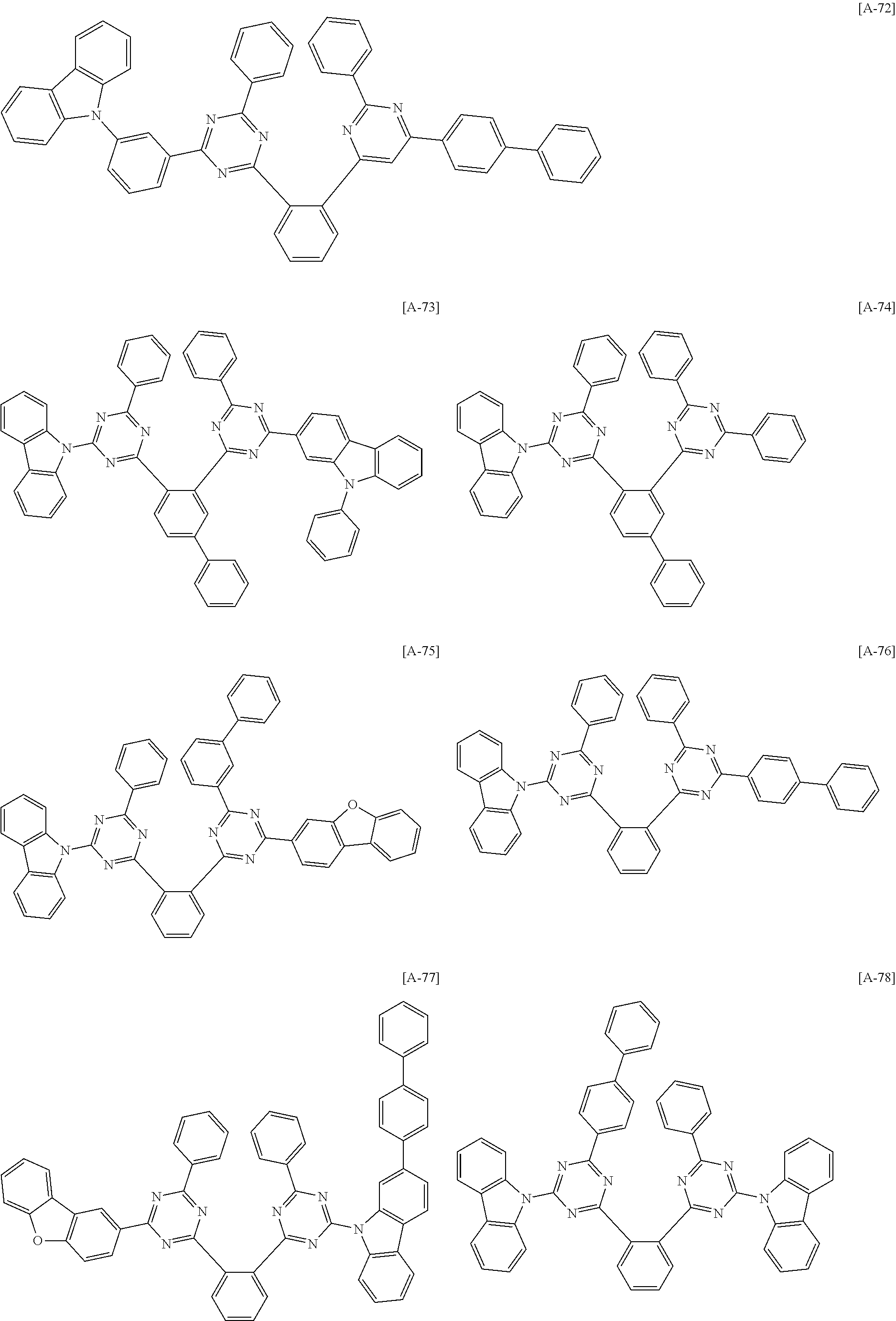

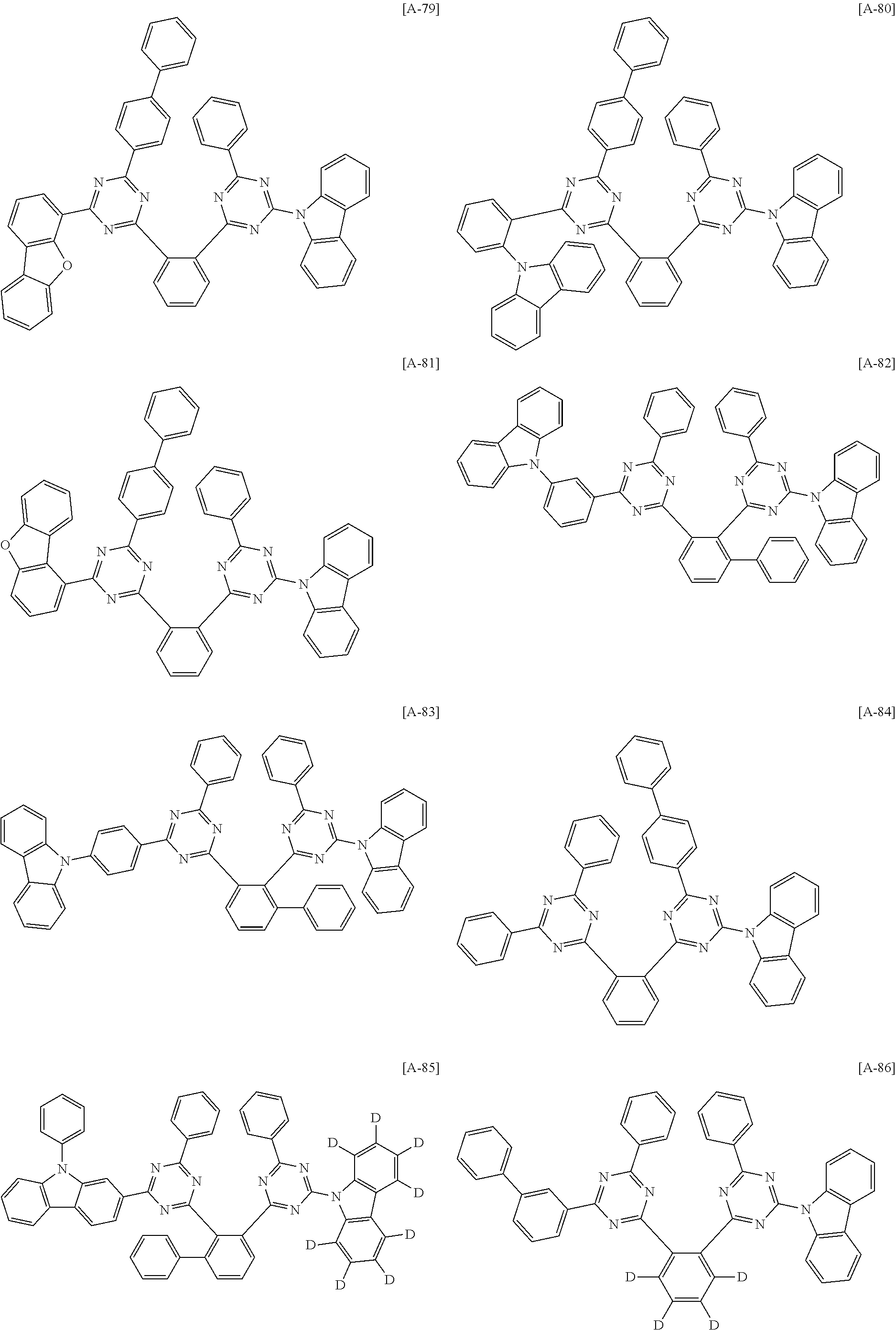

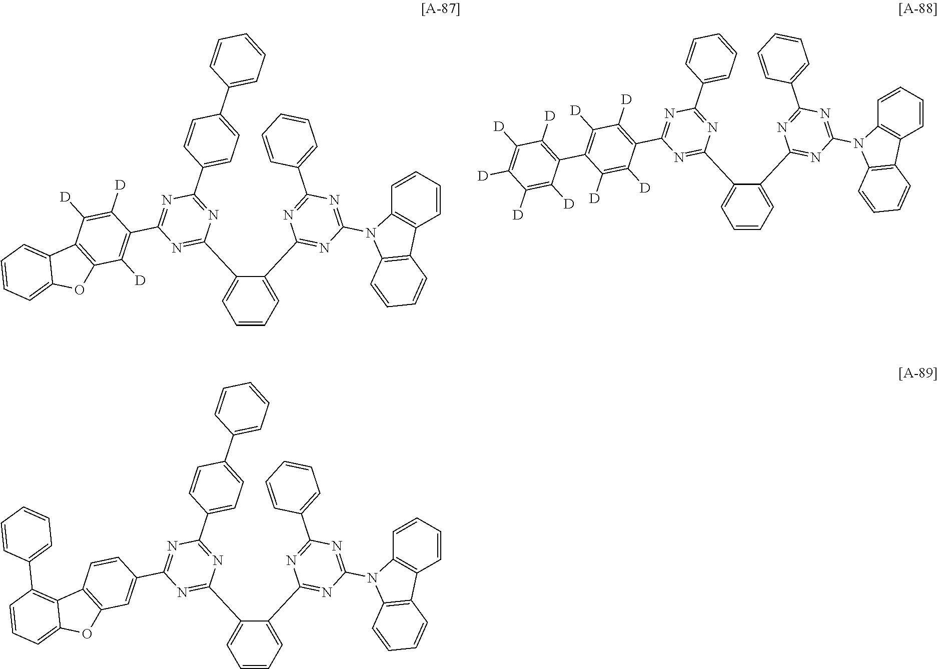 [SMILES: [2H]c1c(-c2nc(-c3ccc(-c4ccccc4)cc3)nc(-c3ccccc3-c3nc(-c4ccccc4)nc(-n4c5ccccc5c5ccccc54)n3)n2)c([2H])c2oc3ccccc3c2c1[2H] ; [2H]c1c([2H])c([2H])c(-c2c([2H])c([2H])c(-c3nc(-c4ccccc4)nc(-c4ccccc4-c4nc(-c5ccccc5)nc(-n5c6ccccc6c6ccccc65)n4)n3)c([2H])c2[2H])c([2H])c1[2H] ; c1ccc(-c2ccc(-c3nc(-c4ccc5c(c4)oc4cccc(-c6ccccc6)c45)nc(-c4ccccc4-c4nc(-c5ccccc5)nc(-n5c6ccccc6c6ccccc65)n4)n3)cc2)cc1]

A composition for an organic optoelectronic device according to another embodiment includes a first compound and a second compound, wherein the first compound may be the aforementioned compound for an organic optoelectronic device, and the second compound may be represented by Chemical Formula 2; or a combination of Chemical Formula 3 and Chemical Formula 4.

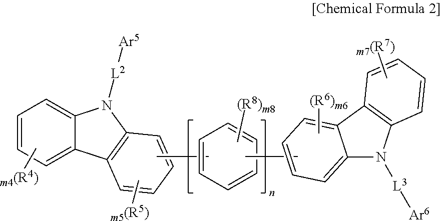

In Chemical Formula 2,
R⁴ to R⁸ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁵ and Ar⁶ are each independently a substituted or unsubstituted C6 to C20 aryl group or a substituted or unsubstituted C2 to C30 heterocyclic group,
L² and L³ are each independently a single bond or a substituted or unsubstituted C6 to C20 arylene group,
m4, m7, and m8 are each independently one of integers of 1 to 4,
m5 and m6 are each independently one of integers of 1 to 3, and
n is one of integers of 0 to 2;

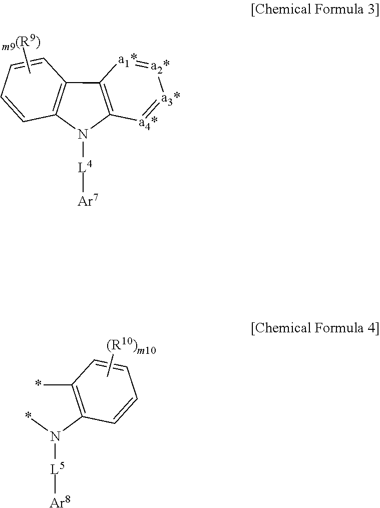

In Chemical Formula 3 and Chemical Formula 4,
a₁* to a₄* in Chemical Formula 3 are each independently linking carbon (C) or C-L^{a}-R^{a}
among a₁* to a₄* in Chemical Formula 3, two adjacent ones are each linked to *s in Chemical Formula 4,
L^{a}, L⁴ and L⁵ are each independently a single bond or a substituted or unsubstituted C6 to C20 arylene group,
R^{a}, R⁹ and R¹⁰ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁷ and Ar⁸ are each independently a substituted or unsubstituted C6 to C20 aryl group or a substituted or unsubstituted C2 to C30 heterocyclic group, and
m9 and m10 are each independently one of integers of 1 to 4.

The second compound can be used in the light emitting layer together with the first compound to improve luminous efficiency and life-span characteristics by increasing charge mobility and stability.

In Chemical Formula 2, when m2 is 2 or more, each R⁴ may be the same or different from each other.

In Chemical Formula 2, when m5 is 2 or more, each R⁵ may be the same or different from each other.

In Chemical Formula 2, when m6 is 2 or more, each R⁶ may be the same or different from each other.

In Chemical Formula 2, when m7 is 2 or more, each R⁷ may be the same or different from each other.

In Chemical Formula 2, when m8 is 2 or more, each R⁸ may be the same or different from each other.

As an example, in Chemical Formula 2, Ar⁵ and Ar⁶ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group,
in Chemical Formula 2, L² and L³ may each independently be a single bond, a substituted or unsubstituted phenylene group, or a substituted or unsubstituted biphenylene group,
in Chemical Formula 2, R⁴ to R⁷ may each independently be hydrogen, deuterium, or a substituted or unsubstituted C6 to C12 aryl group, and
n may be 0 or 1.

As an example, in Chemical Formula 2, “substituted” refers to replacement of at least one hydrogen by deuterium, a C1 to C4 alkyl group, a C6 to C18 aryl group, or a C2 to C30 heteroaryl group.

In a specific embodiment of the present invention, Chemical Formula 2 may be represented by any one of Chemical Formula 2-1 to Chemical Formula 2-15.

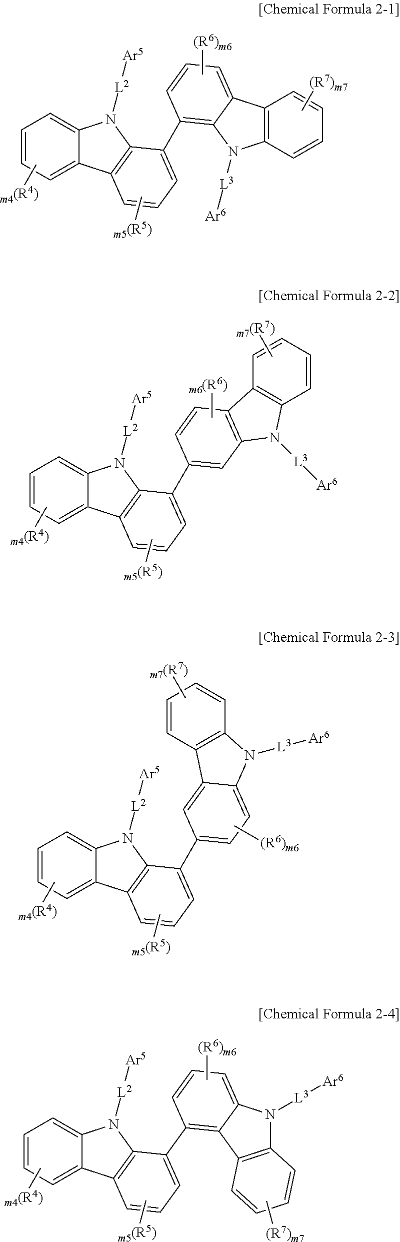

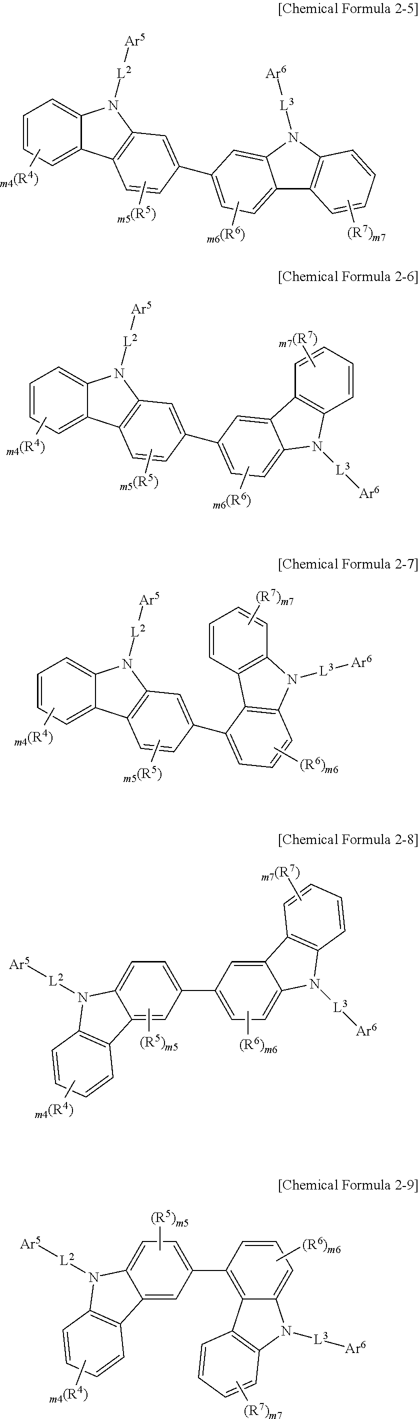

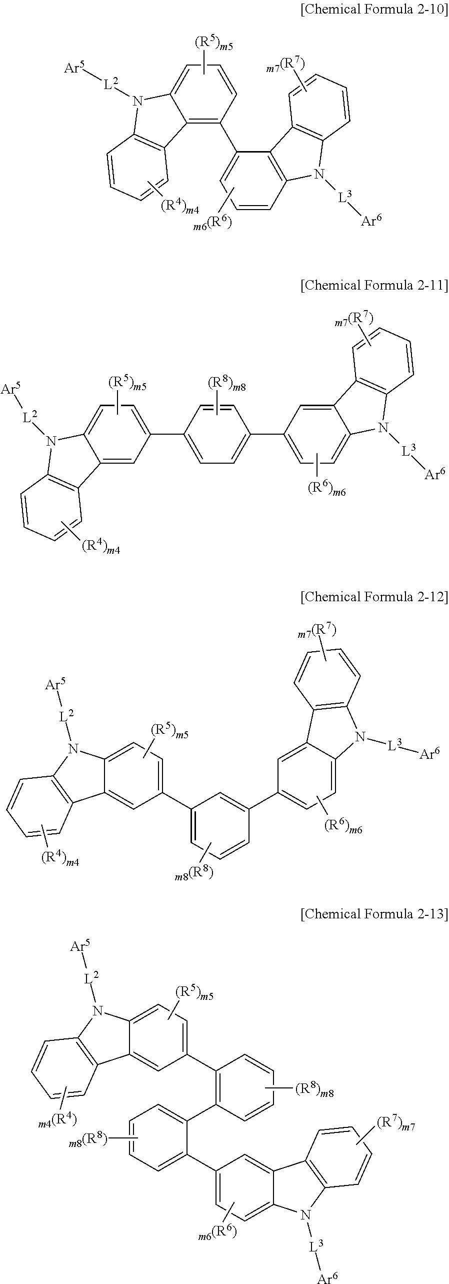

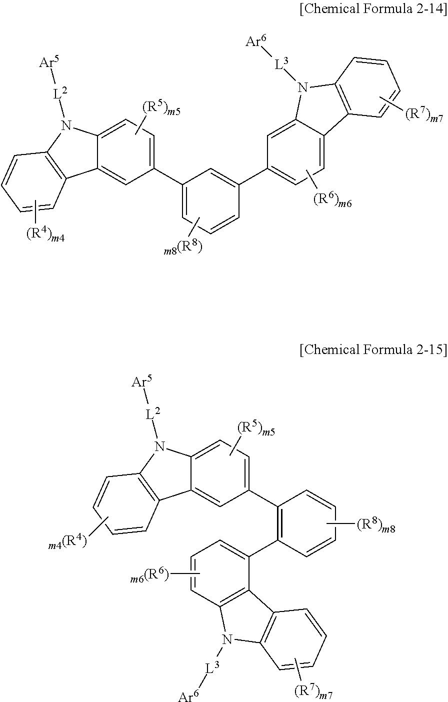

In Chemical Formula 2-1 to Chemical Formula 2-15, R⁴ to R⁸ may each independently be hydrogen, deuterium, or a substituted or unsubstituted C6 to C12 aryl group, and *-L²-Ar⁵ and *-L³-Ar⁶ may each independently be one of the substituents listed in Group I.

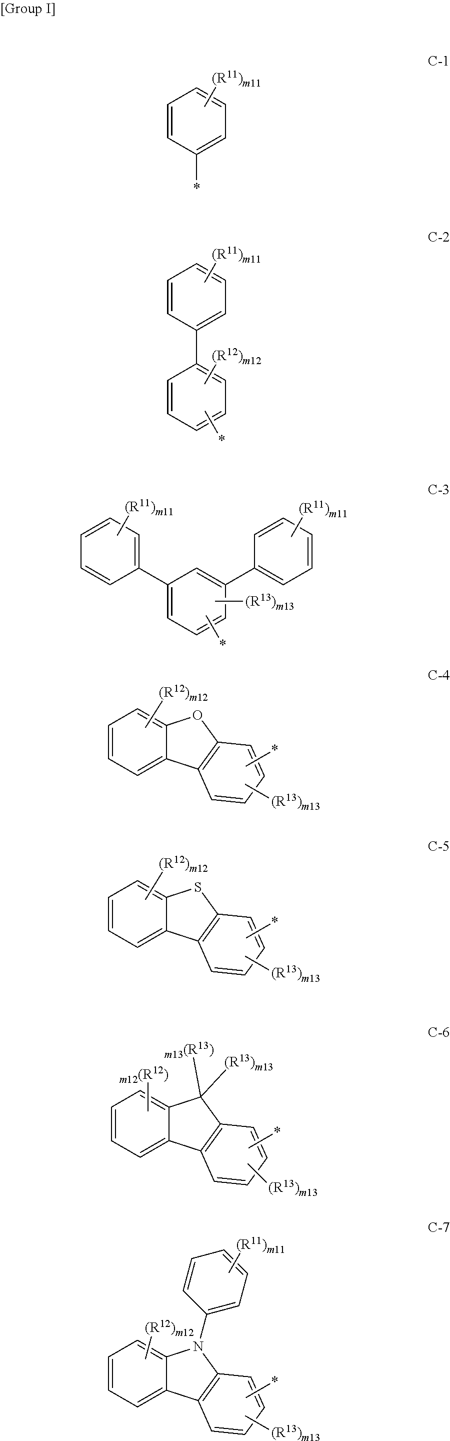

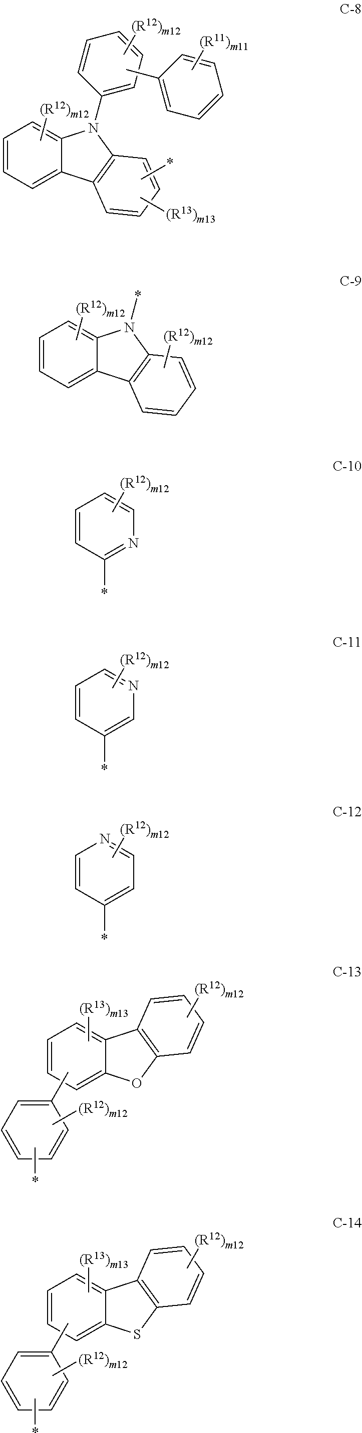

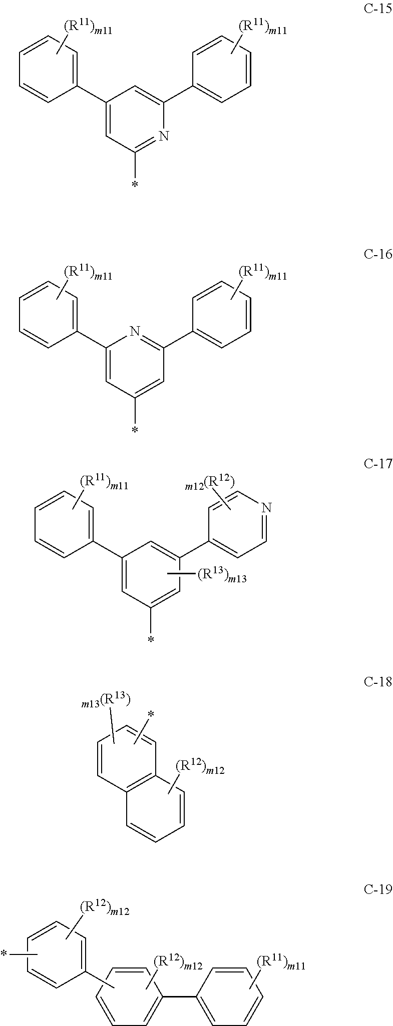

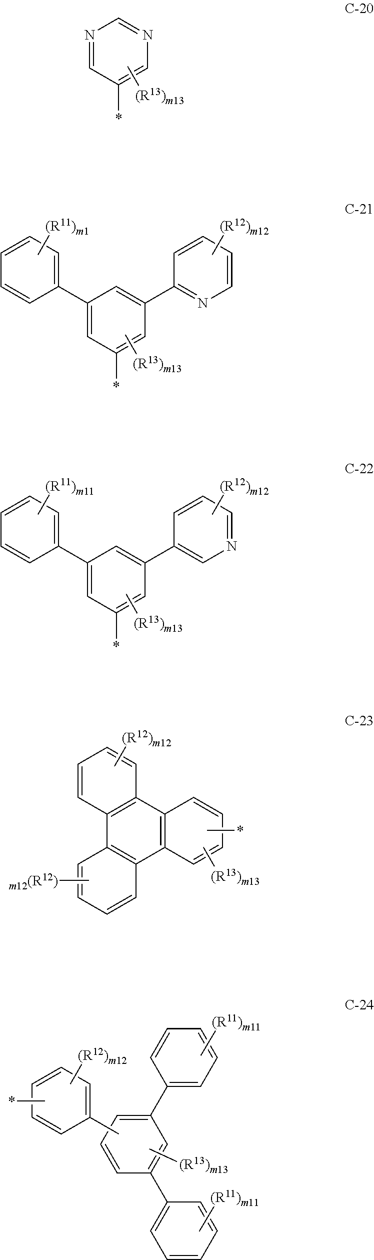

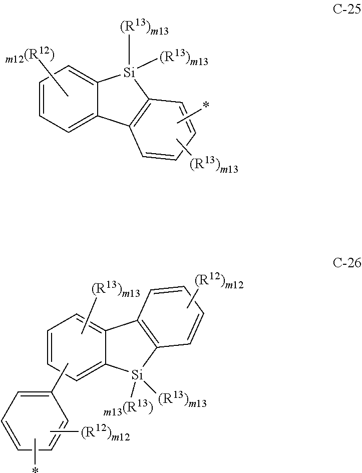

In Group I,
R¹¹ to R¹³ are each independently hydrogen, deuterium, a substituted or unsubstituted C1 to C4 alkyl group, a substituted or unsubstituted C6 to C18 aryl group, or a substituted or unsubstituted C2 to C30 heteroaryl group,
m11 is one of integers of 1 to 5,
m12 is one of integers of 1 to 4,
m13 is one of integers of 1 to 3, and
* is a linking point.

In Group I, when m11 is 2 or more, each R¹¹ may be the same or different from each other.

In Group I, when m12 is 2 or more, each R¹² may be the same or different from each other.

In Group I, when m13 is 2 or more, each R¹³ may be the same or different from each other.

In an embodiment, Chemical Formula 2 may be represented by Chemical Formula 2-8.

In addition, in Chemical Formula 2-8, *-L²-Ar⁵ and *-L³-Ar⁶ may each independently selected from Group I.

In Chemical Formula 3 and Chemical Formula 4, when m9 is 2 or more, each R⁹ may be the same or different from each other.

In Chemical Formula 3 and Chemical Formula 4, when m10 is 2 or more, each R¹⁰ may be the same or different from each other.

For example, the second compound represented by combination of Chemical Formula 3 and Chemical Formula 4 may be represented by any one of Chemical Formula 3A, Chemical Formula 3B, Chemical Formula 3C, Chemical Formula 3D, and Chemical Formula 3E.

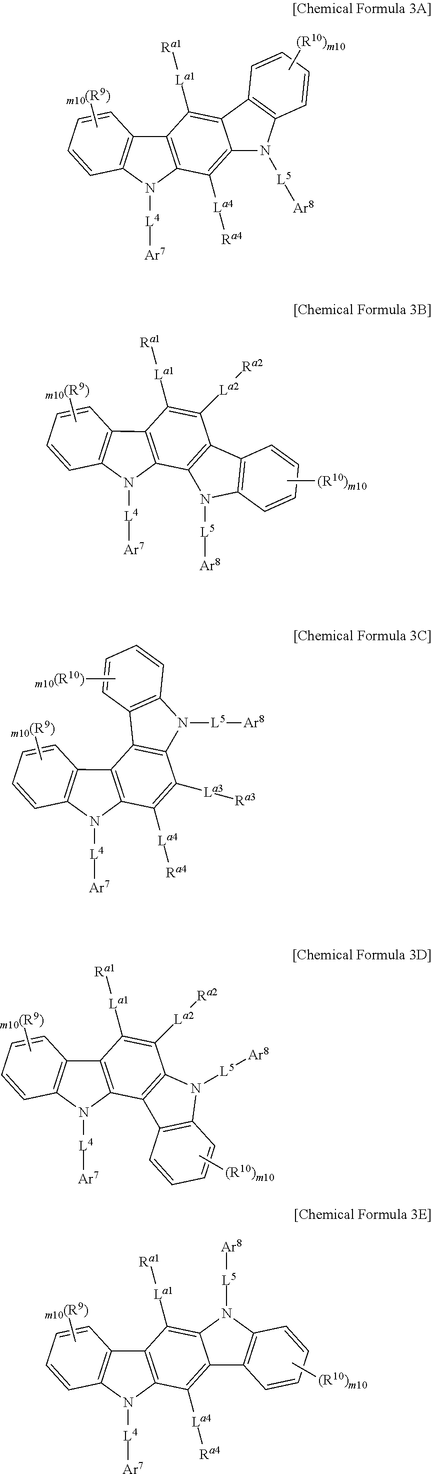

In Chemical Formula 3A to Chemical Formula 3E, L⁴, L⁵, R⁹, and R¹⁰ are the same as described above,
L^{a1} to L^{a4} are the same as the definitions of L⁴ and L⁵, and
R^{a1} to R^{a4} are the same as the definitions of R⁹ and R¹⁰.

For example, in Chemical Formulas 3 and 4, Ar⁷ and Ar⁸ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, and
R^{a1} to R^{a4}, R⁹, and R¹⁰ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In a specific embodiment of the present invention, in Chemical Formulas 3 and 4, *-L⁴-Ar⁷ and *-L⁵-Ar⁸ may each independently be selected from substituents listed in Group I.

In an embodiment, R^{a1} to R^{a4}, R⁹ and R¹⁰ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

For example, R^{a1} to R^{a4}, R⁹ and R¹⁰ may each independently be hydrogen, deuterium, a cyano group, or a substituted or unsubstituted phenyl group, and
in an embodiment, R^{a1} to R^{a4}, R⁹, and R¹⁰ may each independently be hydrogen, deuterium, or a substituted or unsubstituted phenyl group.

In a specific embodiment of the present invention, the second compound may be represented by Chemical Formula 2-8, and in Chemical Formula 2-8, Ar⁵ and Ar⁶ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L² and L³ may each independently be a single bond, or a substituted or unsubstituted C6 to C20 arylene group, and R⁴ to R⁷ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In a specific embodiment of the present invention, the second compound may be represented by Chemical Formula 3C, and in Chemical Formula 3C, L^{a3} and L^{a4} may be a single bond, L⁴ and L⁵ may each independently be a single bond or a substituted or unsubstituted C6 to C12 arylene group, R⁹, R¹⁰, R^{a3}, and R^{a4} may each independently be hydrogen, deuterium, or a phenyl group, and Ar⁷ and Ar⁸ may each independently be a substituted or unsubstituted phenyl group, a substituted or unsubstituted biphenyl group, or a substituted or unsubstituted terphenyl group.

For example, the second organic optoelectronic device compound may be one selected from the compounds listed in Group 2, but is not limited thereto.

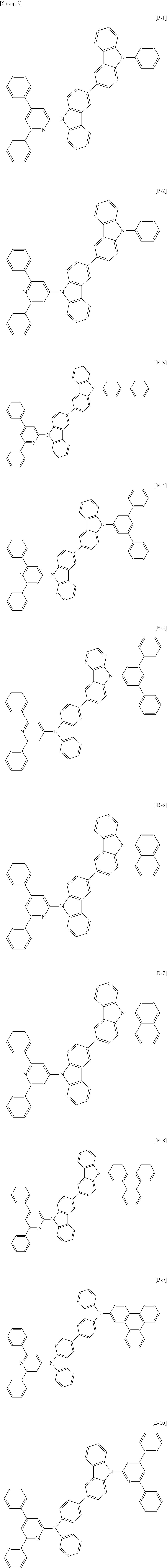

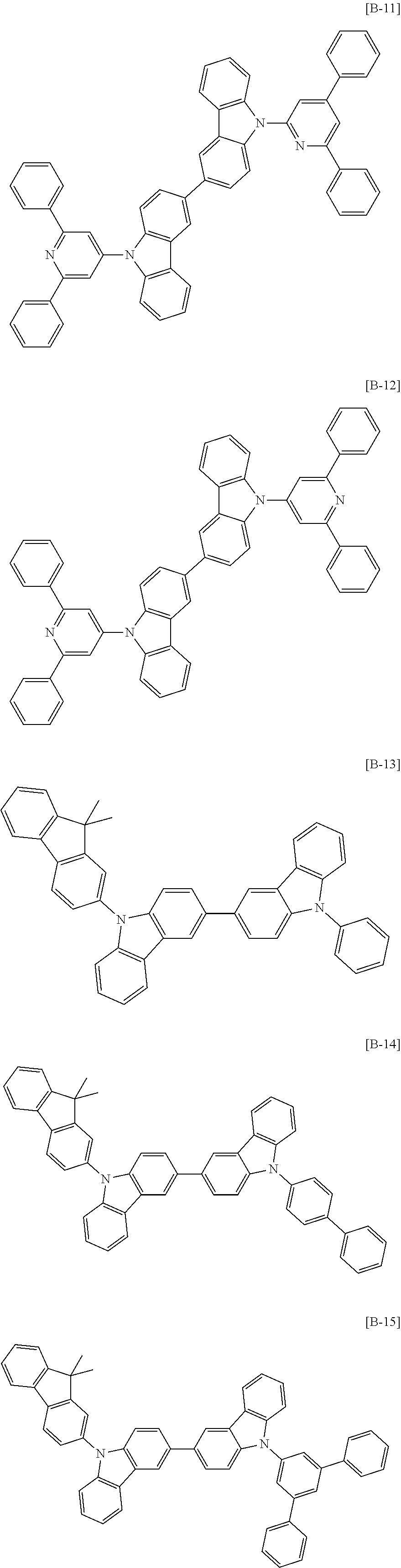 [SMILES: c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1 ; CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21]

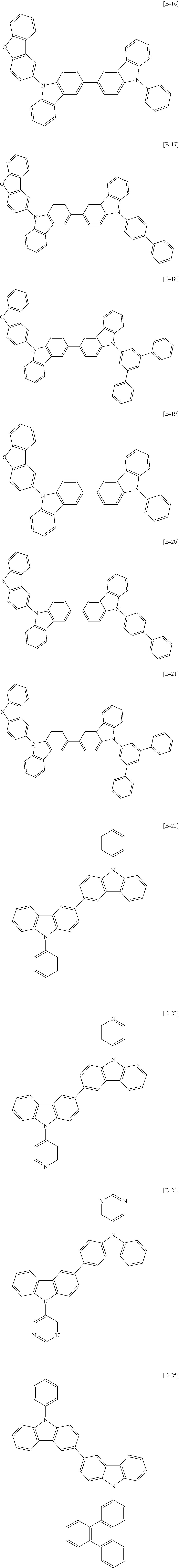

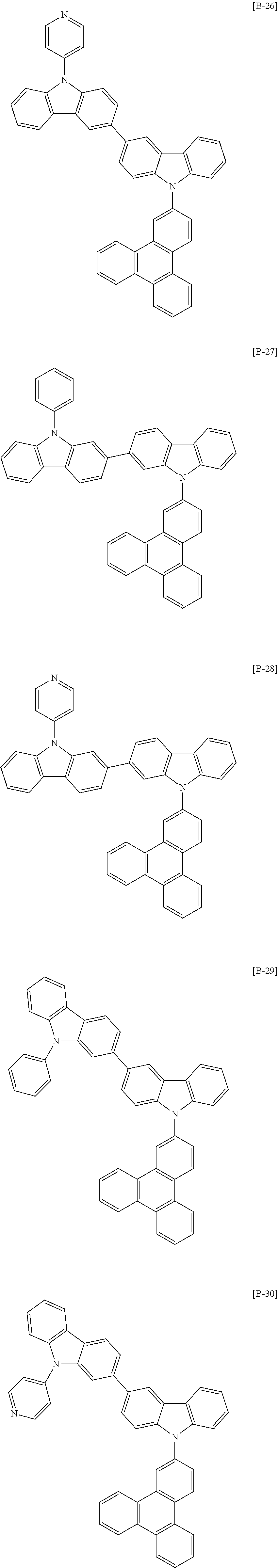 [SMILES: c1ccc2c(c1)c1ccccc1c1cc(-n3c4ccccc4c4cc(-c5ccc6c(c5)c5ccccc5n6-c5ccncc5)ccc43)ccc21 ; c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21 ; c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21]

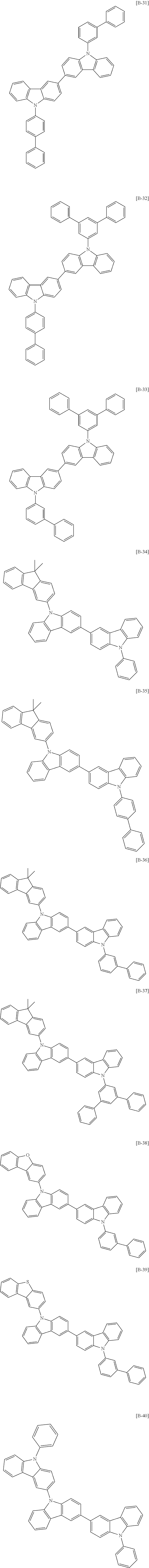

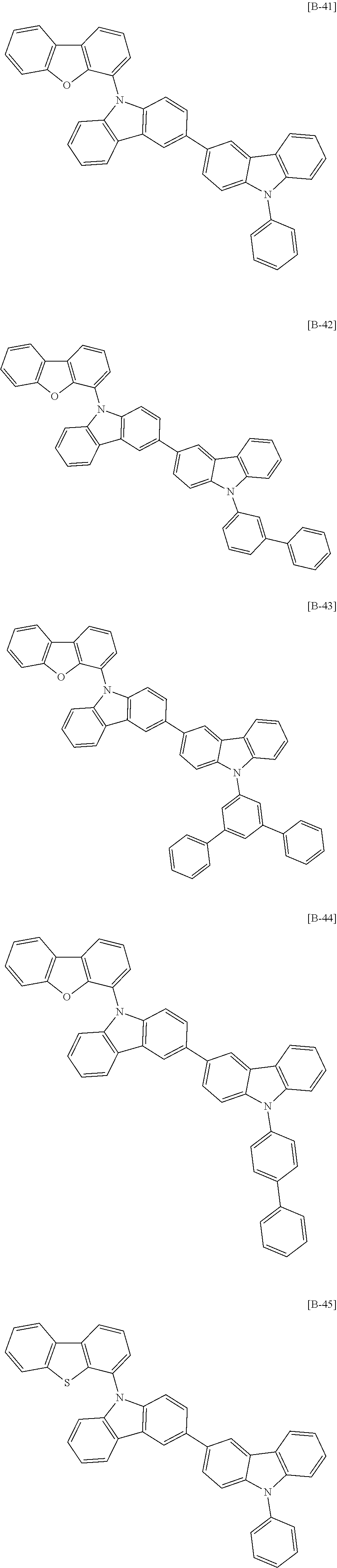 [SMILES: c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5cccc6c5oc5ccccc56)ccc43)cc2)cc1 ; c1ccc(-n2c3ccccc3c3cc(-c4ccc5c(c4)c4ccccc4n5-c4cccc5c4sc4ccccc45)ccc32)cc1]

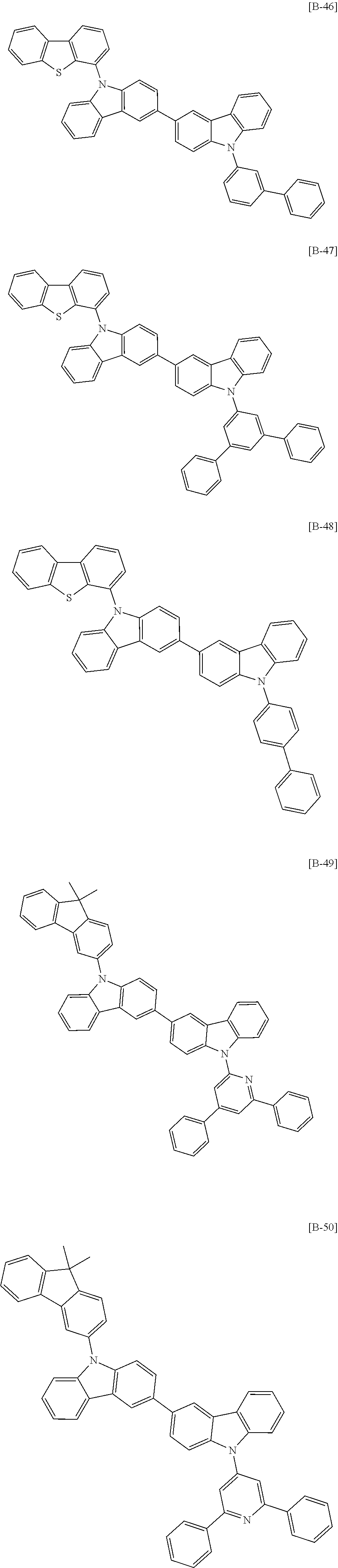 [SMILES: c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5ccc6c(c5)c5ccccc5n6-c5cccc6c5sc5ccccc56)ccc43)cc2)cc1 ; CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21]

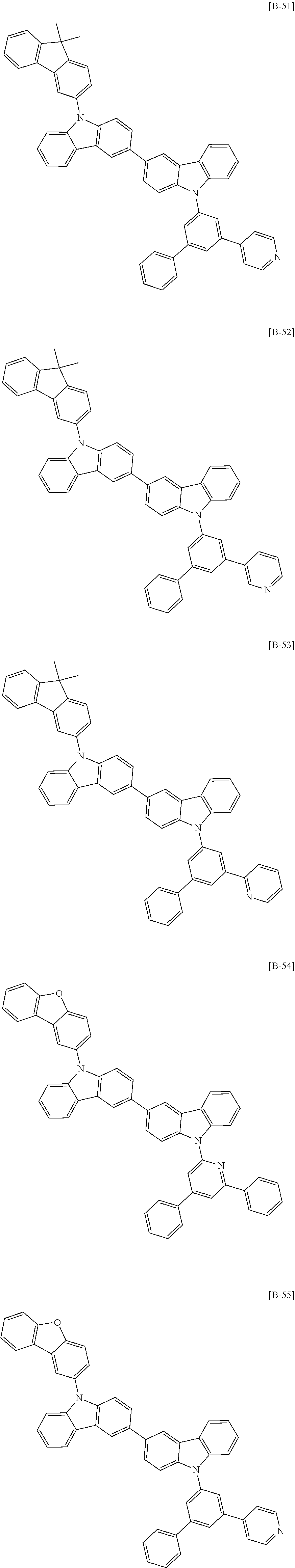

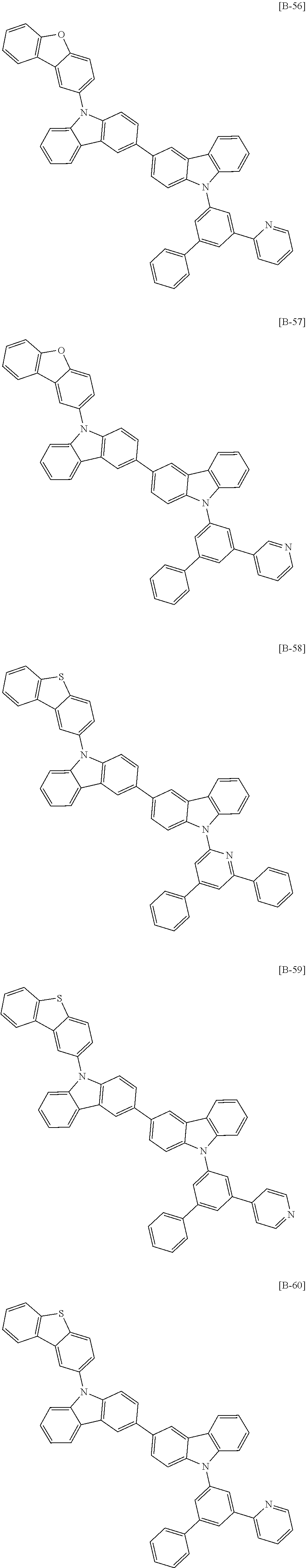 [SMILES: c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1]

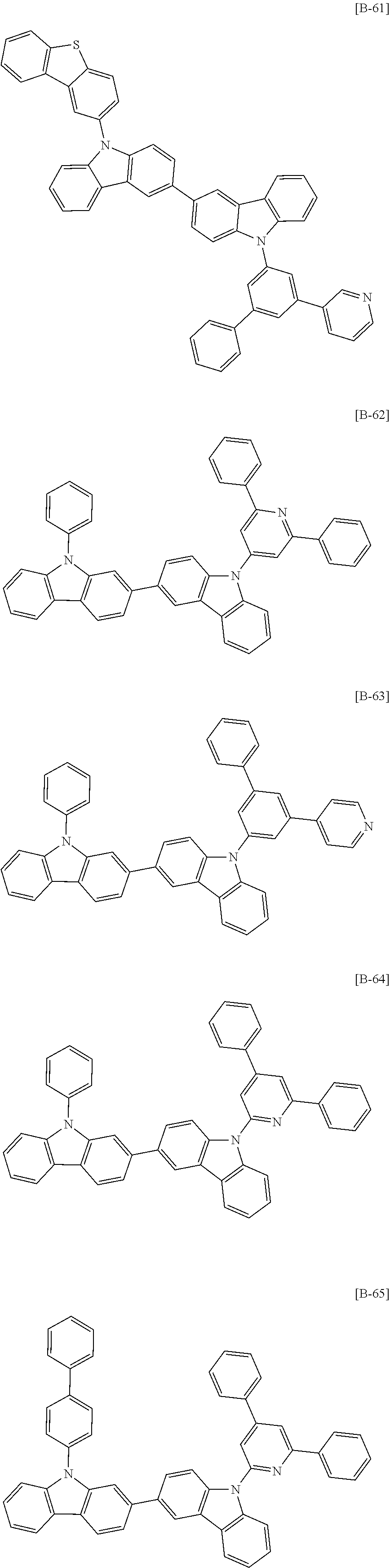 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1 ; c1ccc(-c2cc(-c3ccccc3)nc(-n3c4ccccc4c4cc(-c5ccc6c7ccccc7n(-c7ccccc7)c6c5)ccc43)c2)cc1 ; c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1]

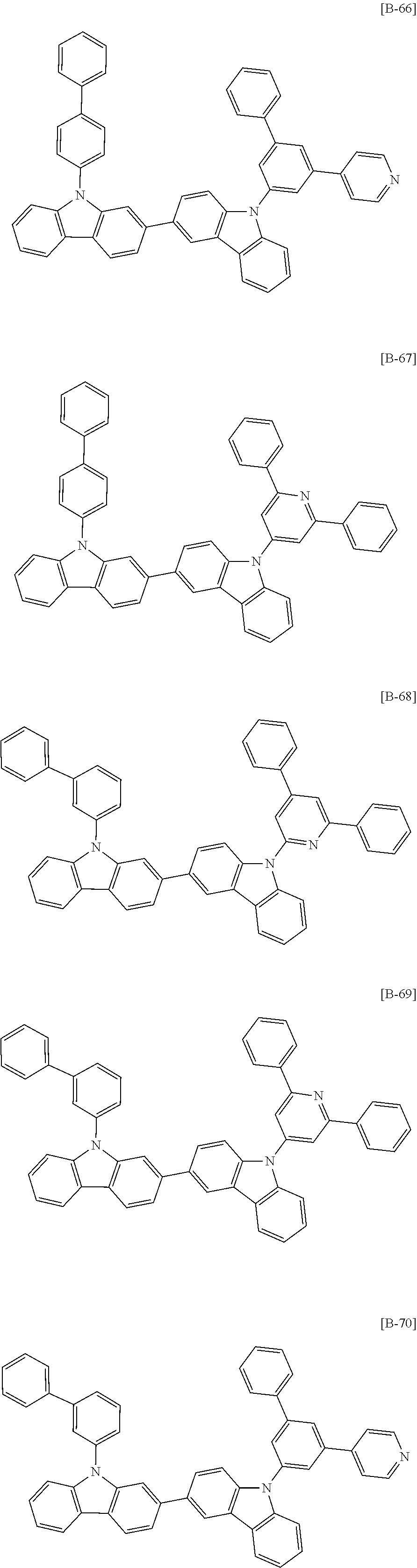 [SMILES: c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1 ; c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1 ; c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1]

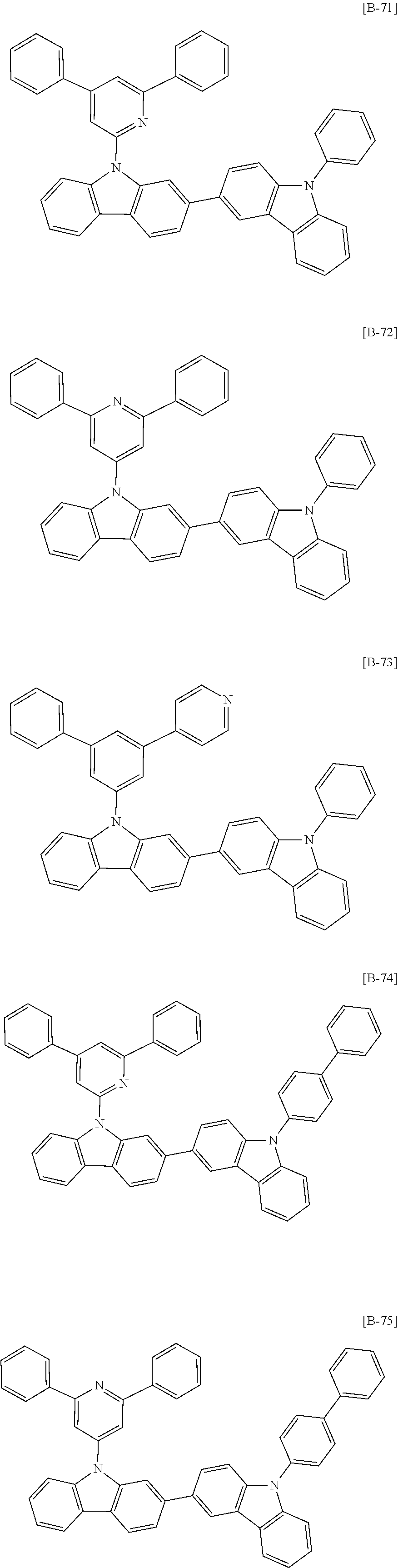 [SMILES: c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cc(-n3c4ccccc4c4ccc(-c5ccc6c(c5)c5ccccc5n6-c5ccccc5)cc43)cc(-c3ccccc3)n2)cc1 ; c1ccc(-c2cc(-c3ccncc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1 ; c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1]

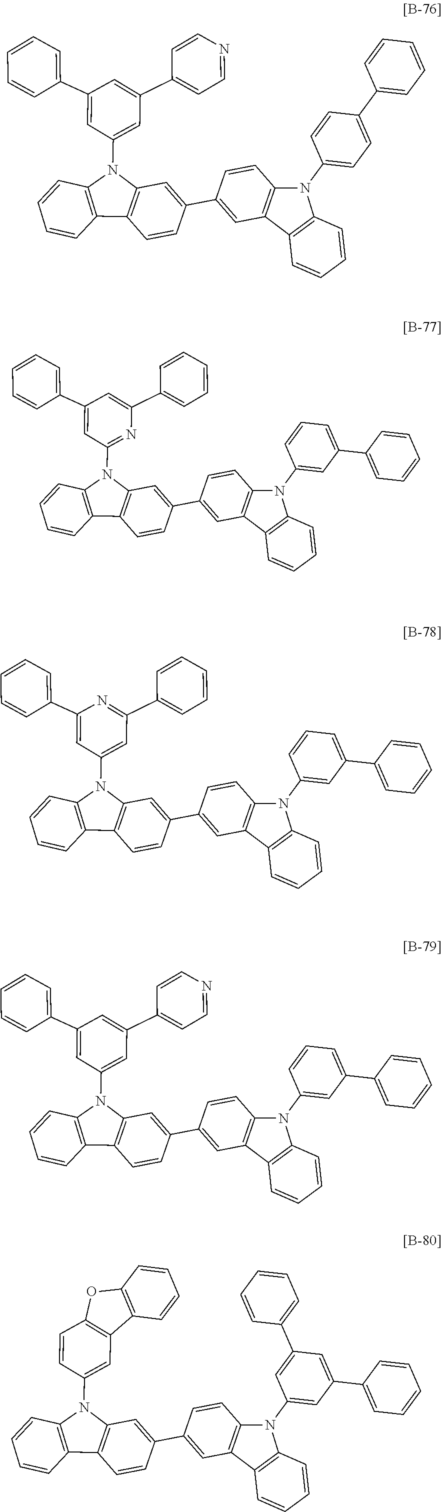 [SMILES: c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1 ; c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1]

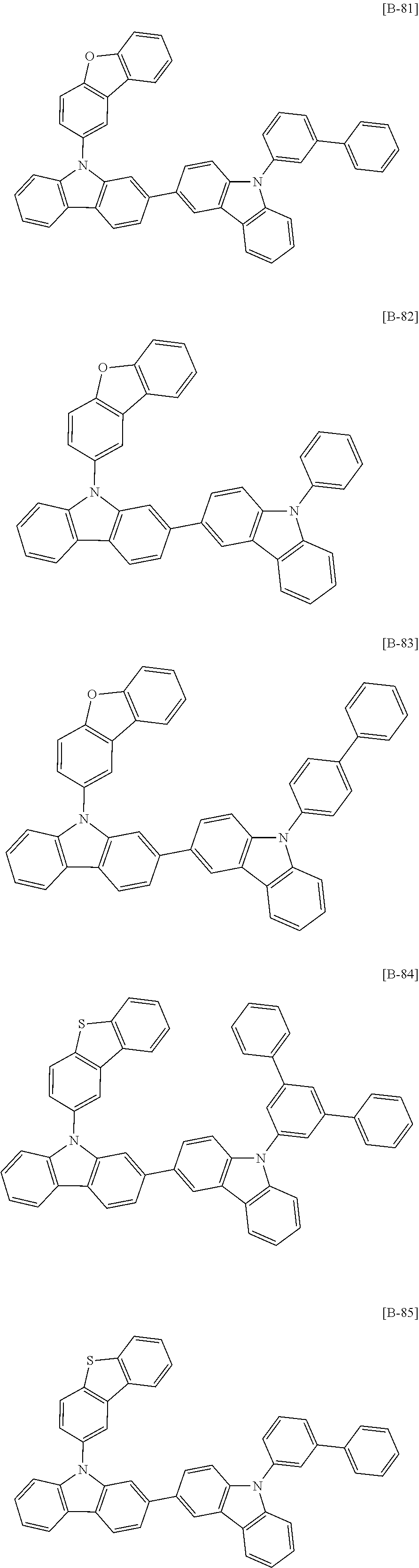 [SMILES: c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1 ; c1ccc(-c2ccc(-n3c4ccccc4c4cc(-c5ccc6c7ccccc7n(-c7ccc8oc9ccccc9c8c7)c6c5)ccc43)cc2)cc1 ; c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1]

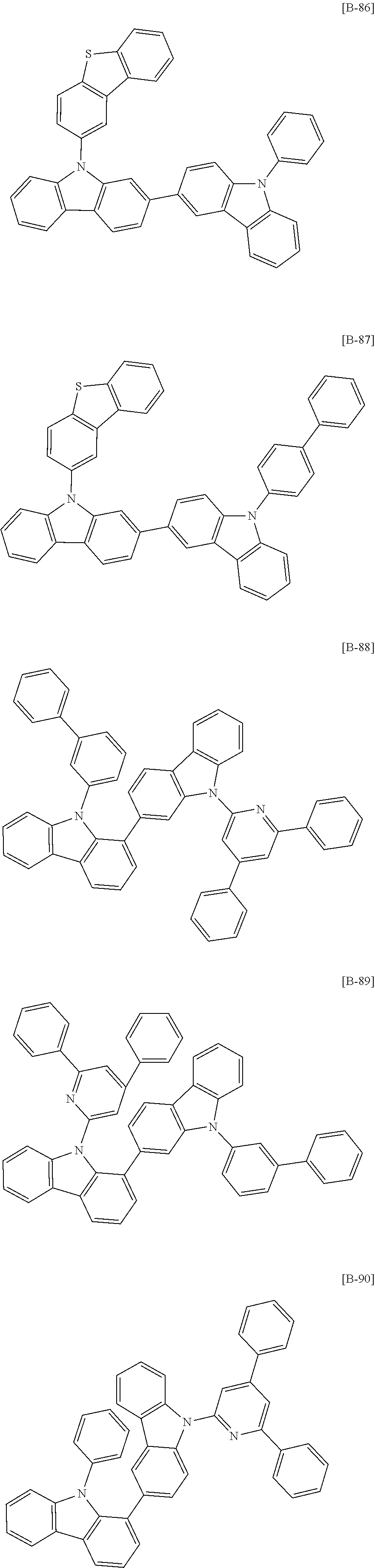 [SMILES: c1ccc(-n2c3ccccc3c3cc(-c4ccc5c6ccccc6n(-c6ccc7sc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1 ; c1ccc(-c2cccc(-n3c4ccccc4c4cccc(-c5ccc6c7ccccc7n(-c7cc(-c8ccccc8)cc(-c8ccccc8)n7)c6c5)c43)c2)cc1 ; c1ccc(-c2cccc(-n3c4ccccc4c4ccc(-c5cccc6c7ccccc7n(-c7cc(-c8ccccc8)cc(-c8ccccc8)n7)c56)cc43)c2)cc1 ; c1ccc(-c2cc(-c3ccccc3)nc(-n3c4ccccc4c4cc(-c5cccc6c7ccccc7n(-c7ccccc7)c56)ccc43)c2)cc1]

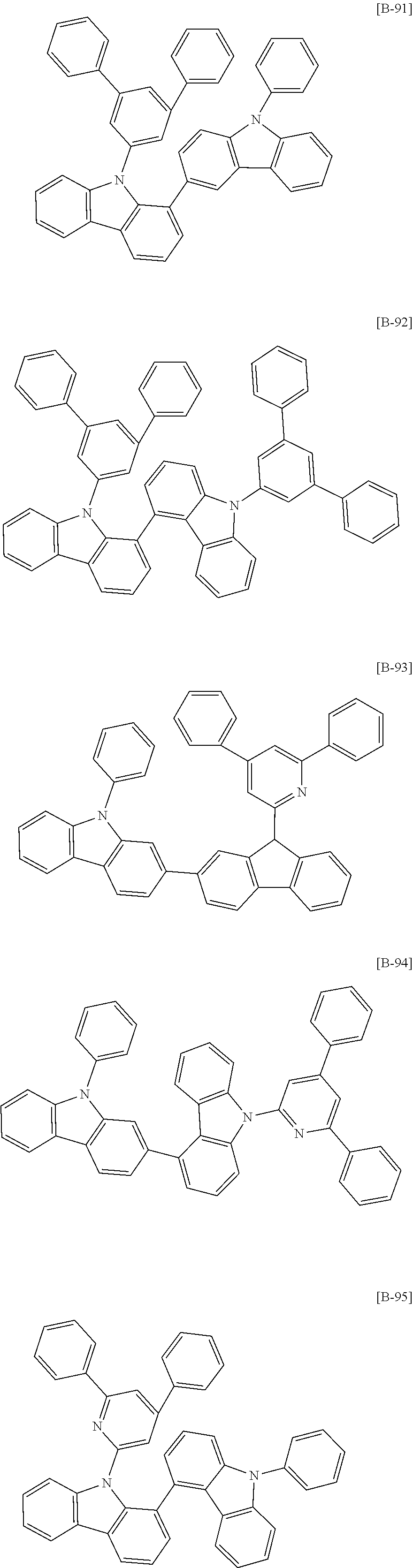 [SMILES: c1ccc(-c2cc(-c3ccccc3)cc(-n3c4ccccc4c4cccc(-c5ccc6c(c5)c5ccccc5n6-c5ccccc5)c43)c2)cc1 ; c1ccc(-c2cc(-c3ccccc3)cc(-n3c4ccccc4c4c(-c5cccc6c7ccccc7n(-c7cc(-c8ccccc8)cc(-c8ccccc8)c7)c56)cccc43)c2)cc1 ; c1ccc(-c2cc(-c3ccccc3)nc(C3c4ccccc4-c4ccc(-c5ccc6c7ccccc7n(-c7ccccc7)c6c5)cc43)c2)cc1 ; c1ccc(-c2cc(-c3ccccc3)nc(-n3c4ccccc4c4c(-c5ccc6c7ccccc7n(-c7ccccc7)c6c5)cccc43)c2)cc1 ; c1ccc(-c2cc(-c3ccccc3)nc(-n3c4ccccc4c4cccc(-c5cccc6c5c5ccccc5n6-c5ccccc5)c43)c2)cc1]

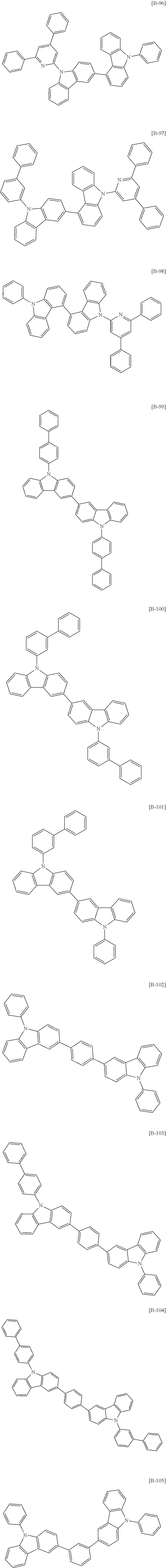

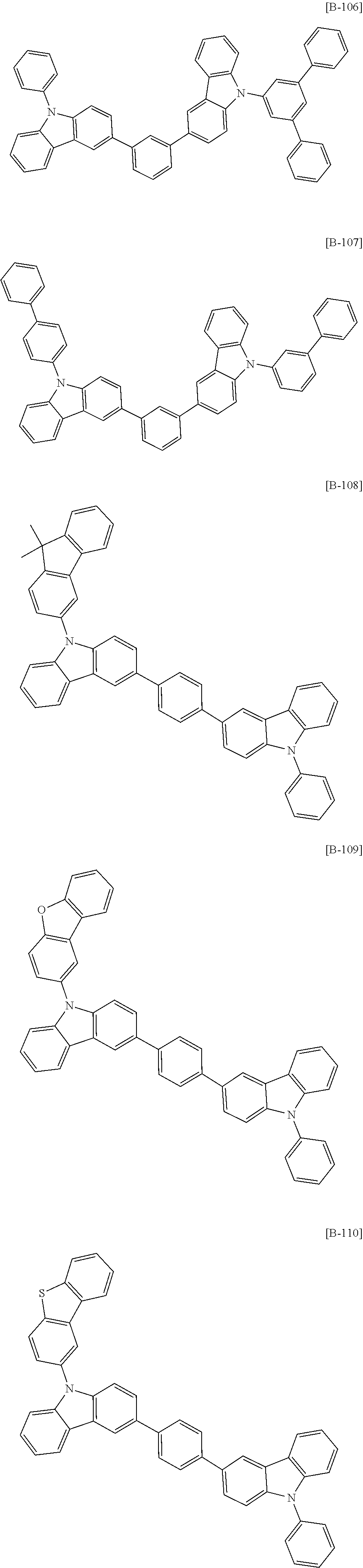 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccc(-c6ccc7c(c6)c6ccccc6n7-c6ccccc6)cc5)ccc43)ccc21 ; c1ccc(-n2c3ccccc3c3cc(-c4ccc(-c5ccc6c(c5)c5ccccc5n6-c5ccc6oc7ccccc7c6c5)cc4)ccc32)cc1 ; c1ccc(-n2c3ccccc3c3cc(-c4ccc(-c5ccc6c(c5)c5ccccc5n6-c5ccc6sc7ccccc7c6c5)cc4)ccc32)cc1]

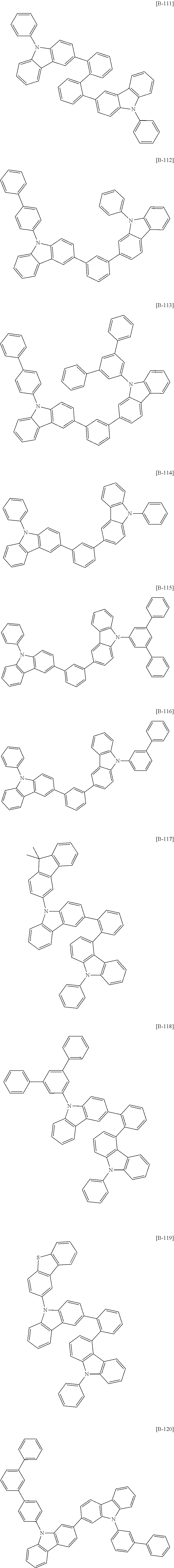

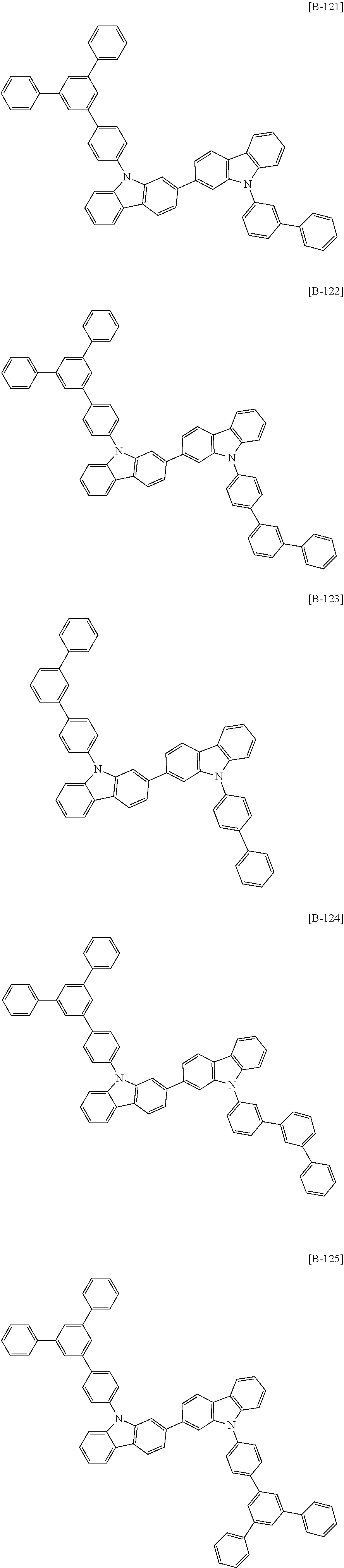

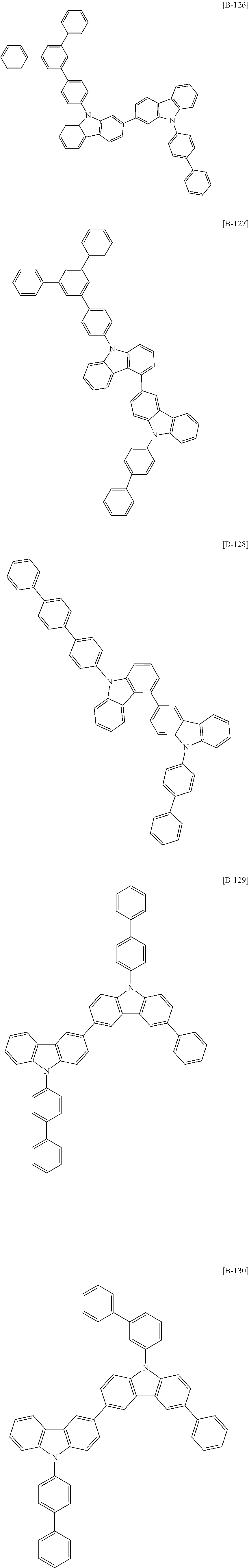

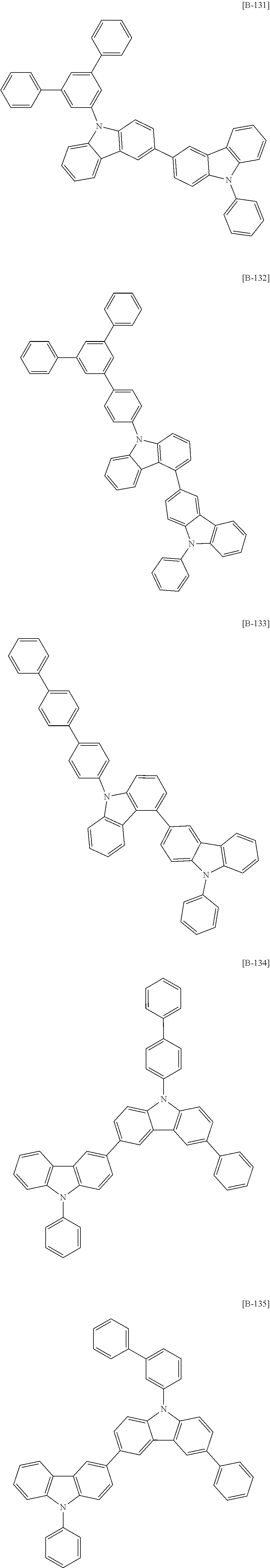 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1]

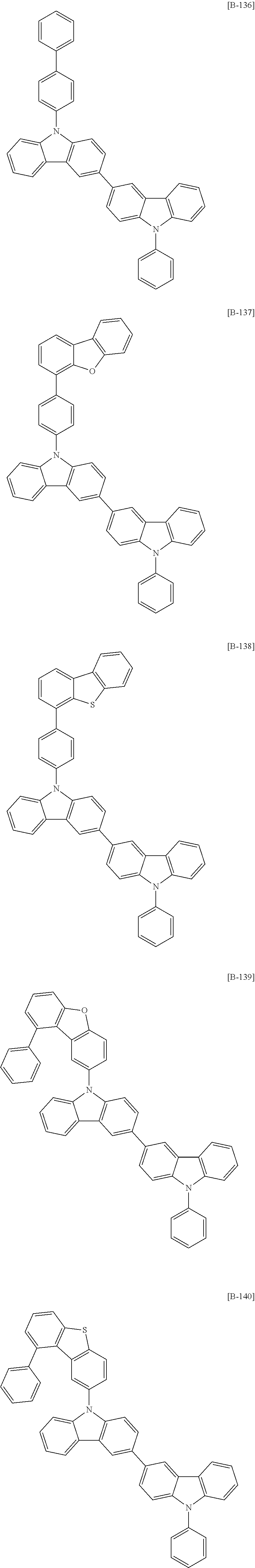 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1 ; c1ccc(-c2cccc3oc4ccc(-n5c6ccccc6c6cc(-c7ccc8c(c7)c7ccccc7n8-c7ccccc7)ccc65)cc4c23)cc1 ; c1ccc(-c2cccc3sc4ccc(-n5c6ccccc6c6cc(-c7ccc8c(c7)c7ccccc7n8-c7ccccc7)ccc65)cc4c23)cc1]

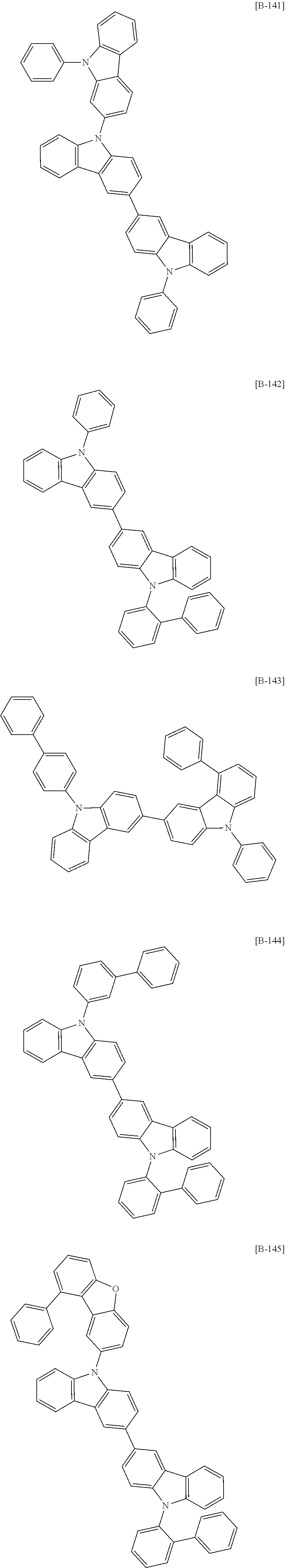 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccccc2-n2c3ccccc3c3cc(-c4ccc5c(c4)c4ccccc4n5-c4ccc5oc6cccc(-c7ccccc7)c6c5c4)ccc32)cc1]

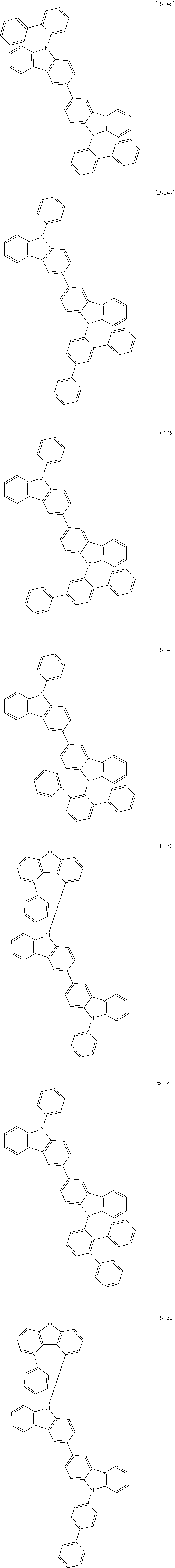

Additionally, examples of Compound B-1 to Compound B-152 listed in Group 2 in which at least one hydrogen is replaced with deuterium are given below, but are not limited thereto.

[chemical structure]

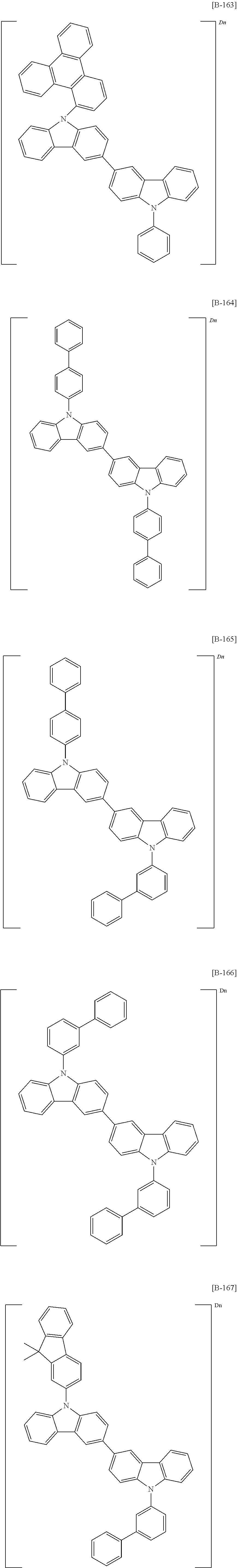

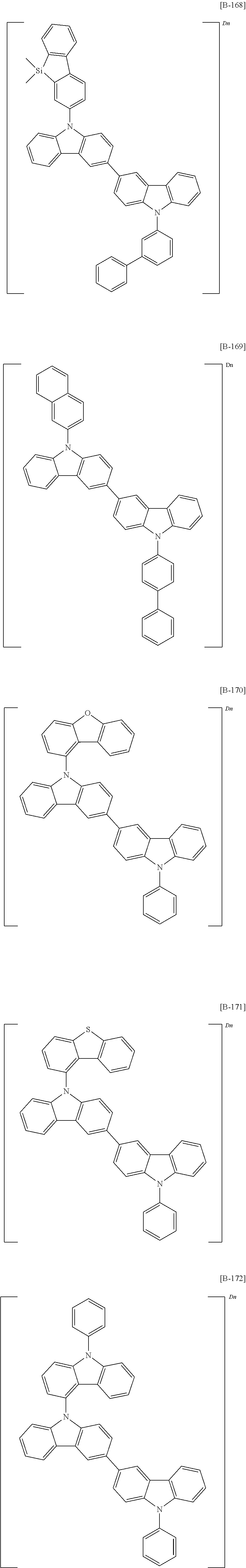

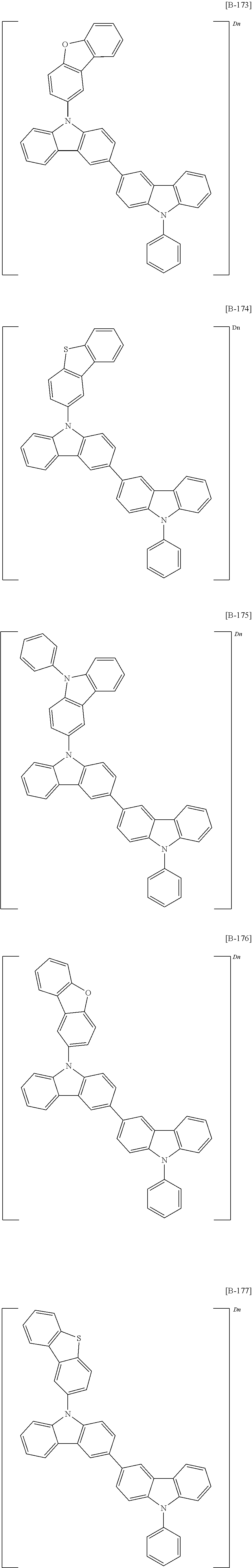

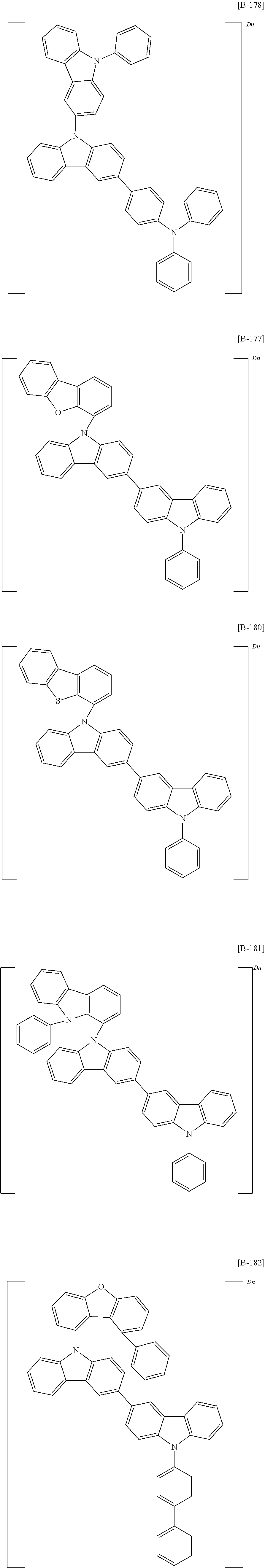

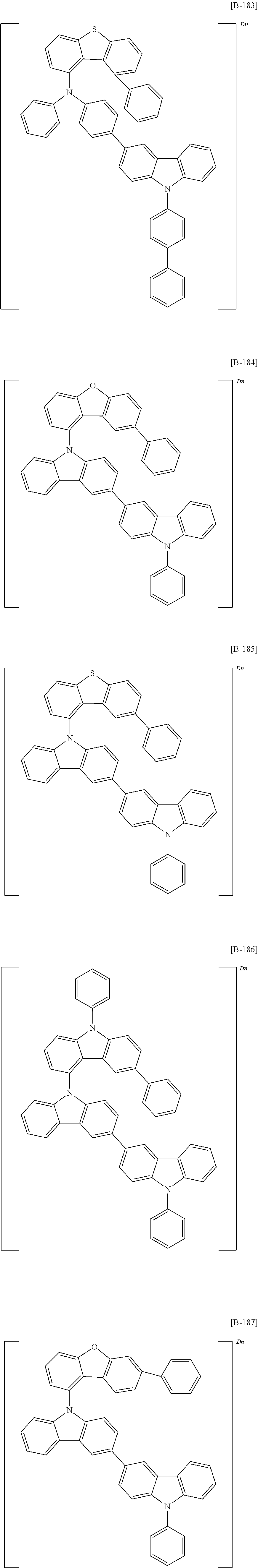

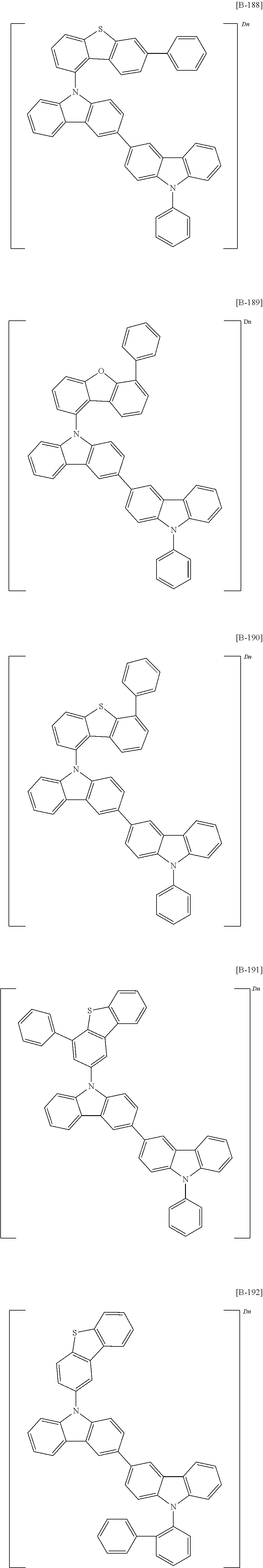

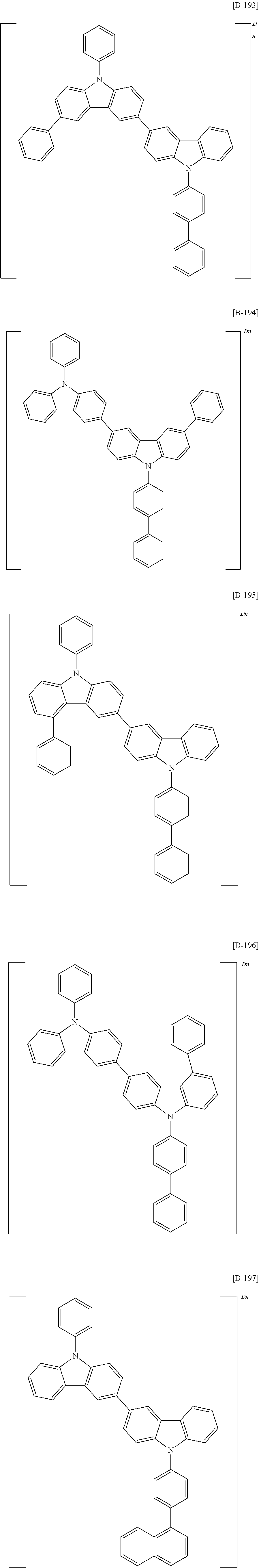

(Dn refers to the number of deuterium substitutions and indicates a structure substituted with one or more deuteriums)

The most specific structures for Compound B-153 to Compound B-197 of Group 2 are presented below as examples according to the position and substitution rate of deuterium substitution, and are not the intention to limit the scope of rights to compounds not listed below.

The scope of the present invention is determined by the claims, and when deuterium is substituted, it is not limited to the compounds exemplified below, and the deuterium substitution position, deuterium substitution rate, etc. may include all changeable ranges within the range of Compound B-1 to Compound B-197.

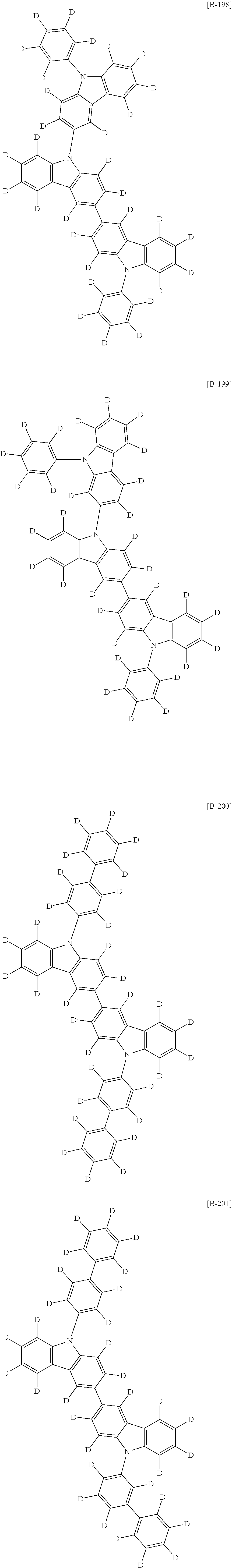

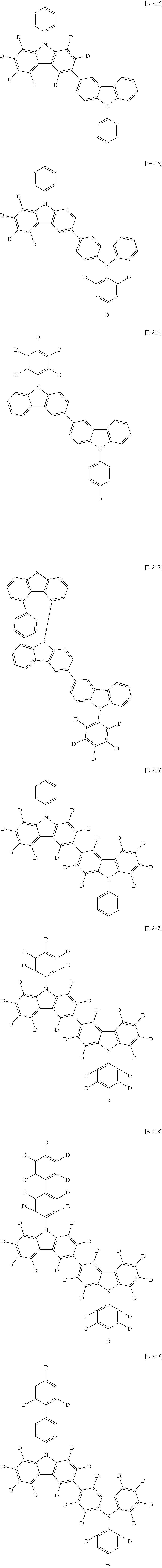

[chemical structure]

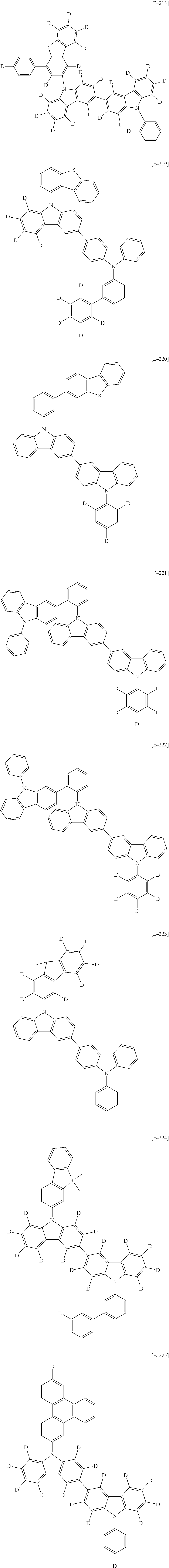

[chemical structure]

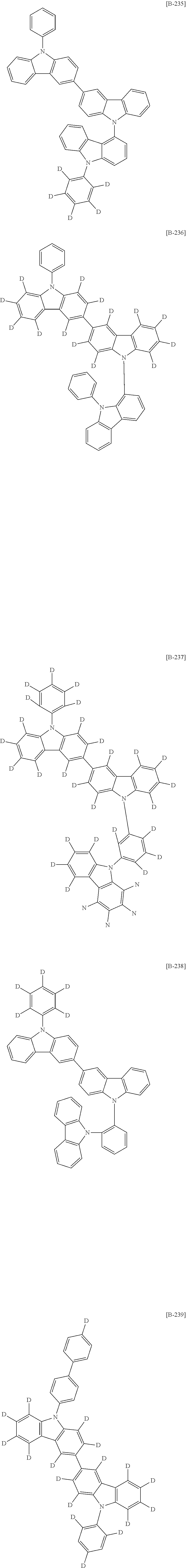

[chemical structure]

[chemical structure]

[chemical structure]

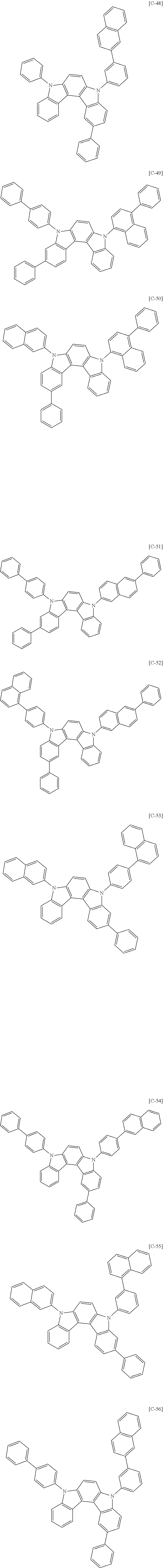

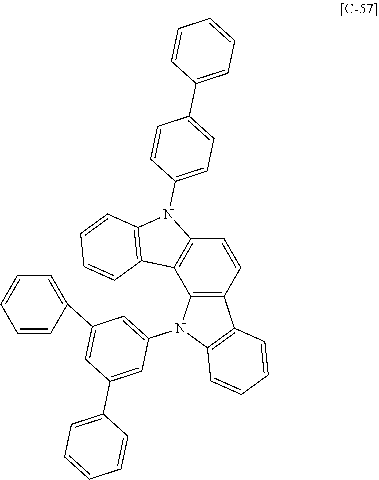 [SMILES: c1ccc(-c2ccc(-n3c4ccccc4c4c3ccc3c5ccccc5n(-c5cc(-c6ccccc6)cc(-c6ccccc6)c5)c34)cc2)cc1]

Additionally, examples of Compound C-1 to Compound C-57 listed in Group 2 in which at least one hydrogen is replaced with deuterium are given below, but are not limited thereto.

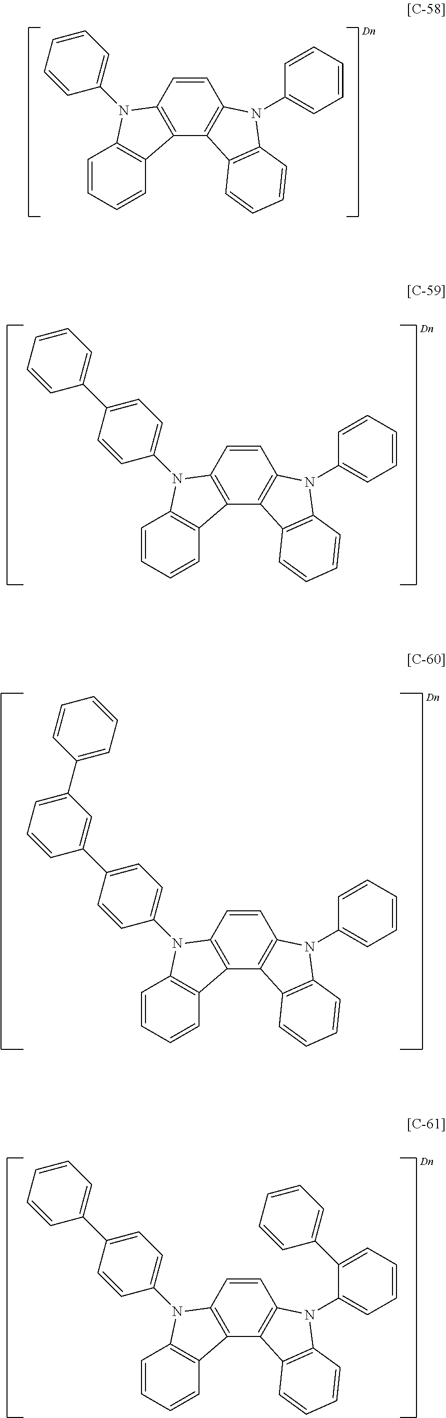

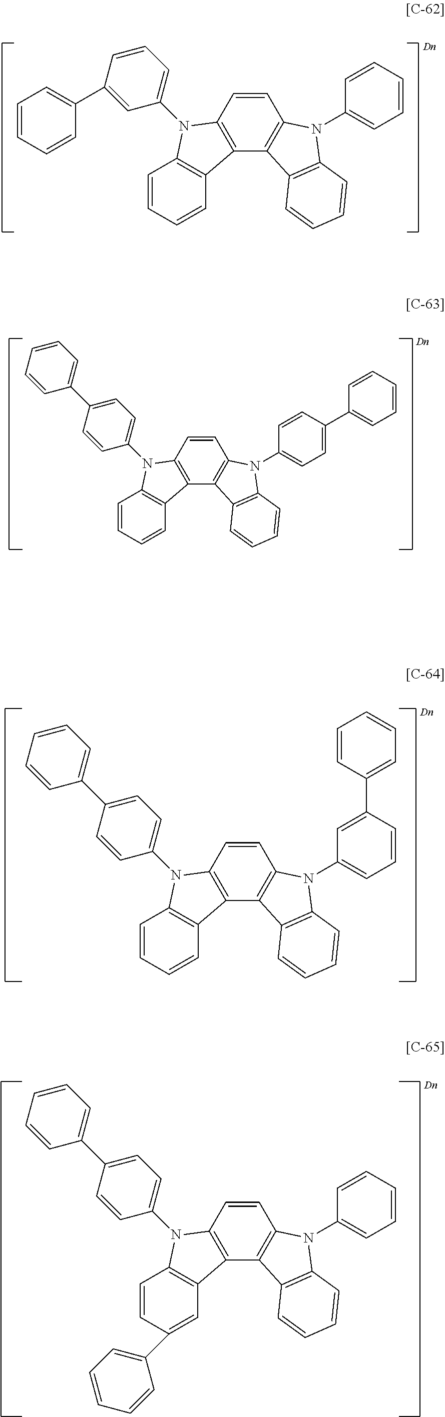

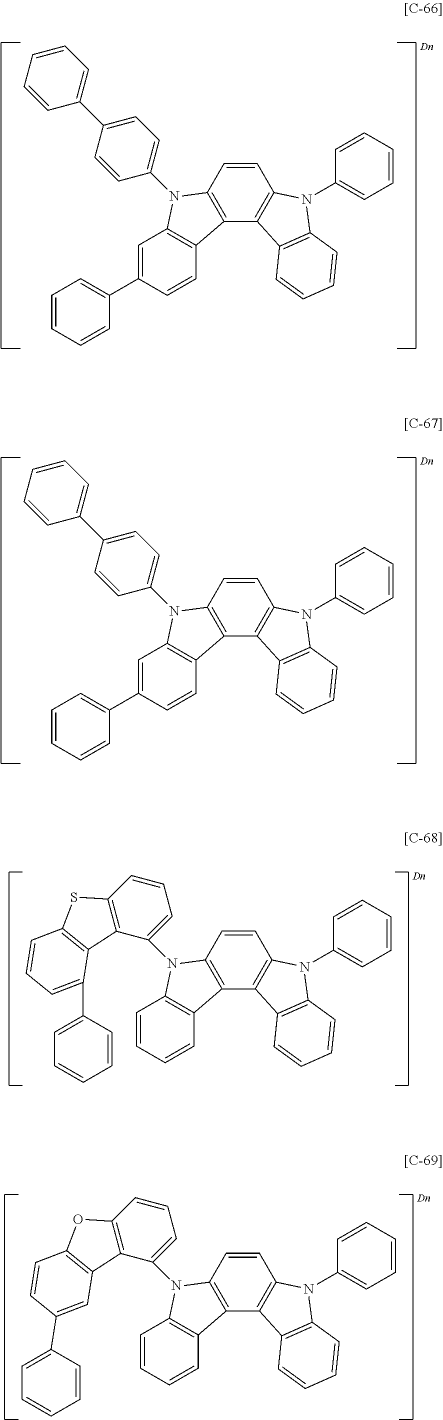

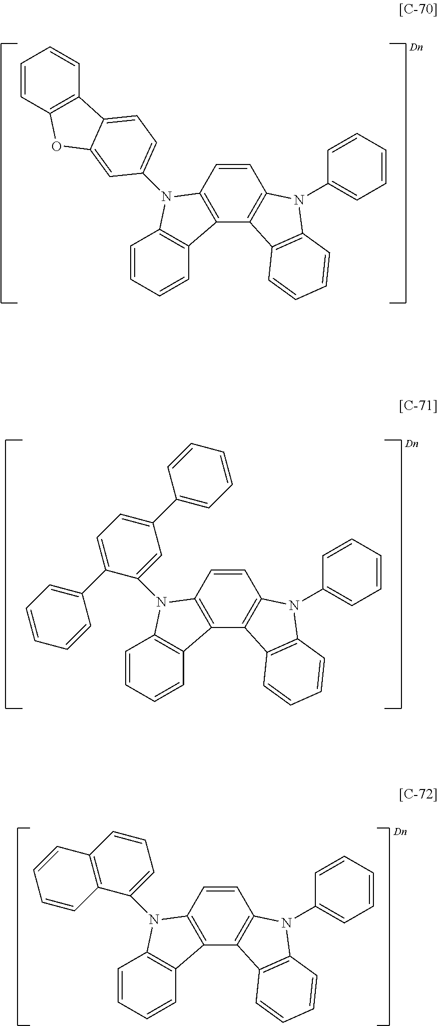

(Dn refers to the number of deuterium substitutions and indicates a structure substituted with one or more deuteriums)

The most specific structures for Compound C-58 to Compound C-72 of Group 2 are presented below as examples according to the position and substitution rate of deuterium substitution, and are not the intention to limit the scope of rights to compounds not listed below.

The scope of the present invention is determined by the claims, and when deuterium is substituted, it is not limited to the compounds exemplified below, and the deuterium substitution position, deuterium substitution rate, etc. may include all changeable ranges within the range of Compound C-58 to Compound C-72.

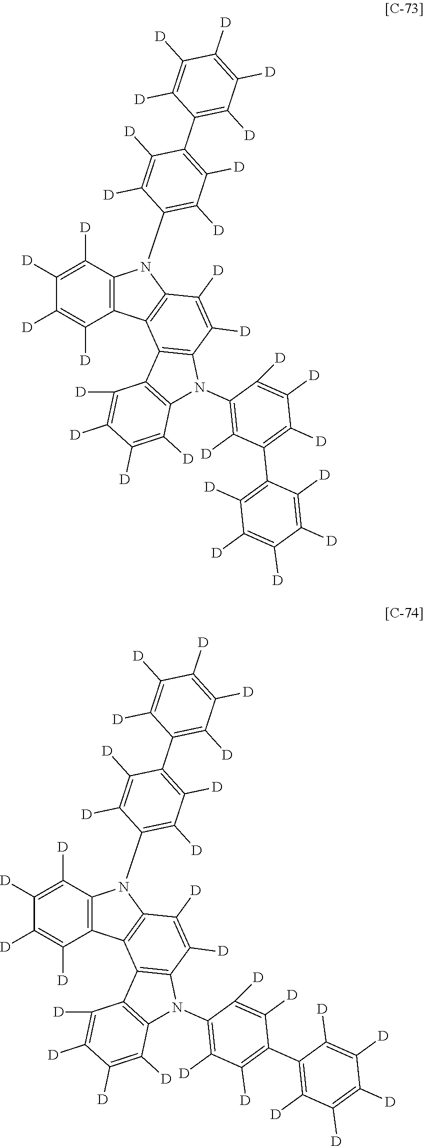 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]]

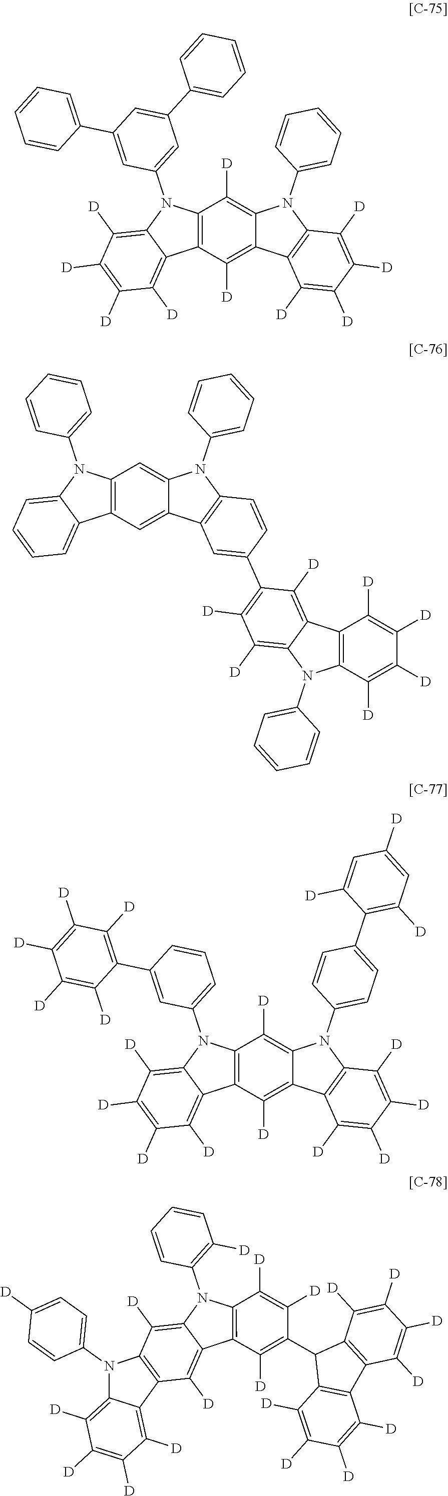

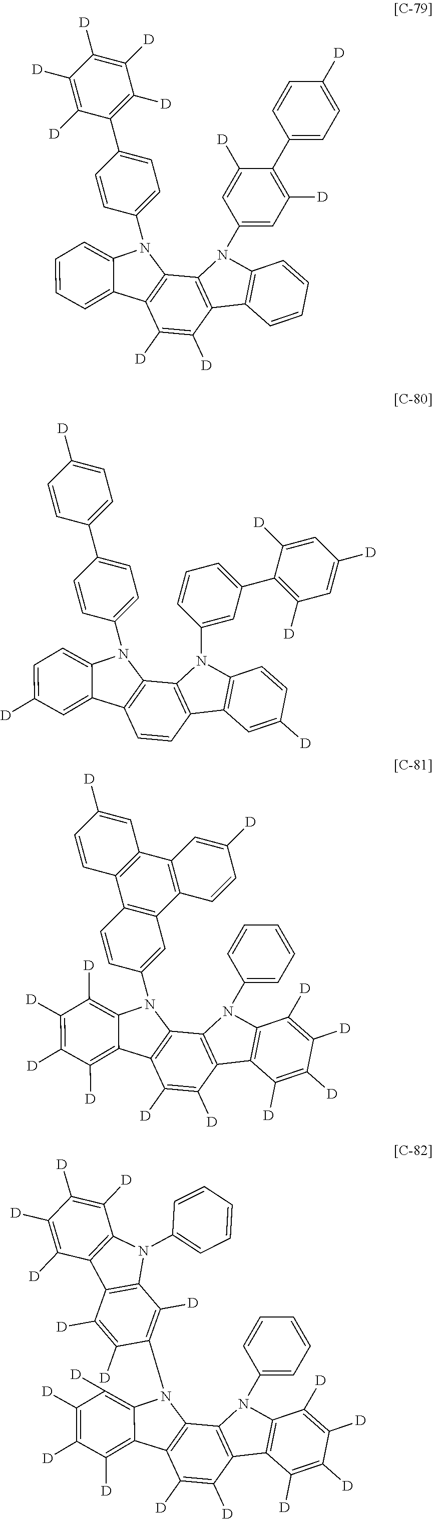

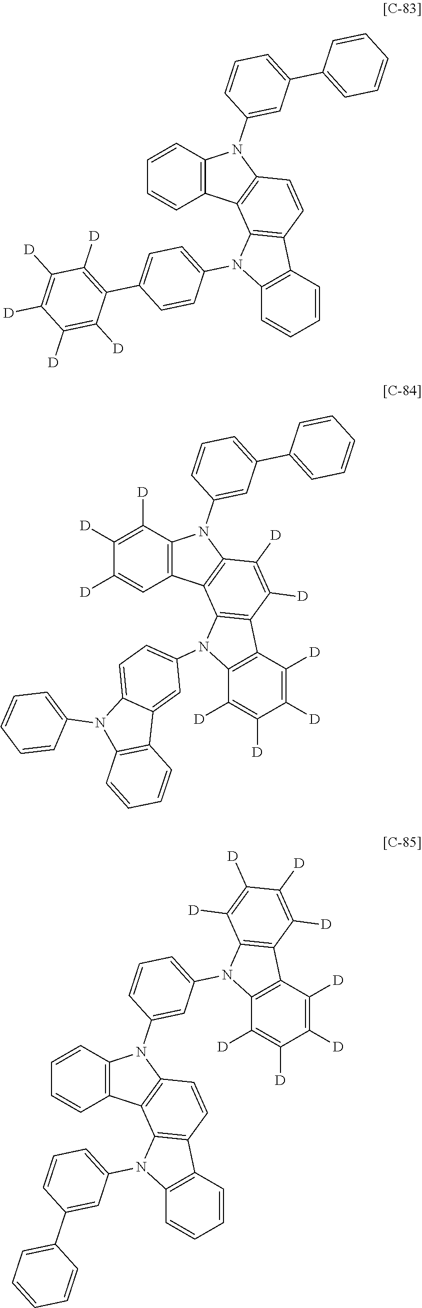 [SMILES: [2H]c1c([2H])c([2H])c(-c2ccc(-n3c4ccccc4c4ccc5c(c6ccccc6n5-c5cccc(-c6ccccc6)c5)c43)cc2)c([2H])c1[2H] ; [2H]c1cc2c3c(c([2H])c([2H])c4c5c([2H])c([2H])c([2H])c([2H])c5n(-c5ccc6c(c5)c5ccccc5n6-c5ccccc5)c43)n(-c3cccc(-c4ccccc4)c3)c2c([2H])c1[2H] ; [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1]

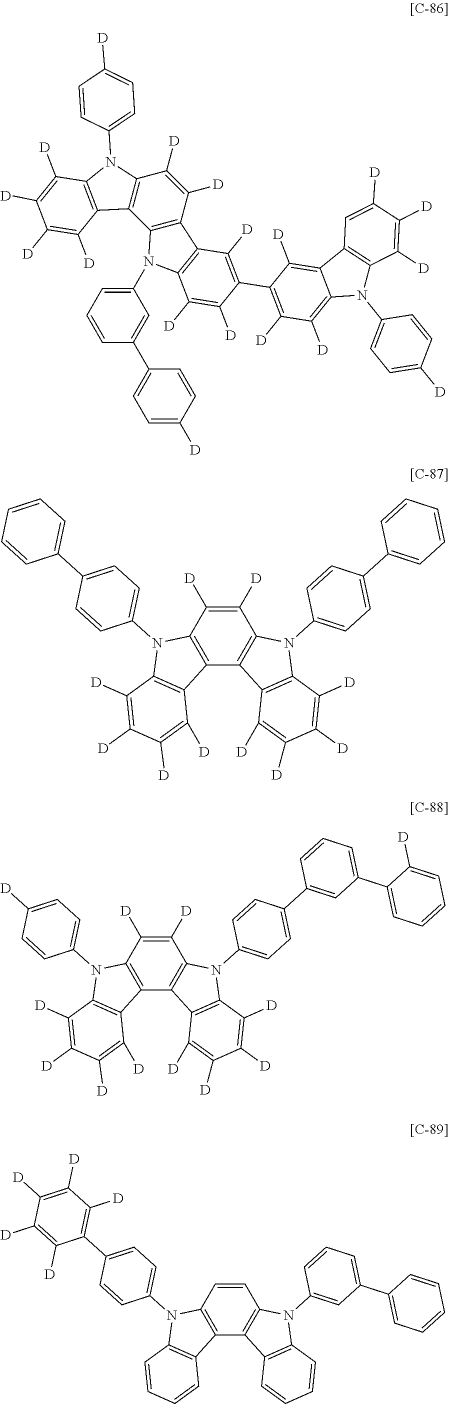

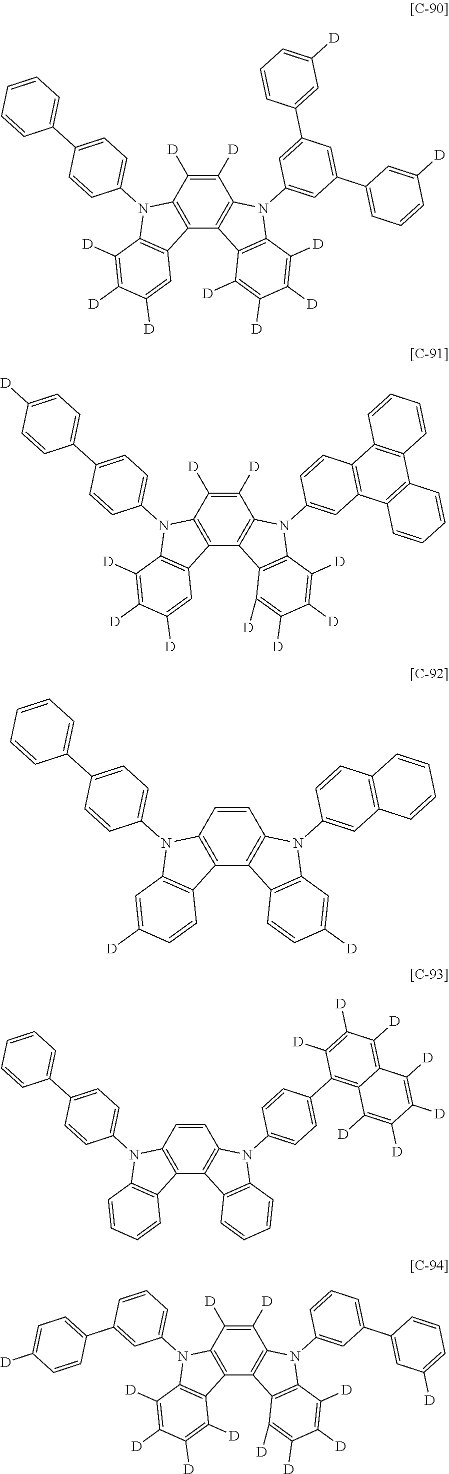

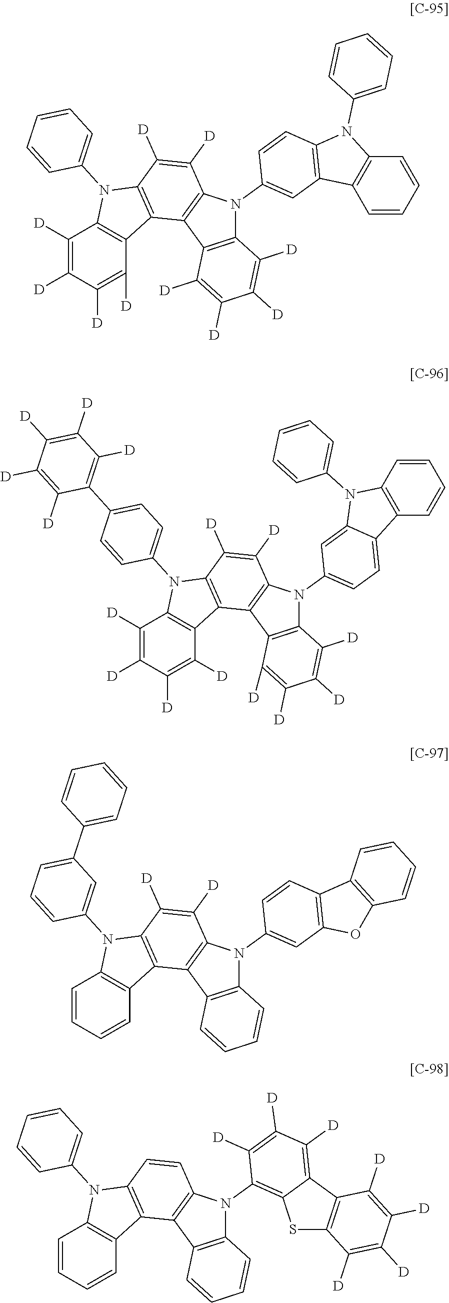 [SMILES: [2H]c1c([2H])c([2H])c2c(c1[2H])c1c3c4c([2H])c([2H])c([2H])c([2H])c4n(-c4ccc5c(c4)c4ccccc4n5-c4ccccc4)c3c([2H])c([2H])c1n2-c1ccccc1 ; [2H]c1c([2H])c([2H])c(-c2ccc(-n3c4c([2H])c([2H])c([2H])c([2H])c4c4c5c6c([2H])c([2H])c([2H])c([2H])c6n(-c6ccc7c8ccccc8n(-c8ccccc8)c7c6)c5c([2H])c([2H])c43)cc2)c([2H])c1[2H] ; [2H]c1c([2H])c2c(c3ccccc3n2-c2ccc3c(c2)oc2ccccc23)c2c3ccccc3n(-c3cccc(-c4ccccc4)c3)c12 ; [2H]c1c([2H])c([2H])c2c(sc3c(-n4c5ccccc5c5c6c7ccccc7n(-c7ccccc7)c6ccc54)c([2H])c([2H])c([2H])c32)c1[2H]]

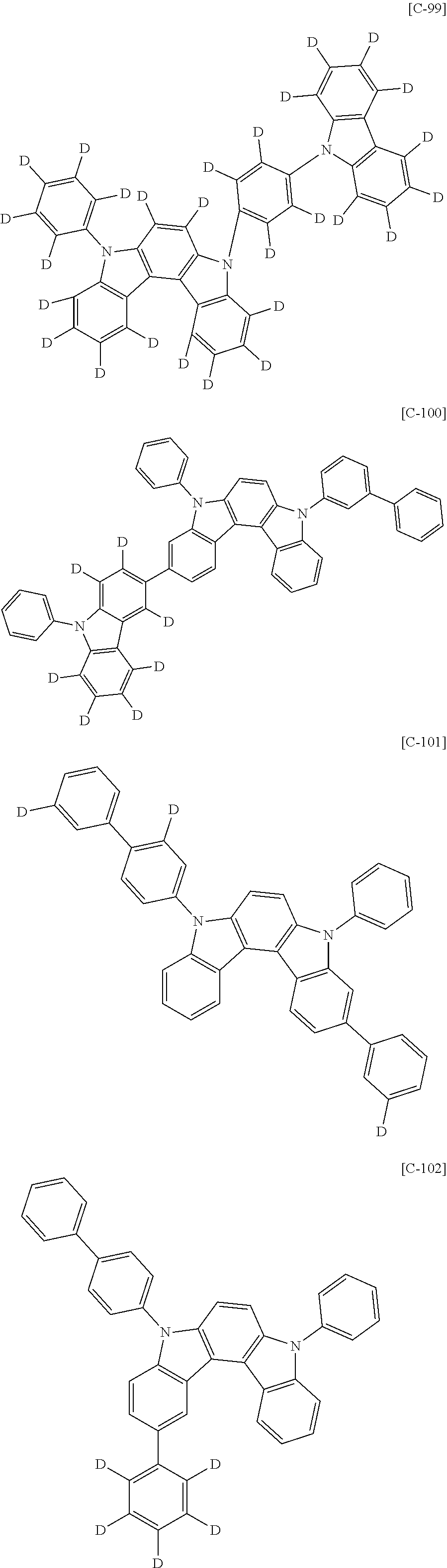

The first compound and the second compound may be included in a weight ratio of, for example, 1:99 to 99:1. Within the above range, efficiency and life-span can be improved by implementing bipolar characteristics by adjusting the appropriate weight ratio using the electron transport ability of the first compound and the hole transport ability of the second compound. Within the above range, they may be included in a weight ratio of, for example, about 10:90 to 90:10, about 20:80 to 80:20, for example about 20:80 to about 70:30, about 20:80 to about 60:40, and about 30:70 to about 60:40. As a specific example, they may be included in a weight ratio of 40:60, 50:50, or 60:40.

In addition to the first compound and the second compound, one or more additional compounds may be included.

The compound for an organic optoelectronic device or the composition for an organic optoelectronic device may be a composition further including a dopant.

The dopant may be, for example, a phosphorescent dopant, such as a red, green, or blue phosphorescent dopant, and may be, for example, a red or green phosphorescent dopant.

The dopant is a material mixed with the compound or composition for an organic optoelectronic device in a small amount to cause light emission and may be generally a material such as a metal complex that emits light by multiple excitation into a triplet or more. The dopant may be, for example an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

L⁶MX [Chemical Formula Z]

In Chemical Formula Z, M is a metal, and L⁶ and X are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof, and L⁶ and X may be, for example a bidentate ligand.

Examples of the ligands represented by L⁶ and X may be selected from the chemical formulas listed in Group A, but are not limited thereto.

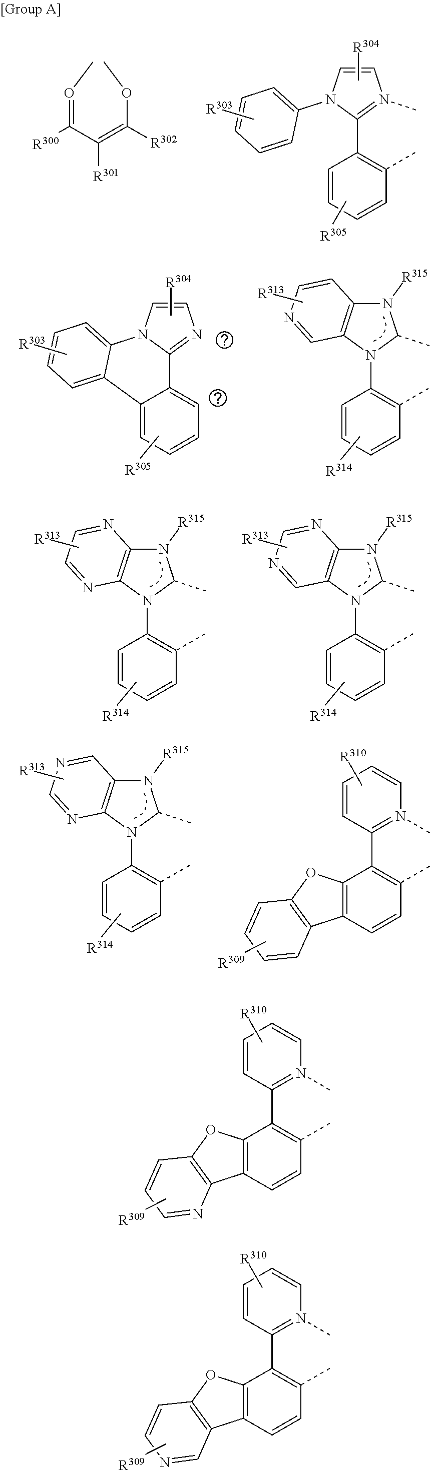

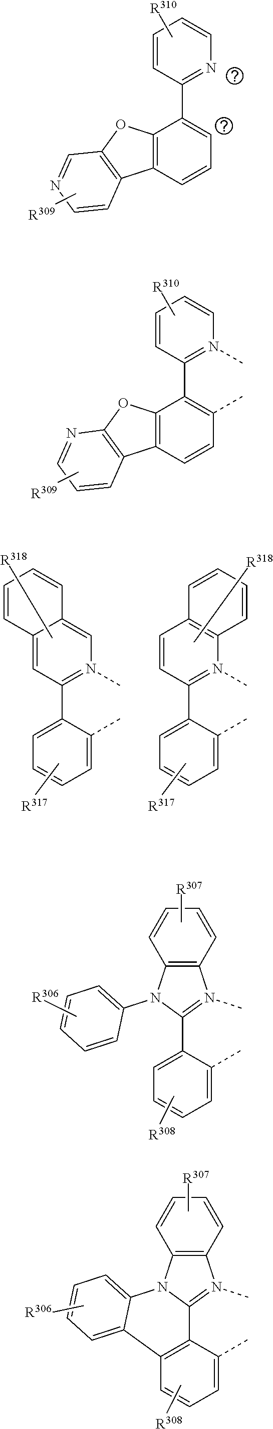

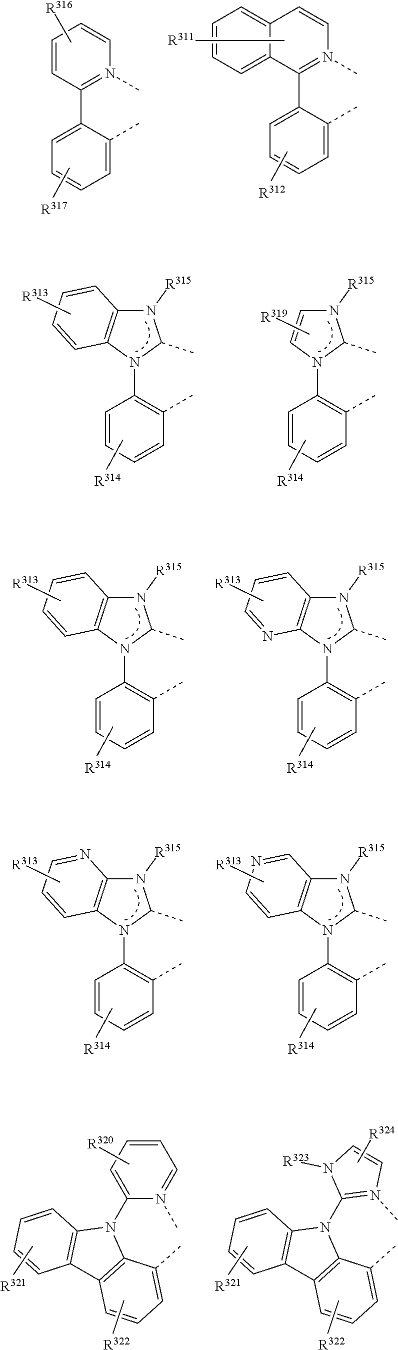

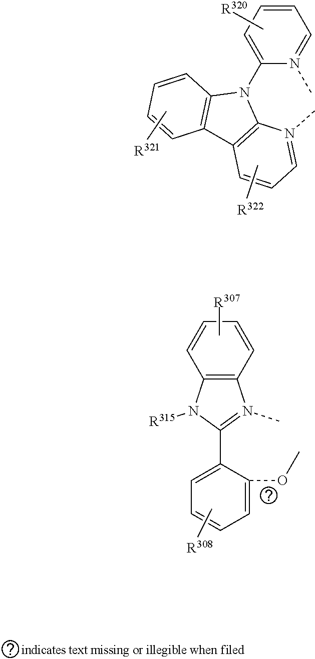

In Group A,
R³⁰⁰ to R³⁰² are each independently hydrogen, deuterium, a C1 to C30 alkyl group that is substituted or unsubstituted with a halogen, a C6 to C30 aryl group that is substituted or unsubstituted with a C1 to C30 alkyl, or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

In an embodiment, the dopant may be an iridium complex, and may be represented by one of Chemical Formula 5-1 or Chemical Formula 5-2.

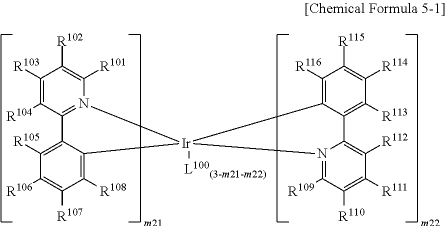

In Chemical Formula 5-1,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a substituted or unsubstituted C1 to C6 alkyl group,
at least one of R¹⁰¹ to R¹¹⁶ is a functional group represented by Chemical Formula V-1,
L¹⁰⁰ is a bidentate ligand of a monovalent anion, and is a ligand that coordinates to iridium through a lone pair of carbons or heteroatoms, and
m21 and m22 are each independently any one of integers of 0 to 3, and m21+m22 is any one of integers of 1 to 3,

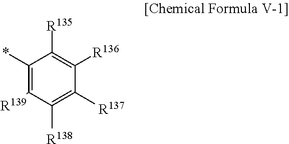

wherein, in Chemical Formula V-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* means a portion linked to a carbon atom.

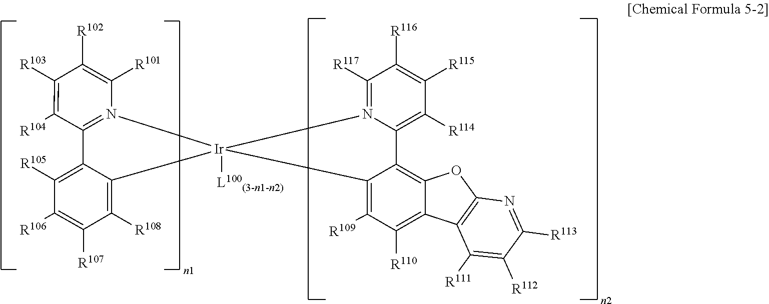

In Chemical Formula 5-2,
R¹⁰¹ to R¹¹⁷ are each independently hydrogen, deuterium, a substituted or unsubstituted C1 to C10 alkyl group, a substituted or unsubstituted C6 to C20 aryl group, or —Si R¹³³R¹³⁴R¹³⁵,
R¹³³ to R¹³⁵ are each independently a substituted or unsubstituted C1 to C6 alkyl group,
L¹⁰⁰ is a bidentate ligand of a monovalent anion, and is a ligand that coordinates to iridium through a lone pair of carbons or heteroatoms, and
n1 and n2 are each independently any one of integers of 0 to 3, and n1+n2 is any one of integers of 1 to 3.

In another embodiment, the dopant may be a platinum complex, for example represented by Chemical Formula Z-1.

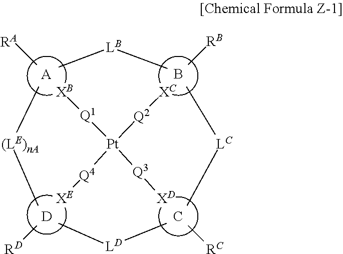

In Chemical Formula Z-1, rings A, B, C, and D are each independently a 5-membered or 6-membered carbocyclic or heterocyclic ring;
R^{A}, R^{B}, R^{C}, and R^{D} are each independently mono-, di-, tri-, or tetra-substitution, or unsubstitution;
L^{B}, L^{C}, and L^{D} are each independently a direct bond, BR, NR, PR, O, S, Se, C═O, S═O, SO₂, CRR′, SiRR′, GeRR′, or a combination thereof,
when nA is 1, L^{E} may be a direct bond, BR, NR, PR, O, S, Se, C═O, S═O, SO₂, CRR′, SiRR′, GeRR′, or a combination thereof, and when nA is 0, L^{E} does not exist;
R^{A}, R^{B}, R^{C}, R^{D}, R, and R′ are each independently hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The platinum complex may be represented, for example, by Chemical Formula 6-1 or Chemical Formula 6-2.

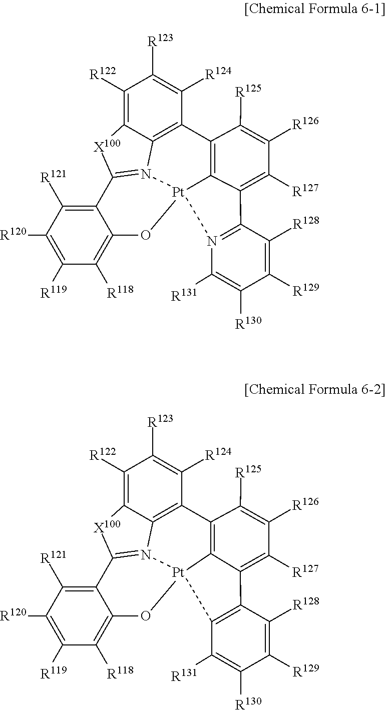

In Chemical Formula 6-1 and Chemical Formula 6-2,
X¹⁰⁰ is selected from O, S, and NR¹³²,
R¹¹⁸ to R¹³² are each independently hydrogen, deuterium, a substituted or unsubstituted C1 to C10 alkyl group, a substituted or unsubstituted C6 to C20 aryl group, or —SiR¹³³R¹³⁴R¹³⁵,
R¹³³ to R¹³⁵ are each independently a substituted or unsubstituted C1 to C6 alkyl group, at least one of R¹¹⁸ to R¹³² is —SiR¹³³R¹³⁴R¹³⁵ or a tert-butyl group, and
R¹³³ to R¹³⁵ are each independently a substituted or unsubstituted C1 to C6 alkyl group.

Hereinafter, an organic optoelectronic device including the aforementioned compound for an organic optoelectronic device or composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

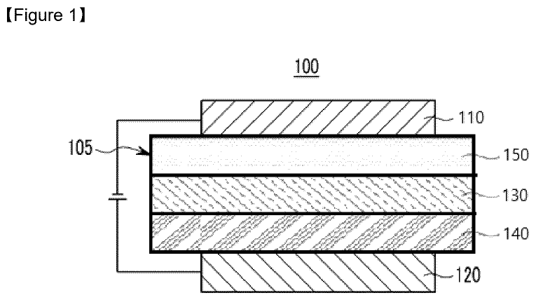

FIG. 1 is a cross-sectional view showing an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof, a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer.

The cathode 110 may include a metal such as magnesium, calcium, sodium, potassium, titanium, indium, yttrium, lithium, gadolinium, aluminum, silver, tin, lead, cesium, barium, or an alloy thereof, a multilayer structure material such as LiF/Al, LiO₂/Al, LiF/Ca, LiF/Al, and BaF₂/Ca, but is not limited thereto.

The organic layer 105 may include the aforementioned compound for an organic optoelectronic device or composition for an organic optoelectronic device.

The organic layer 105 may include a light emitting layer 130 and the light emitting layer 130 may include the aforementioned compound for an organic optoelectronic device or composition for an organic optoelectronic device.

The composition for an organic optoelectronic device further including a dopant may be, for example, a green light emitting composition.

The light emitting layer 130 may include, for example, each of the aforementioned compound for an organic optoelectronic device or composition for an organic optoelectronic device as a phosphorescent host.

The organic layer may further include a charge transport region in addition to the light emitting layer.

The charge transport region may be, for example, a hole transport region 140.

The hole transport region 140 may further increase hole injection and/or hole mobility between the anode 120 and the light emitting layer 130 and block electrons.

Specifically, the hole transport region 140 may include a hole transport layer between the anode 120 and the light emitting layer 130, and a hole transport auxiliary layer between the light emitting layer 130 and the hole transport layer, and at least one of the compounds of Group B may be included in at least one of the hole transport layer and the hole transport auxiliary layer.

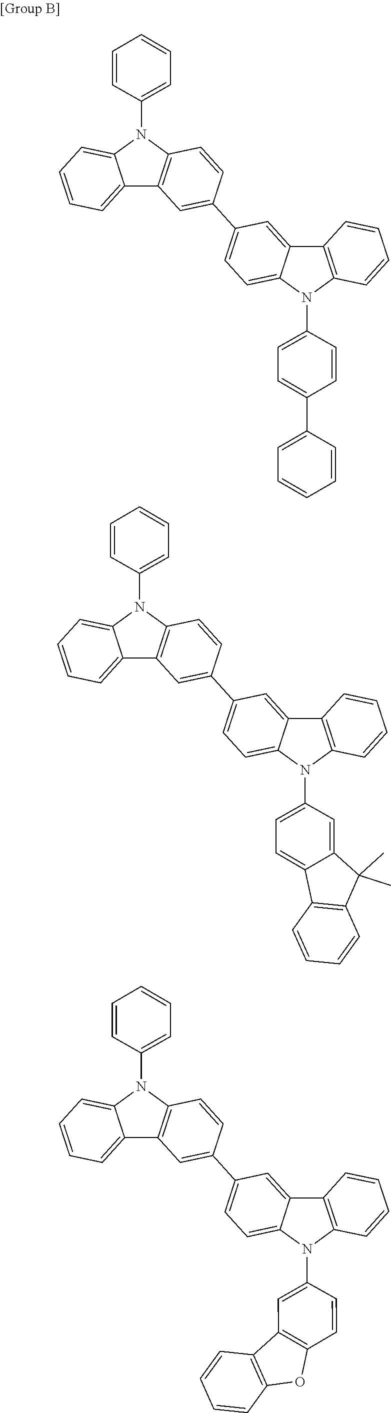 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

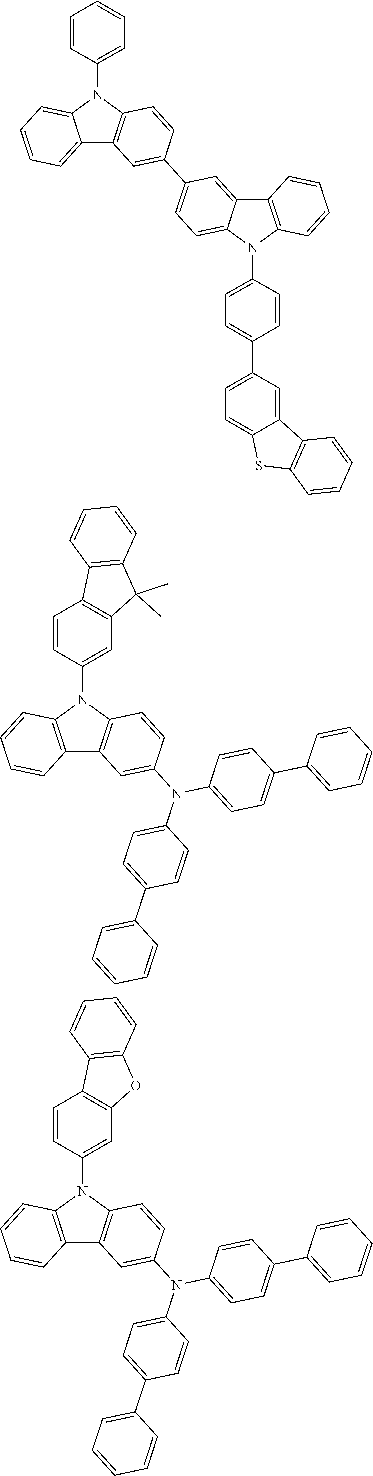

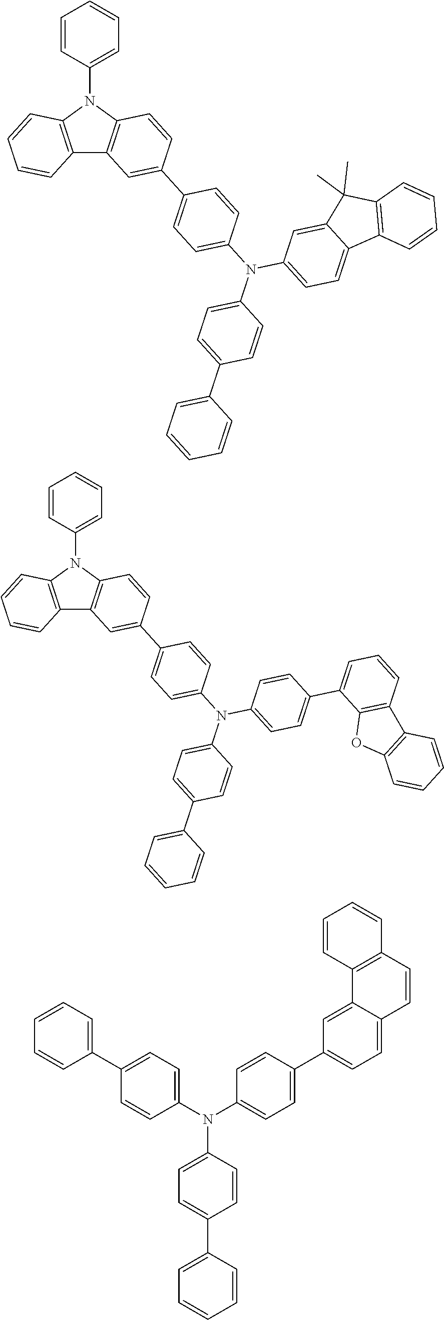 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1]

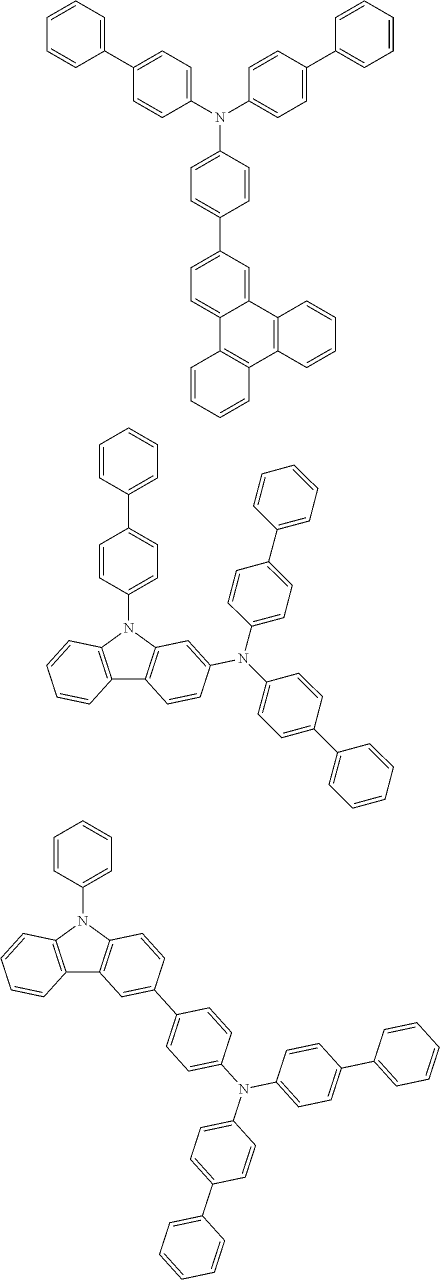 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

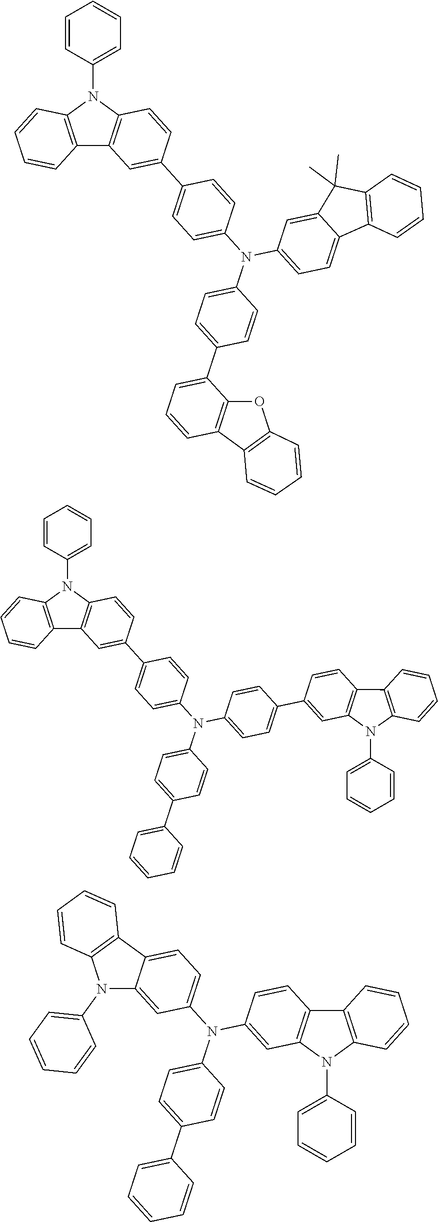

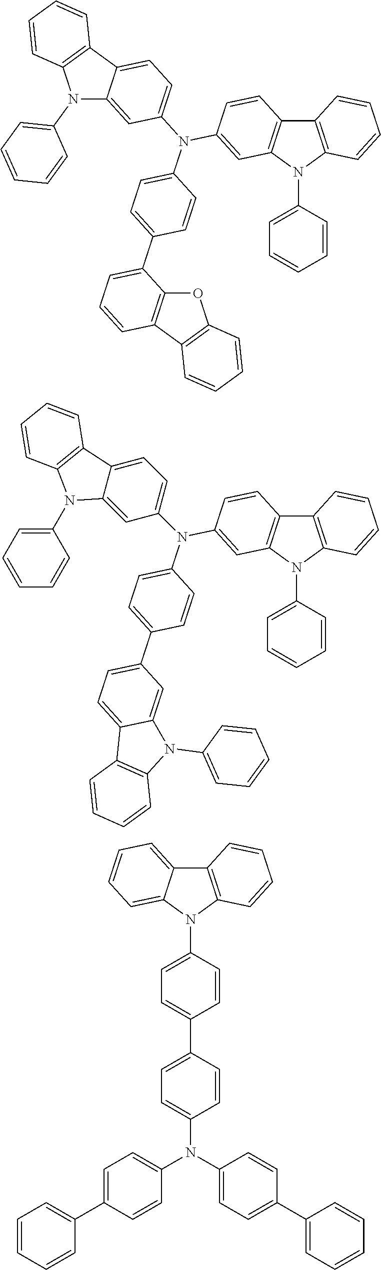

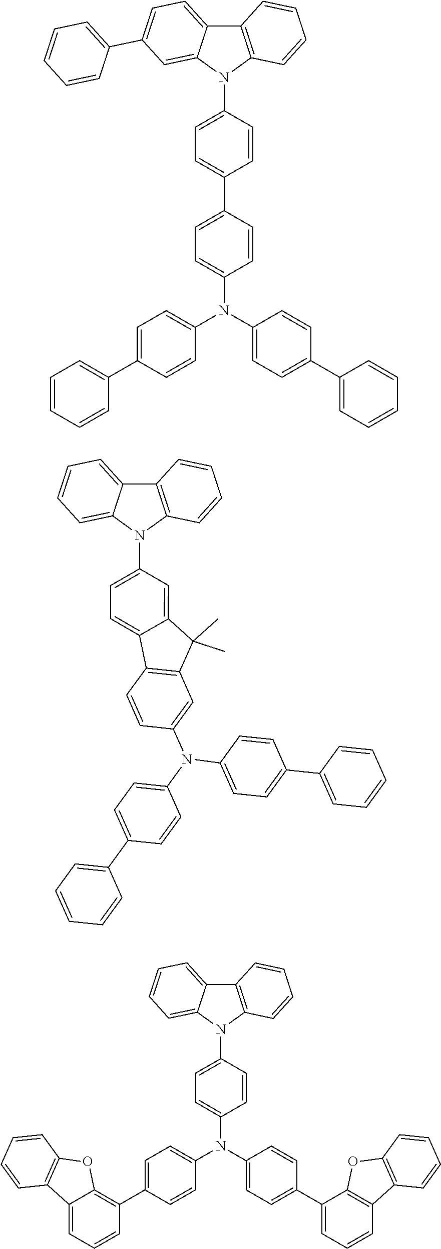

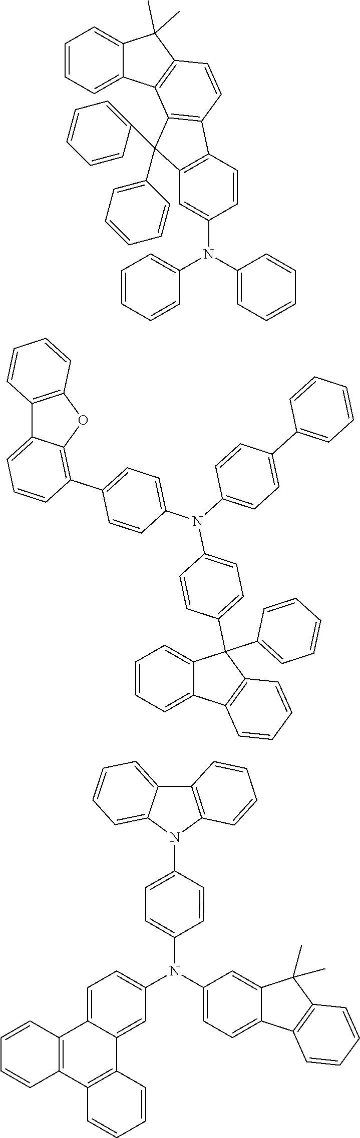

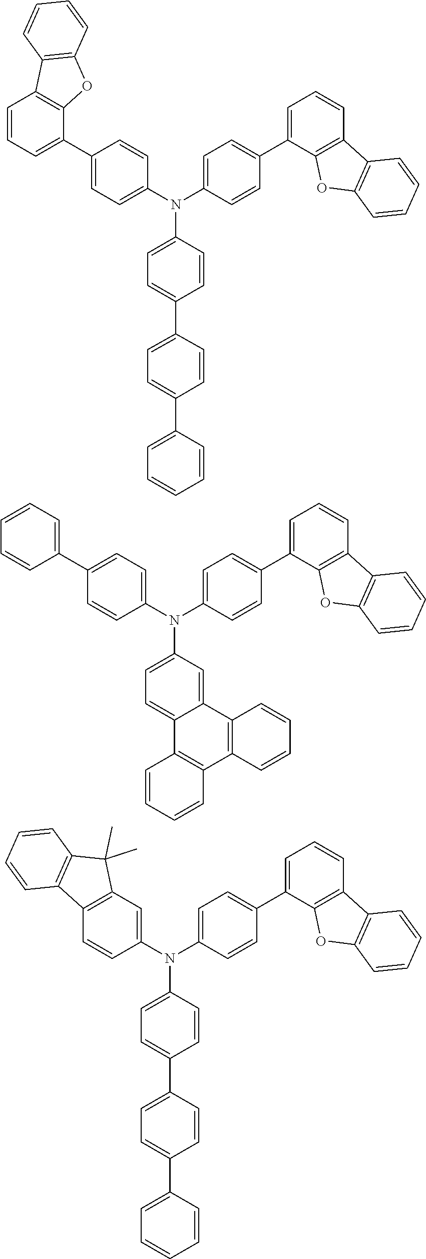

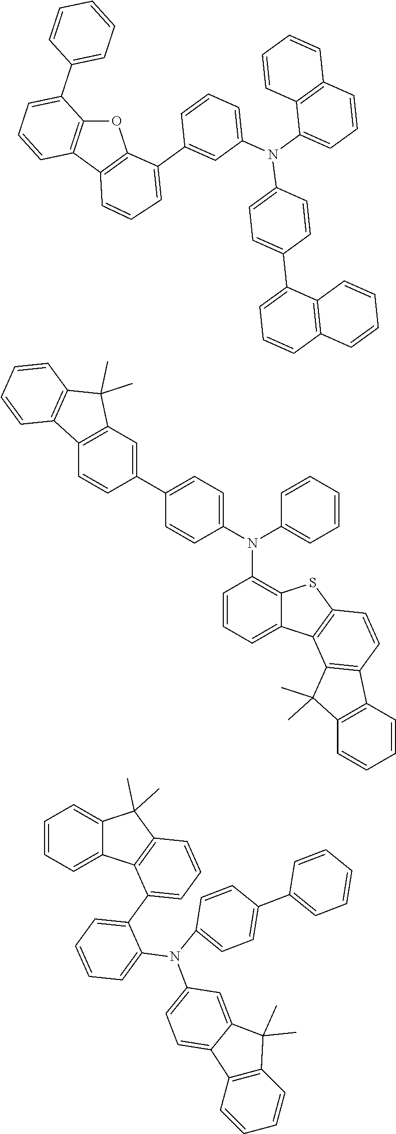 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

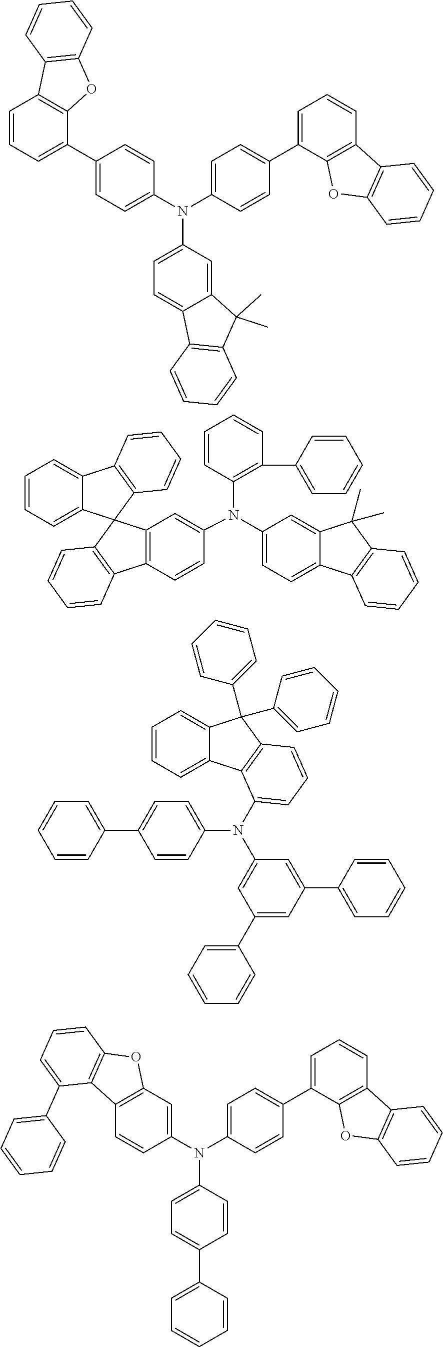

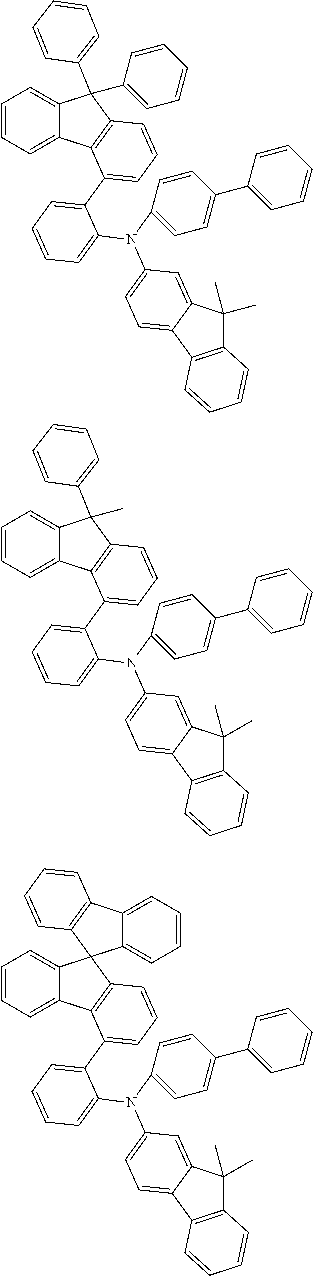 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

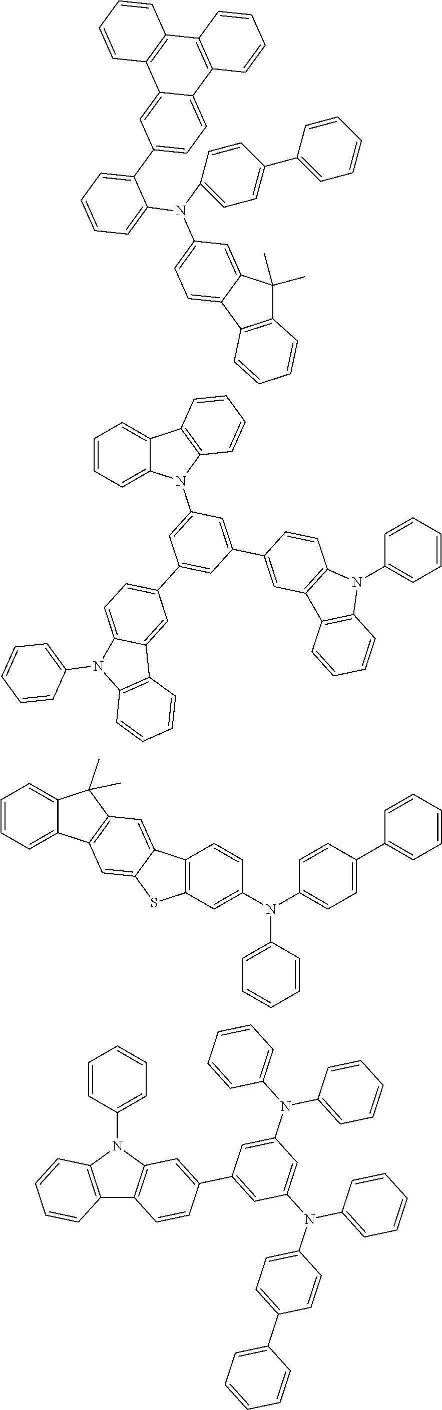

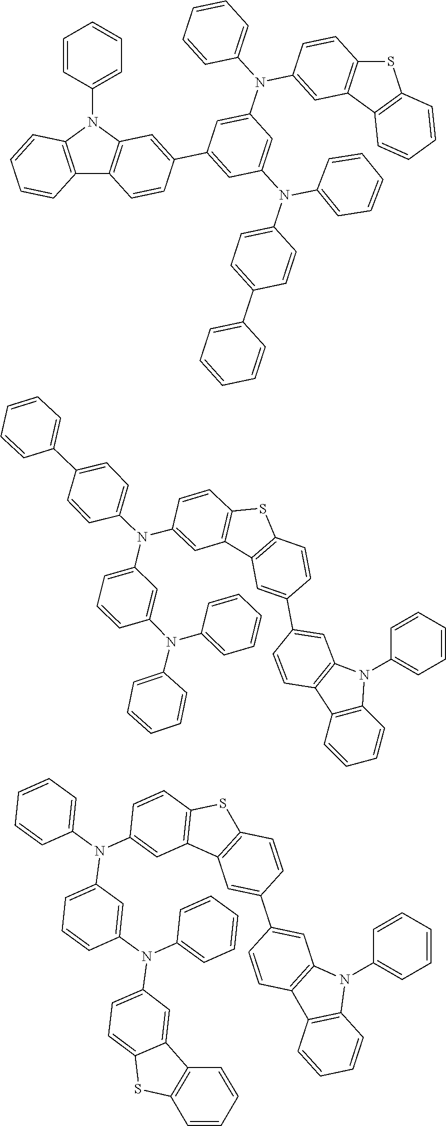 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1]

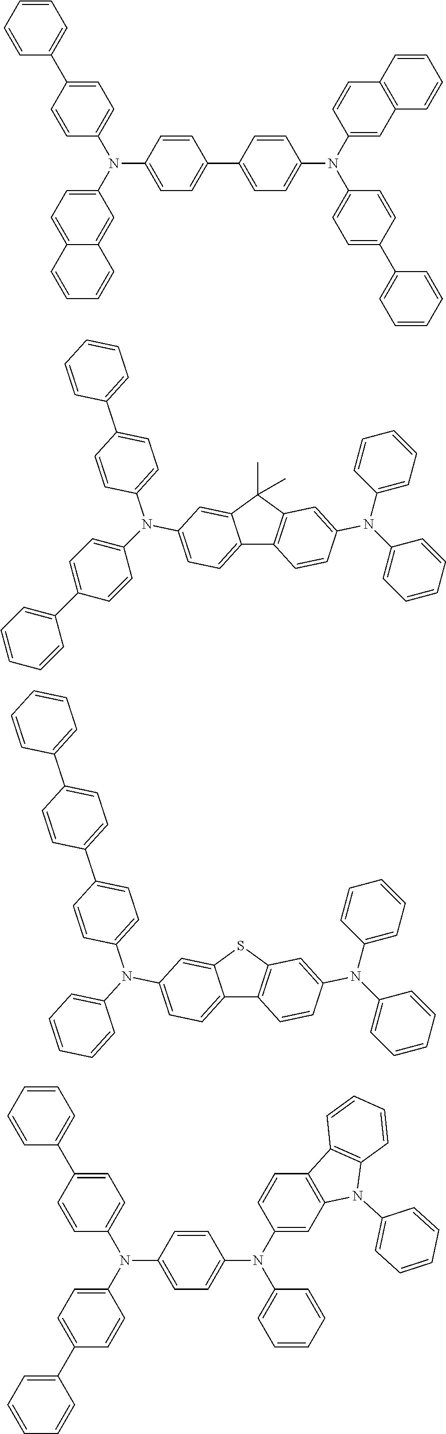

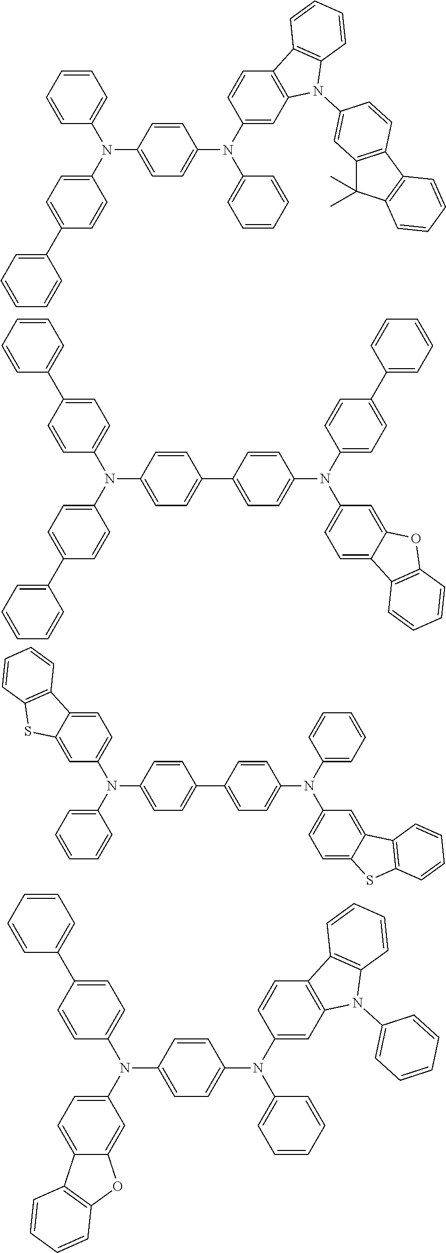

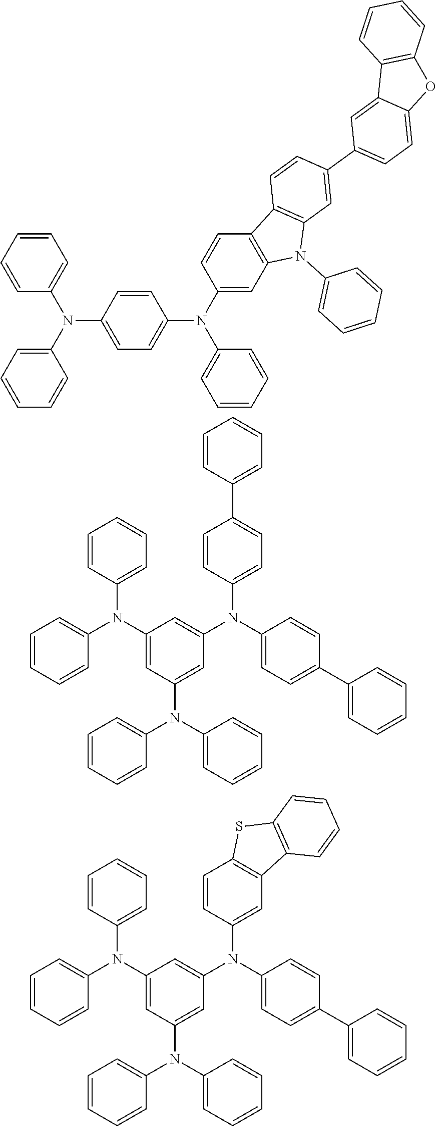

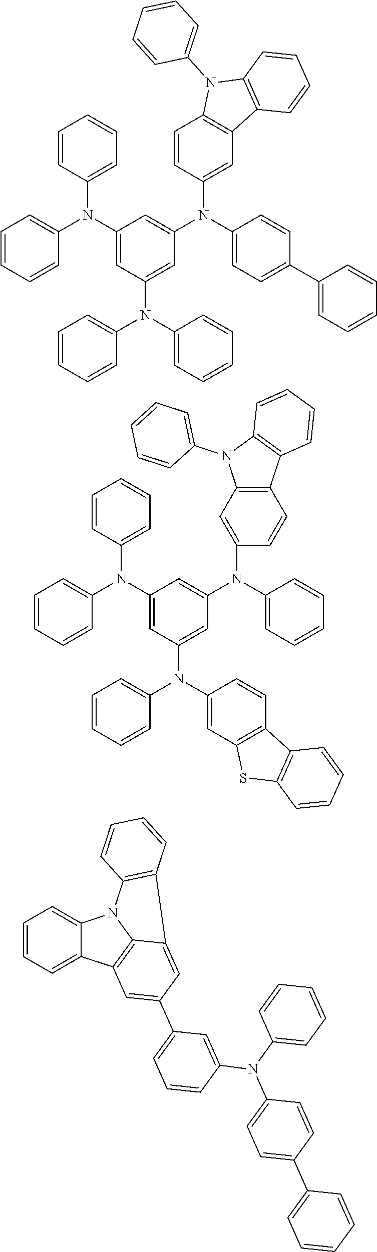

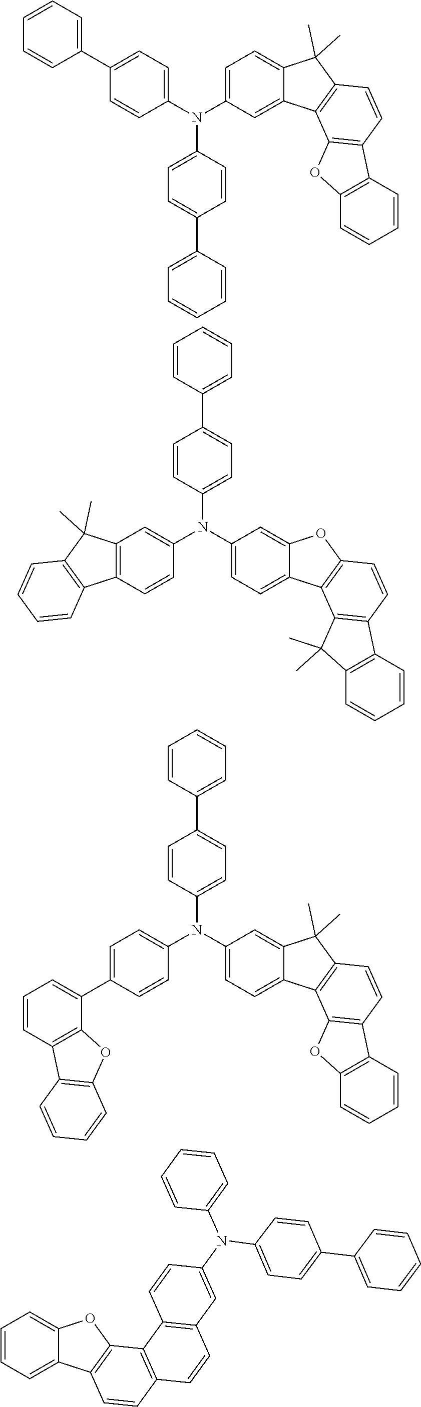

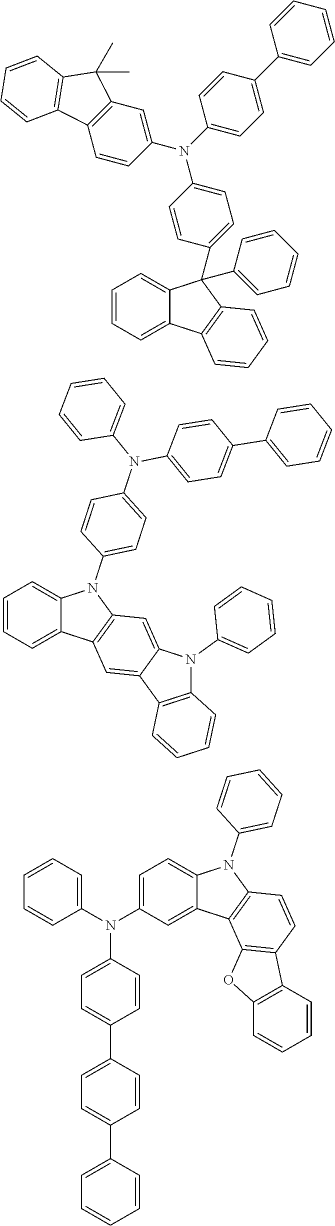

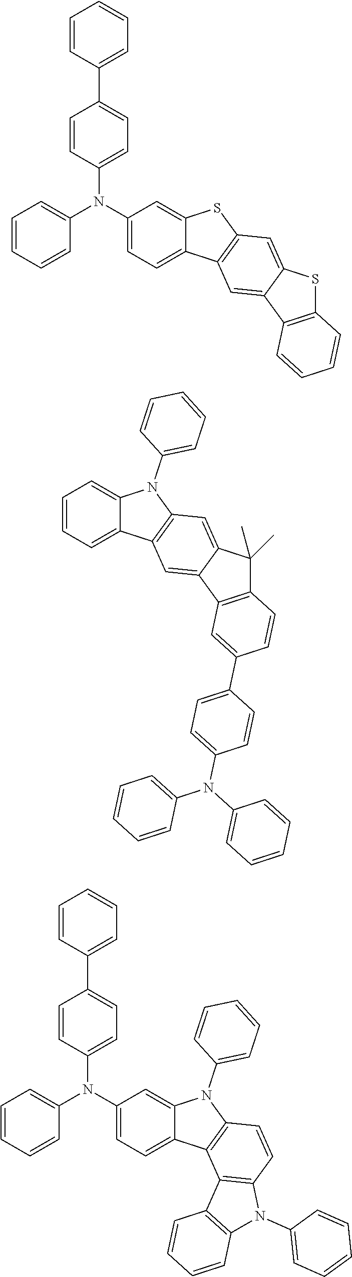 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

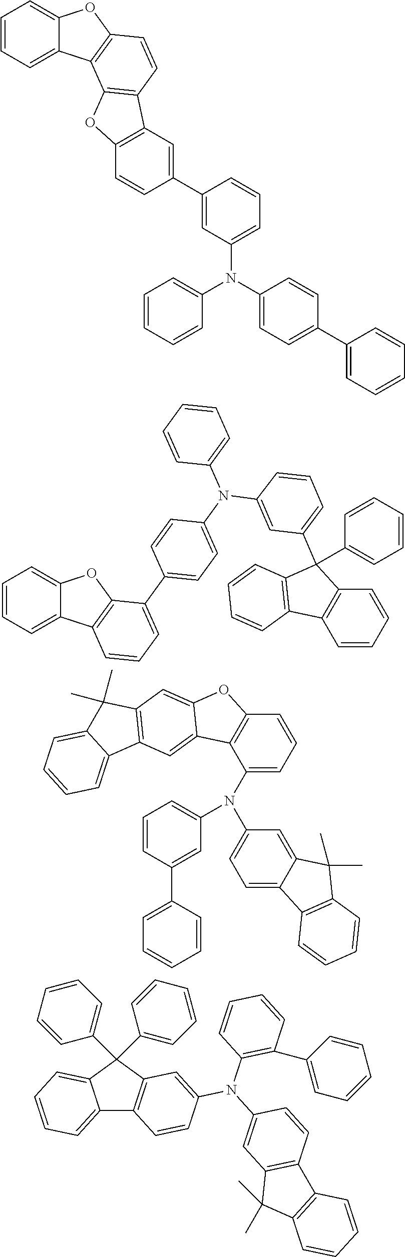

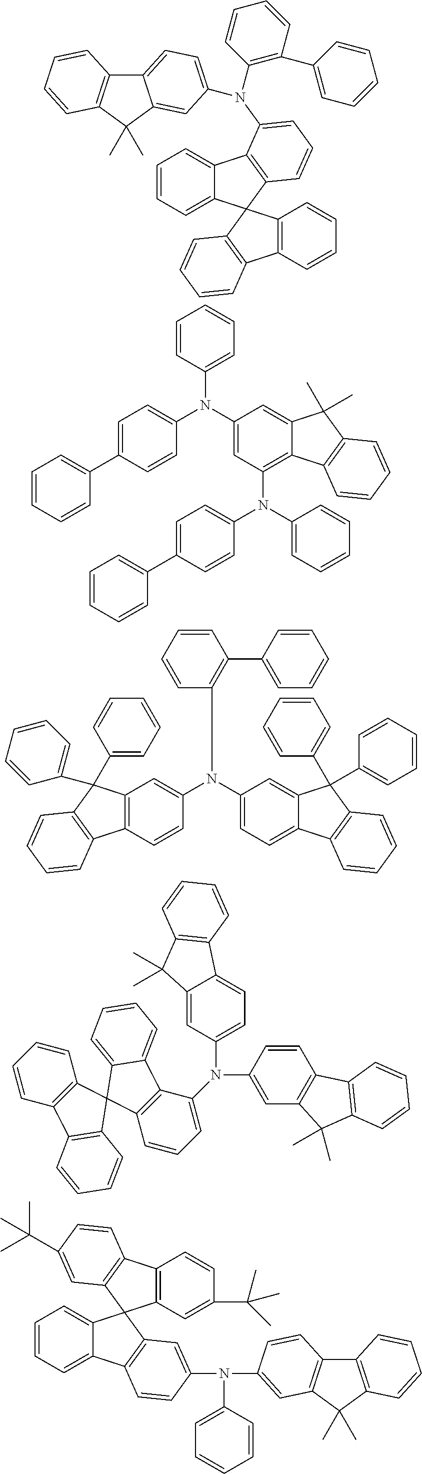

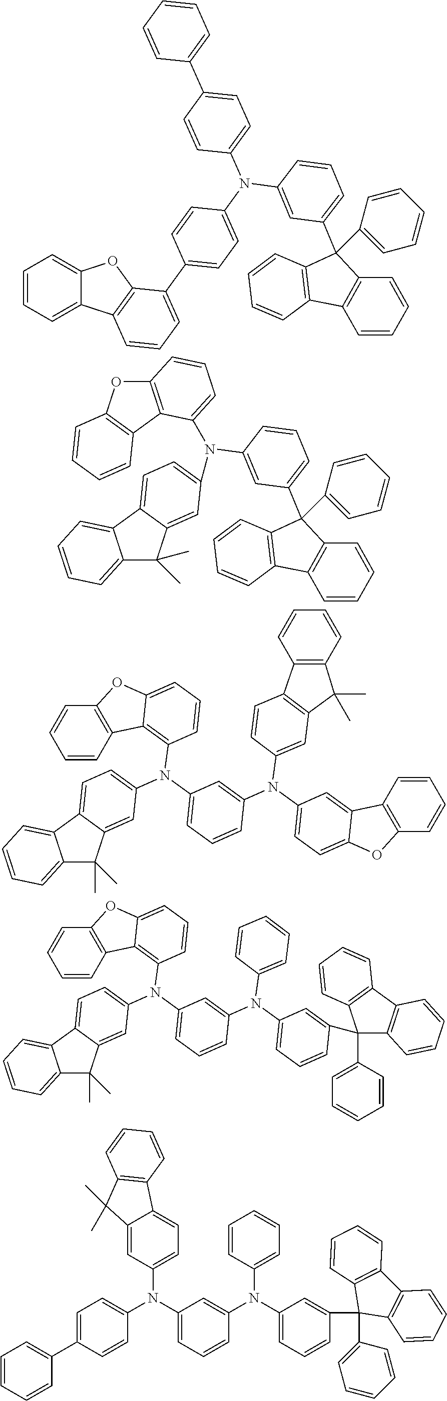

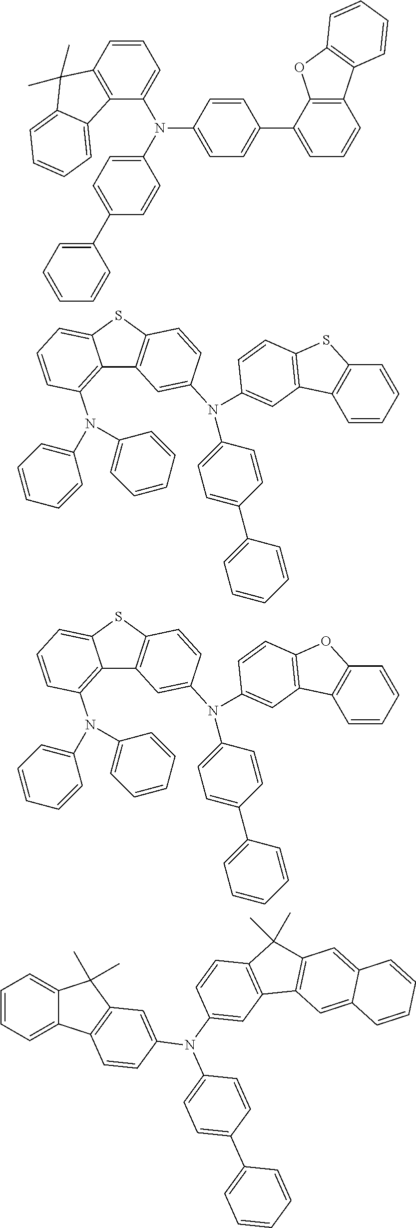

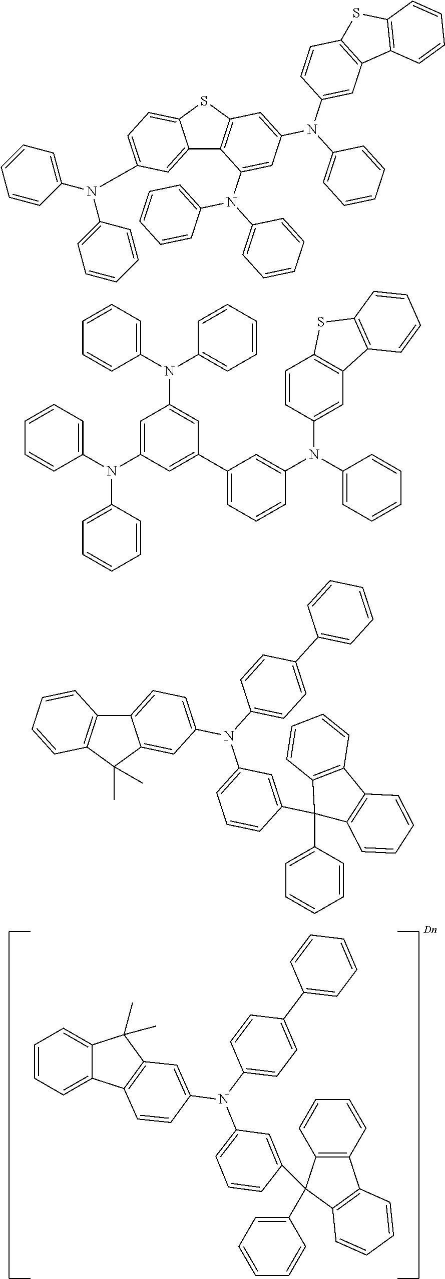

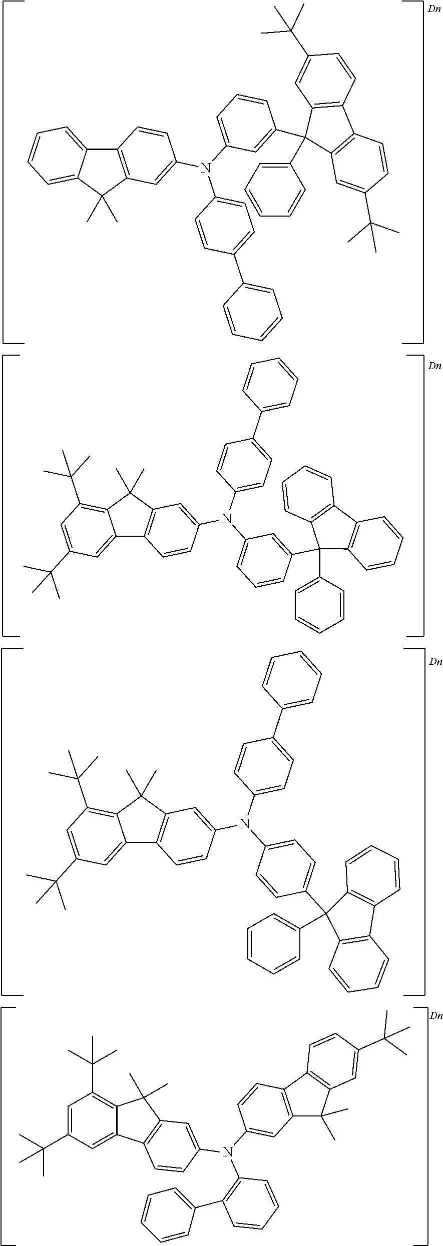

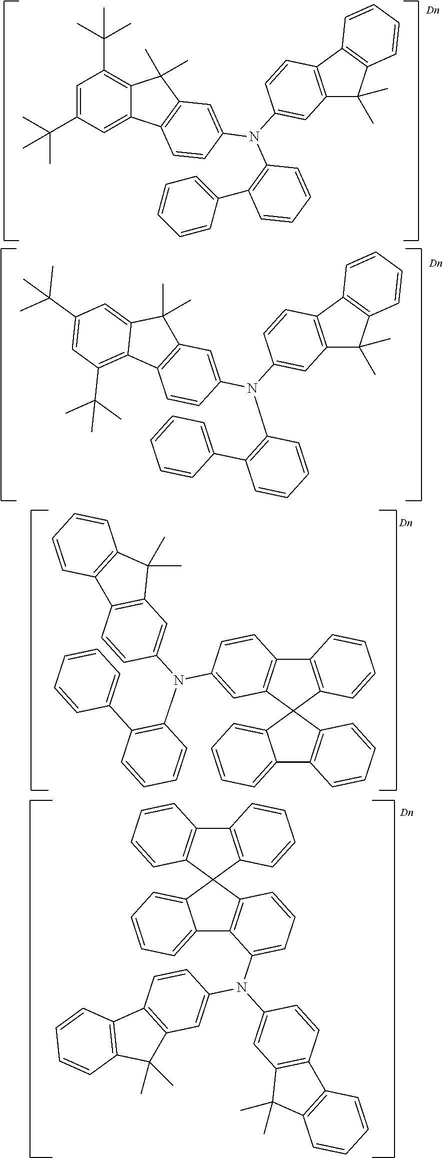

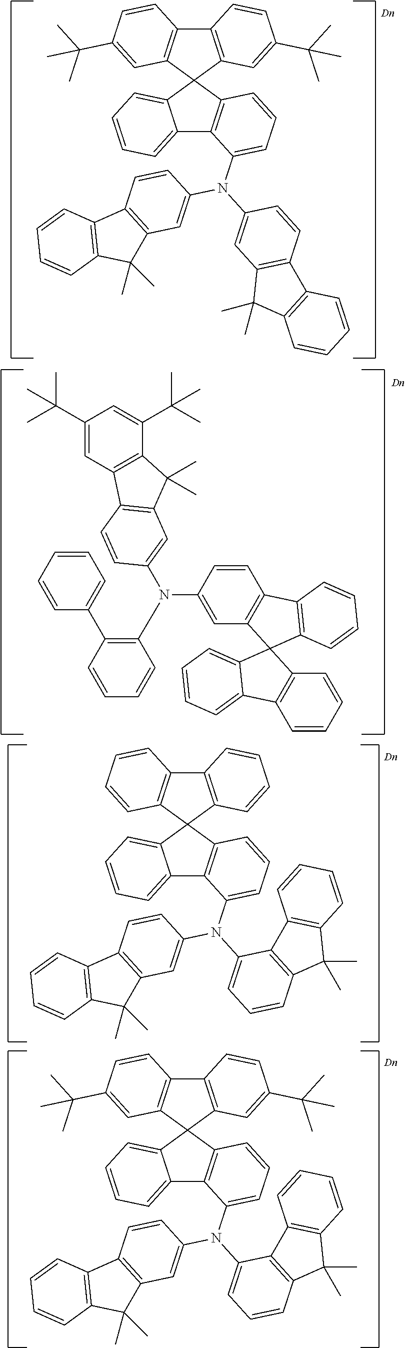

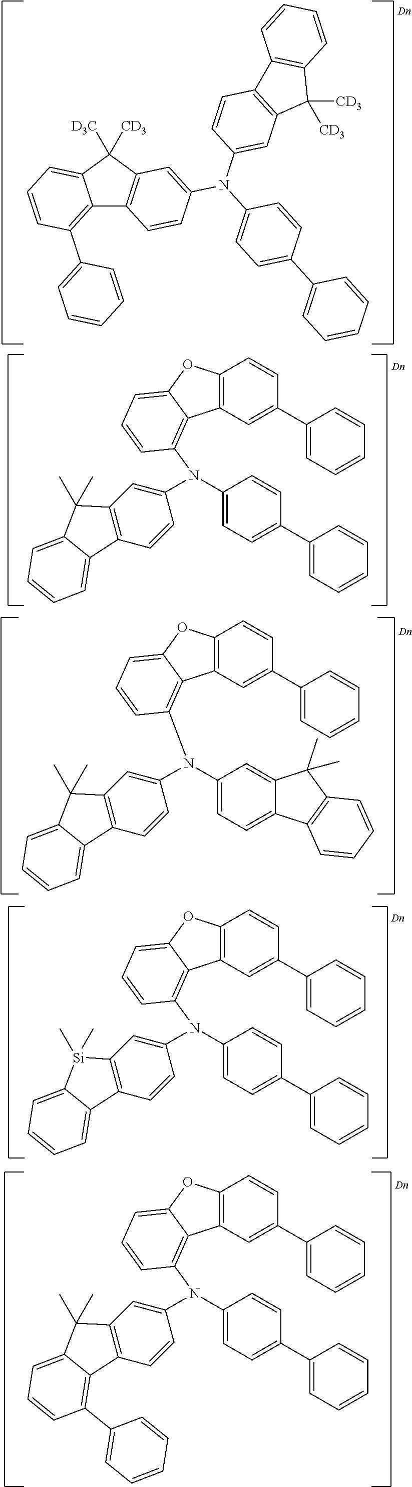

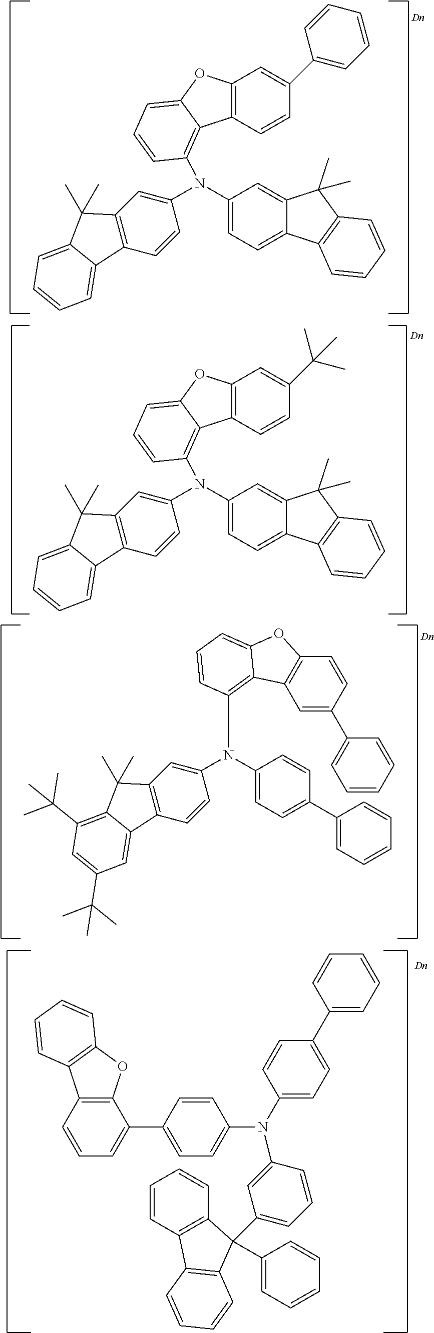

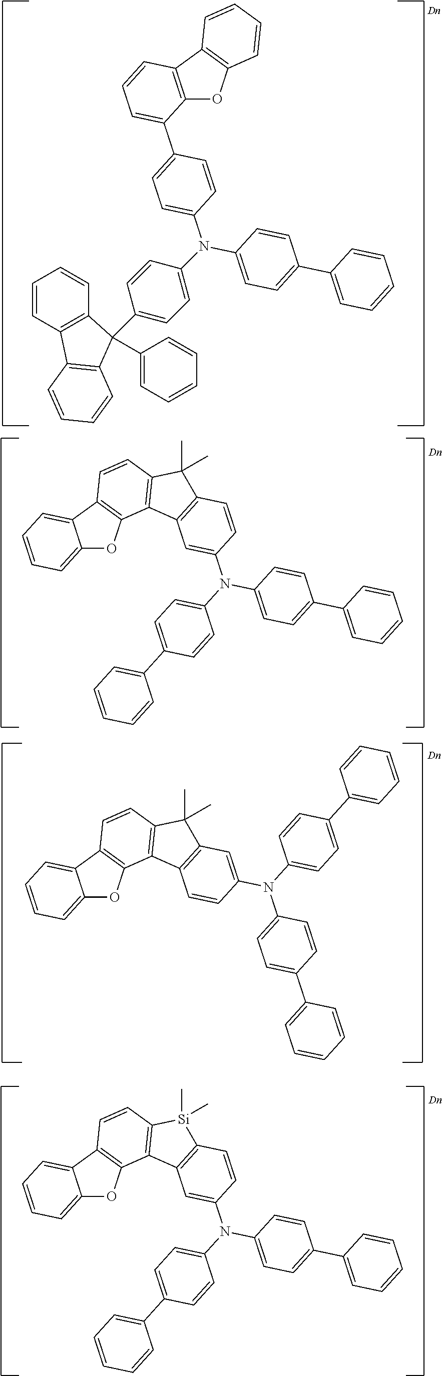

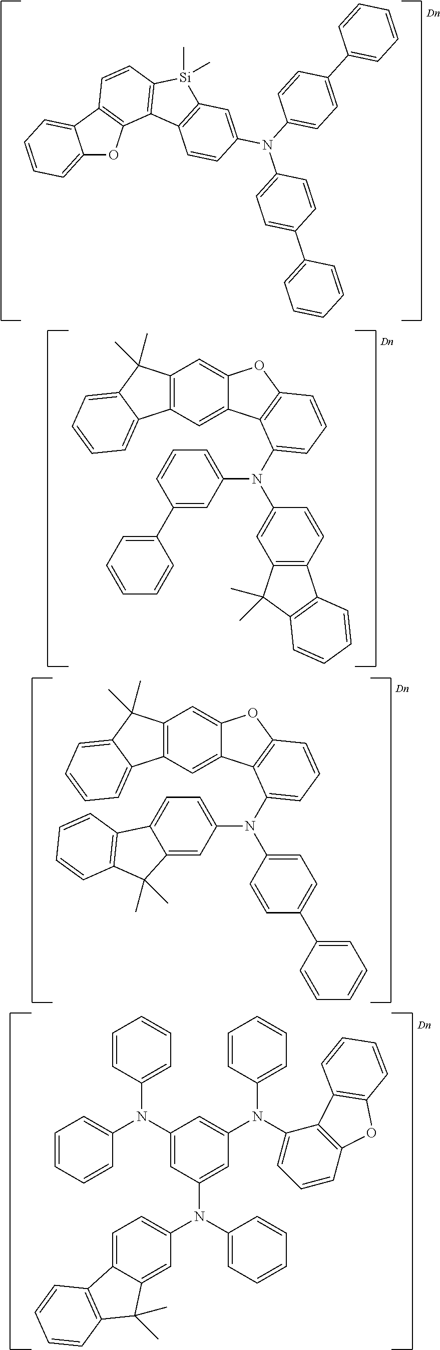

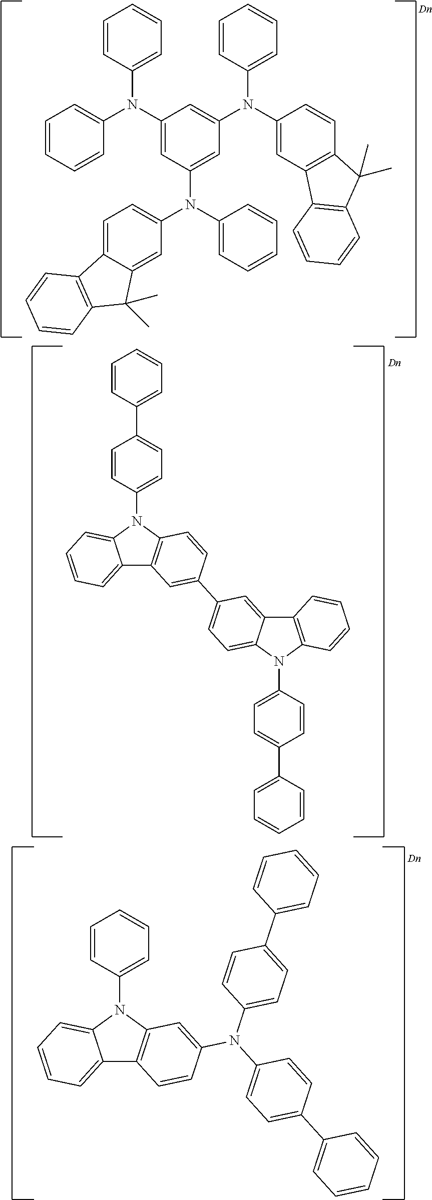

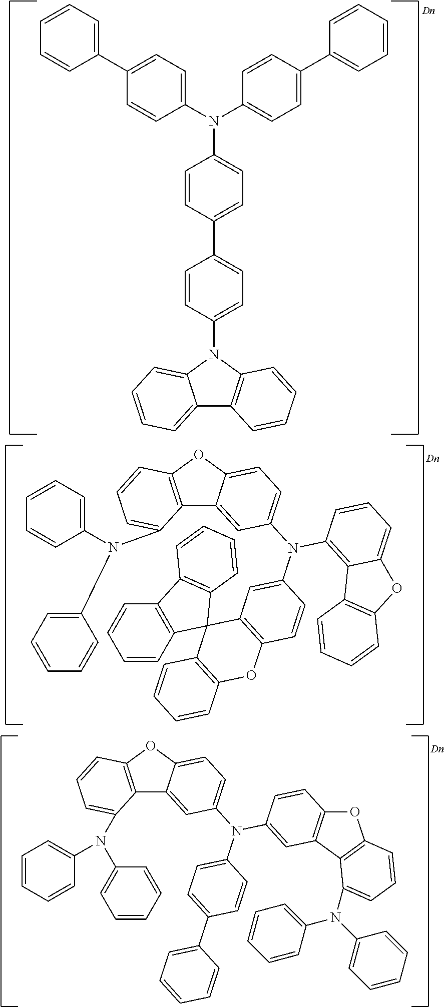

(Dn refers to the number of deuterium substitutions and indicates a structure substituted with one or more deuteriums)

In the hole transport region 140, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

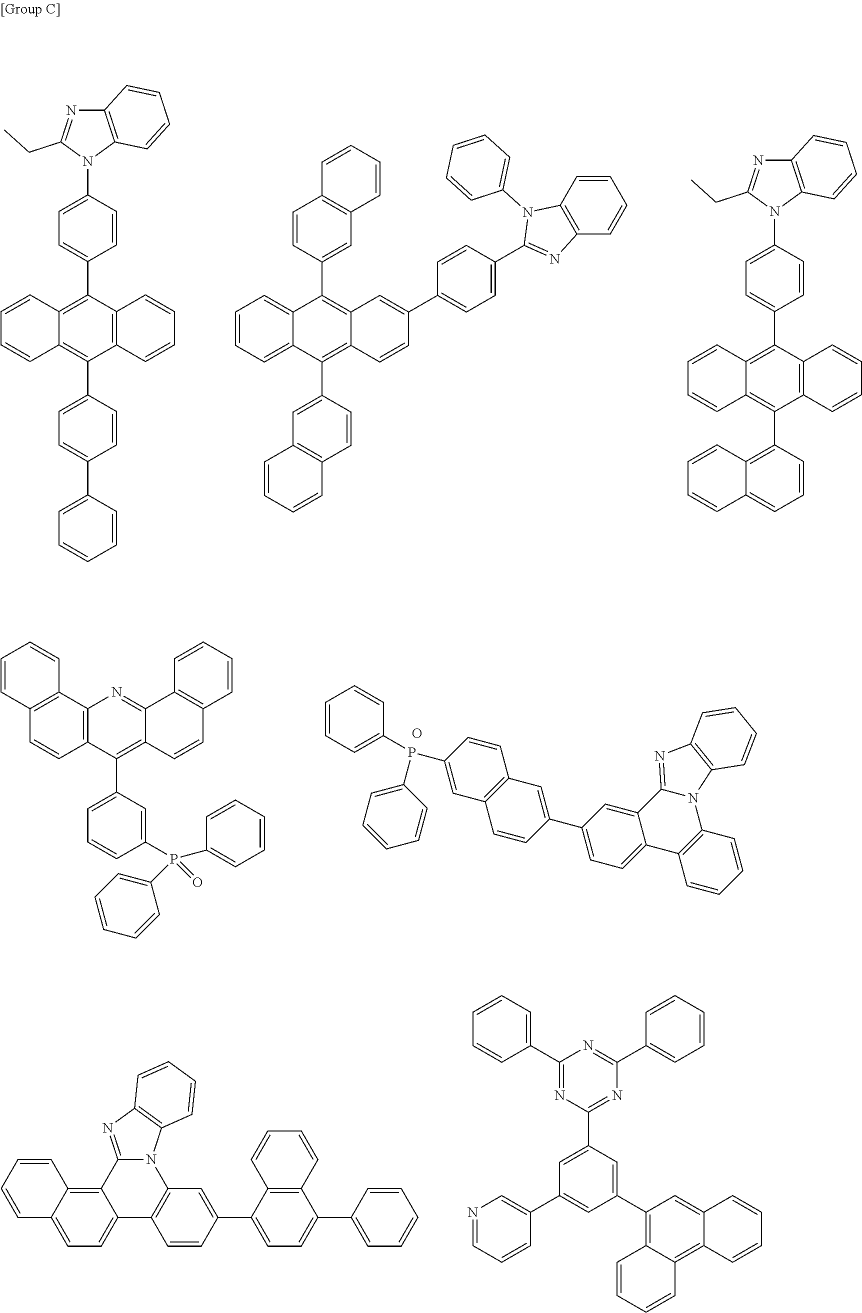

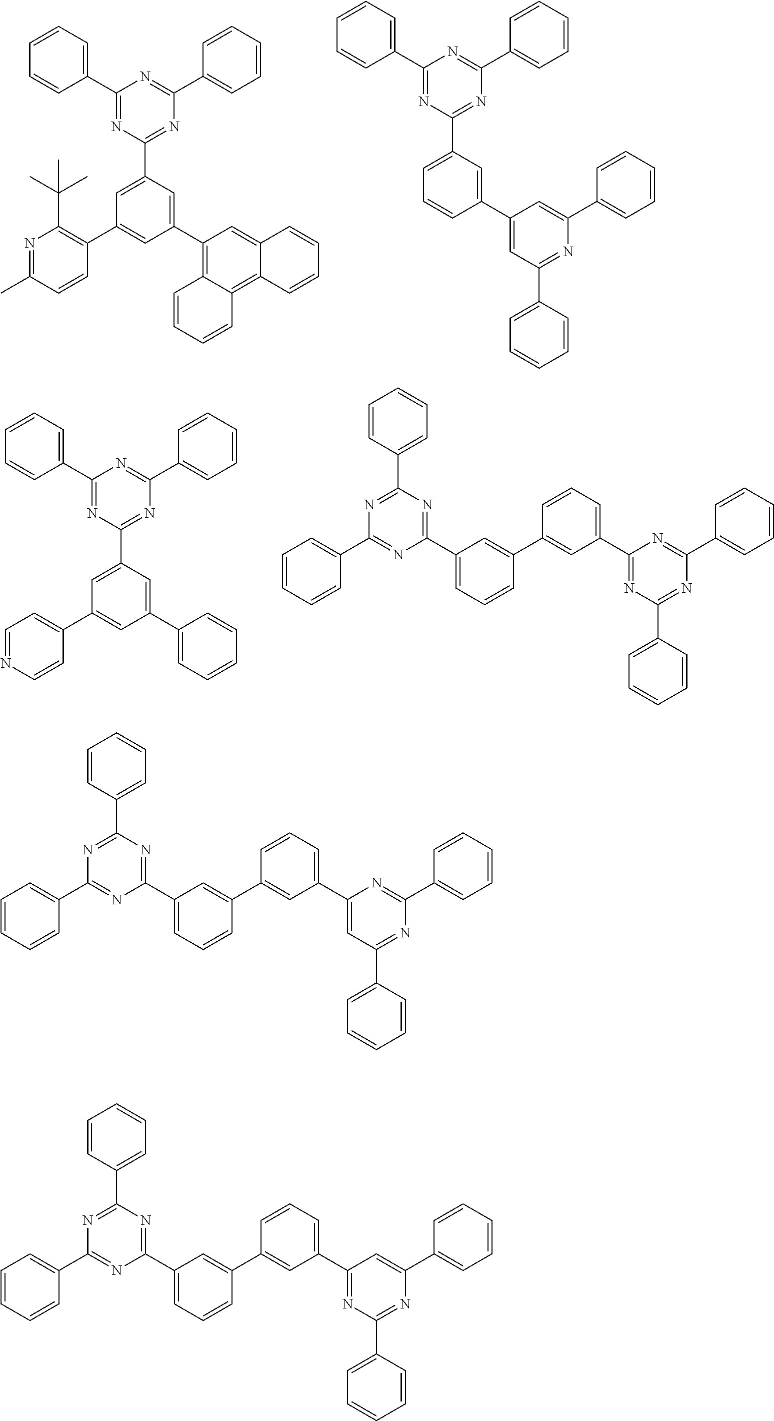 [SMILES: Cc1ccc(-c2cc(-c3nc(-c4ccccc4)nc(-c4ccccc4)n3)cc(-c3cc4ccccc4c4ccccc34)c2)c(C(C)(C)C)n1 ; c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

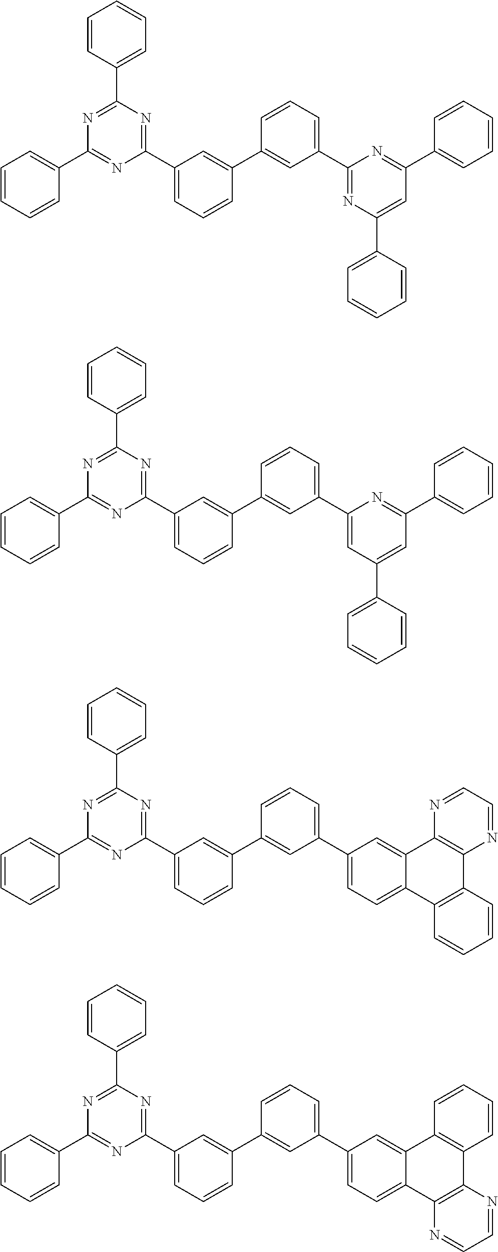 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

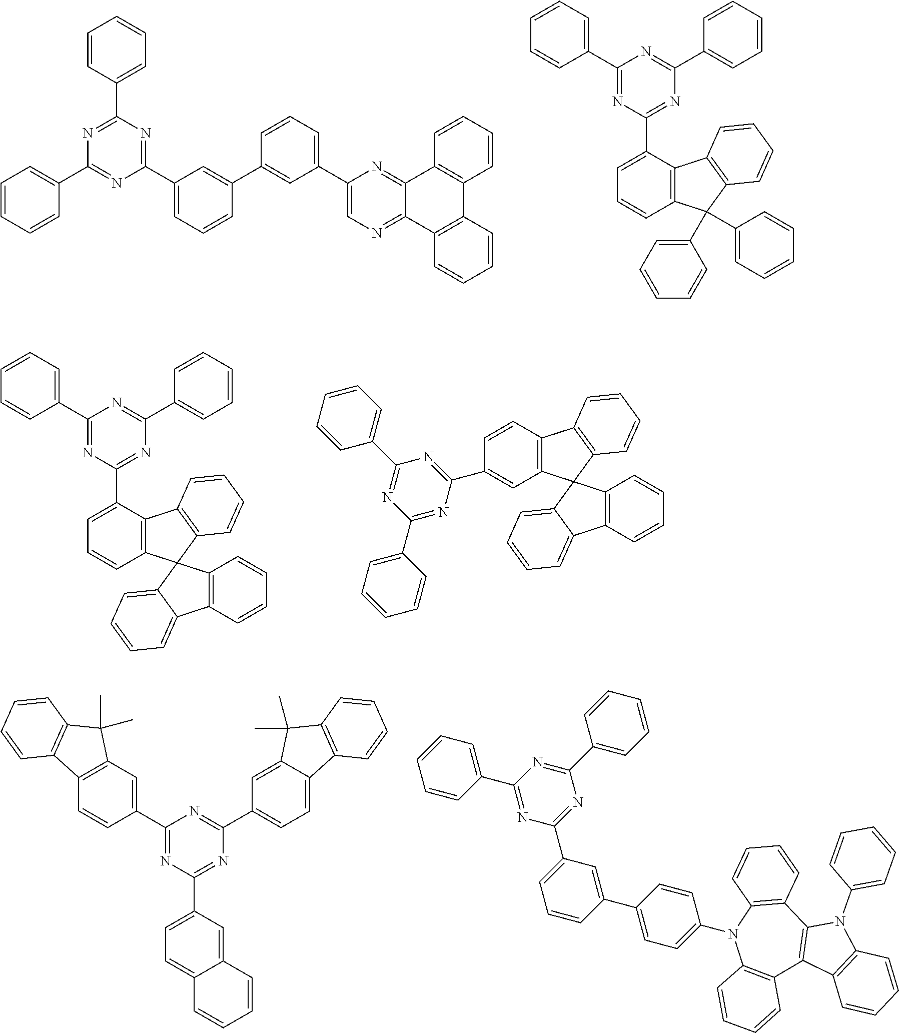

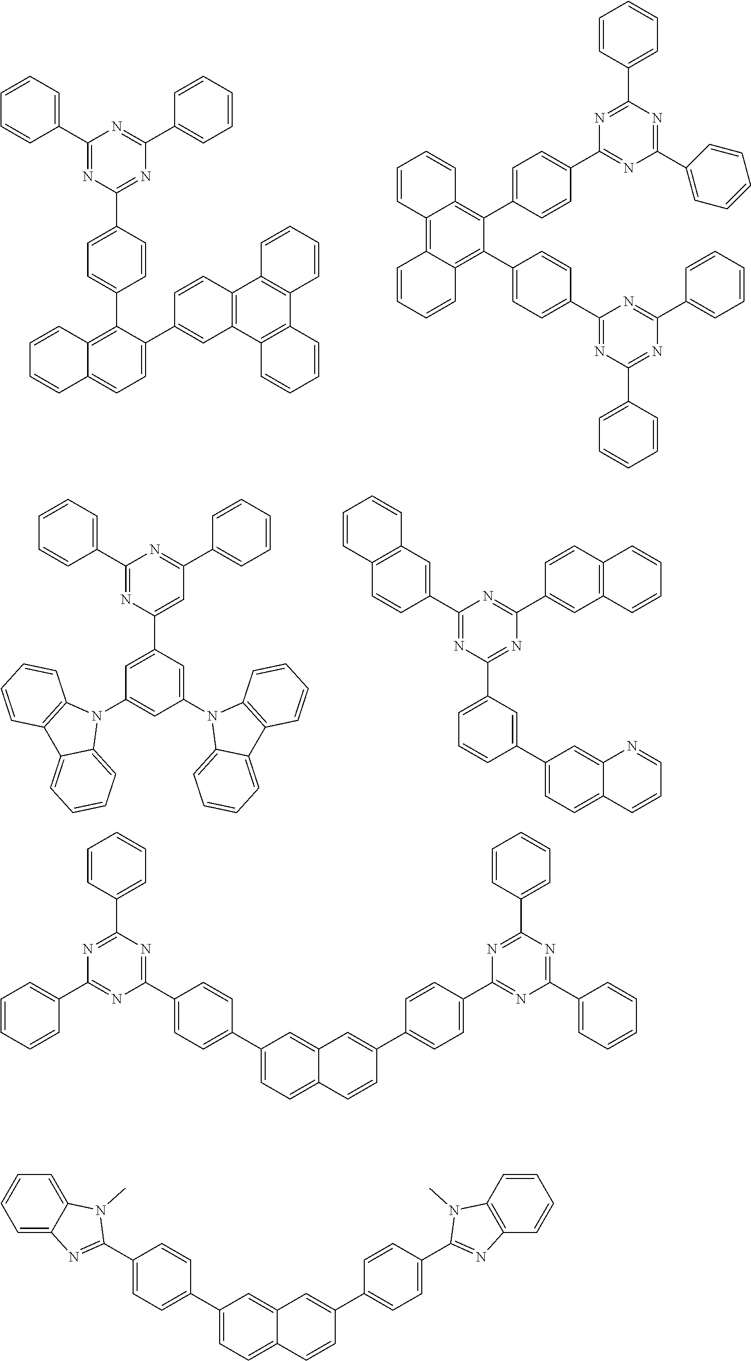

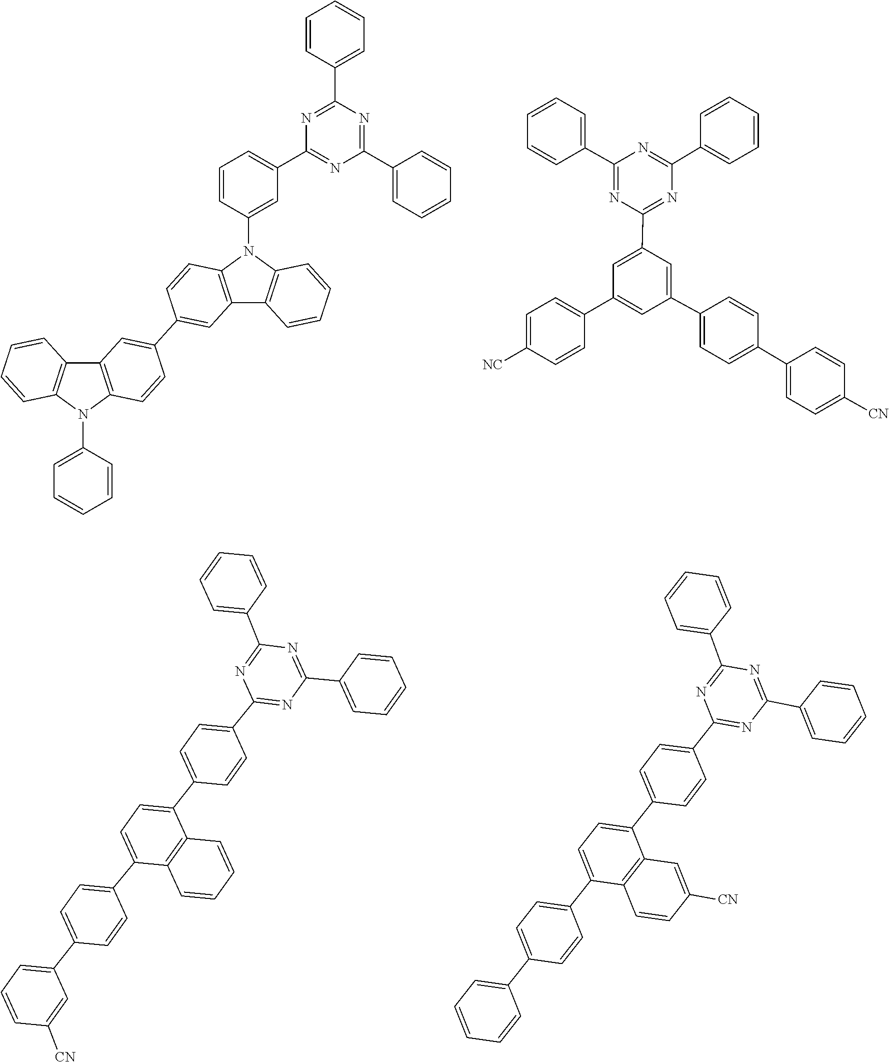 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

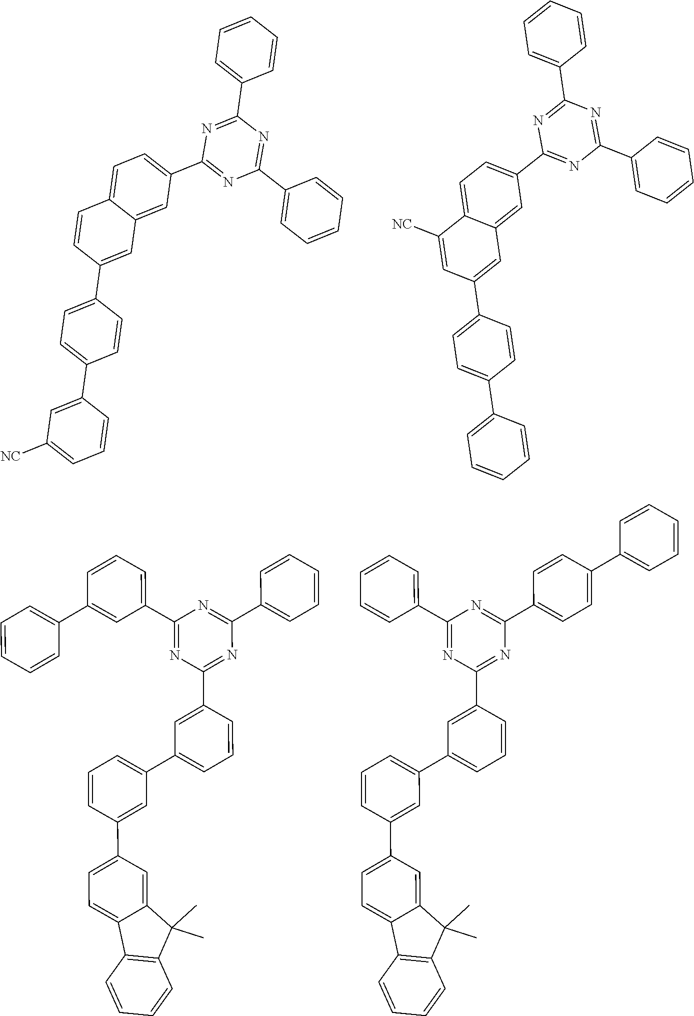

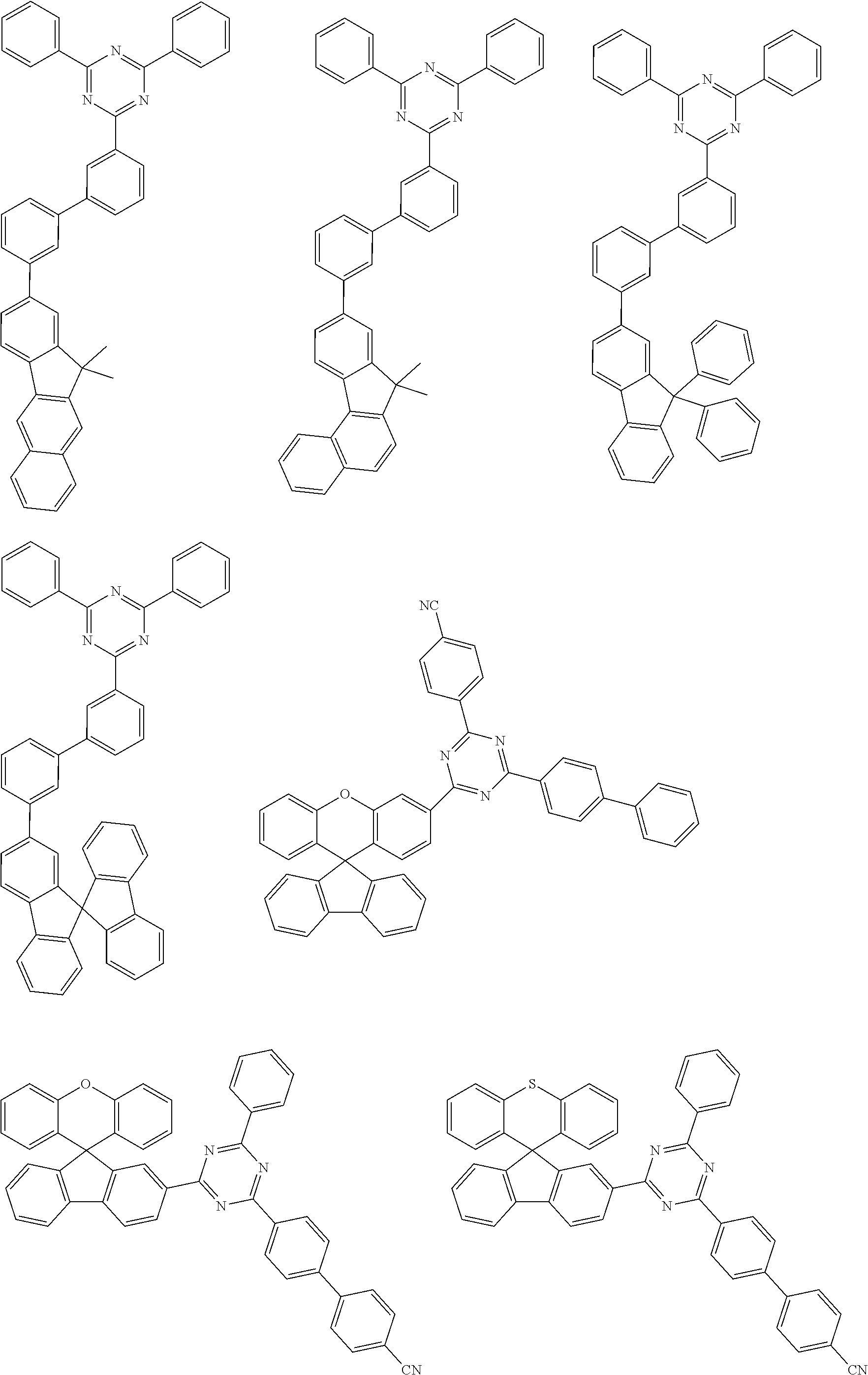

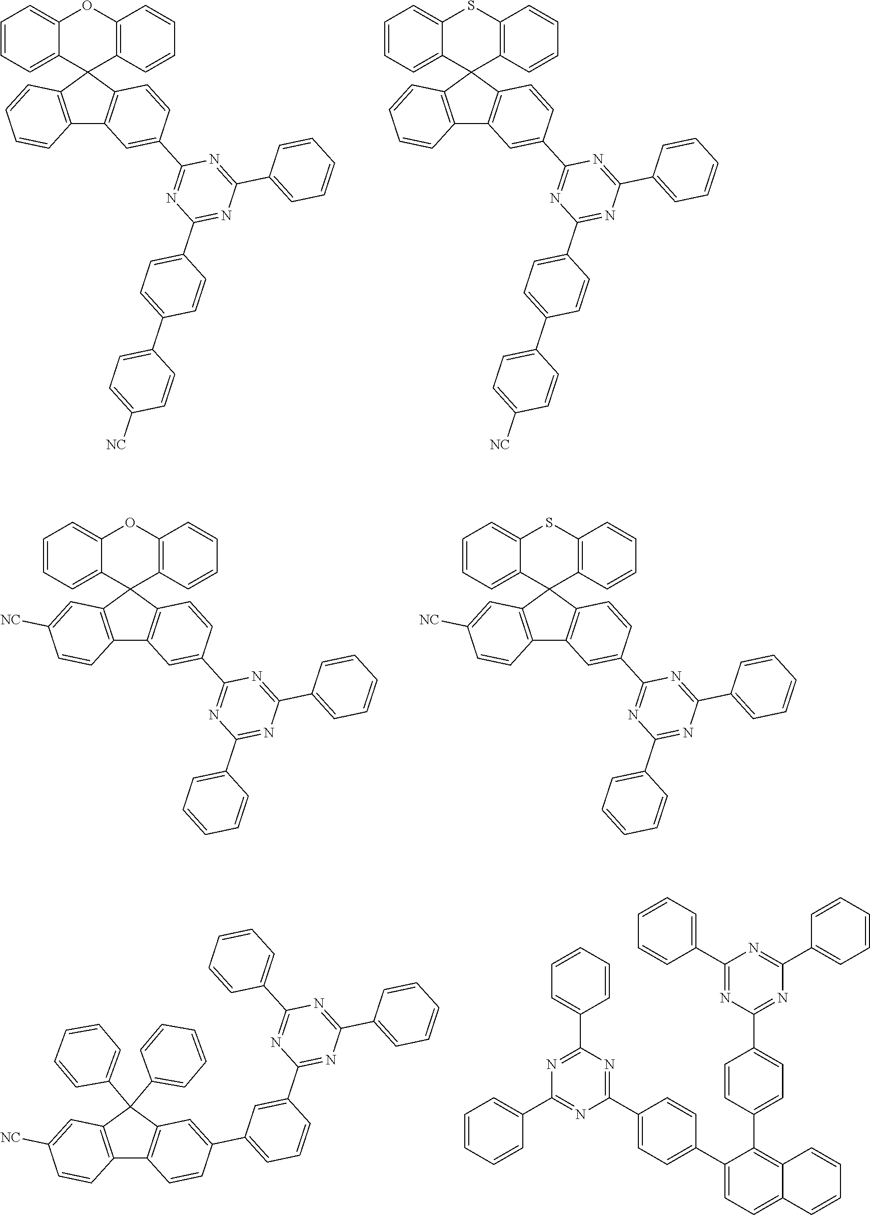

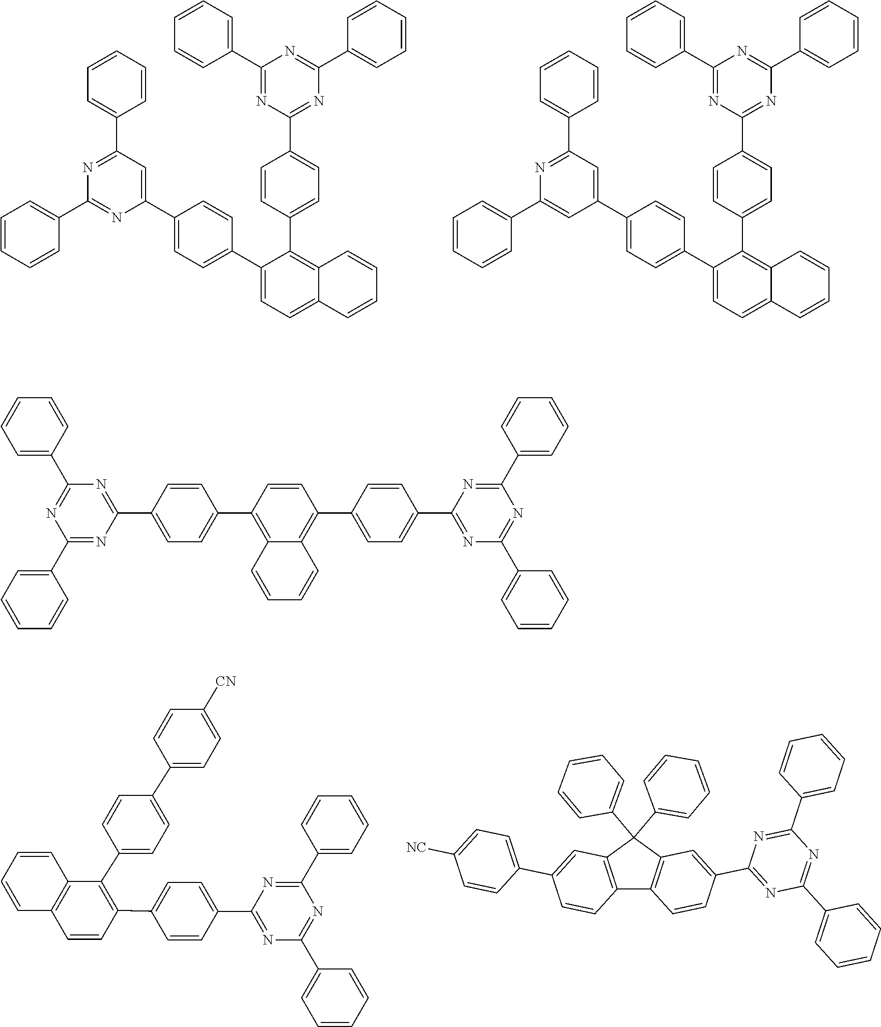 [SMILES: c1ccc(-c2cc(-c3ccc(-c4ccc5ccccc5c4-c4ccc(-c5nc(-c6ccccc6)nc(-c6ccccc6)n5)cc4)cc3)nc(-c3ccccc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)cc(-c3ccccc3)n2)cc1 ; c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1 ; N#Cc1ccc(-c2ccc(-c3c(-c4ccc(-c5nc(-c6ccccc6)nc(-c6ccccc6)n5)cc4)ccc4ccccc34)cc2)cc1 ; N#Cc1ccc(-c2ccc3c(c2)C(c2ccccc2)(c2ccccc2)c2cc(-c4nc(-c5ccccc5)nc(-c5ccccc5)n4)ccc2-3)cc1]

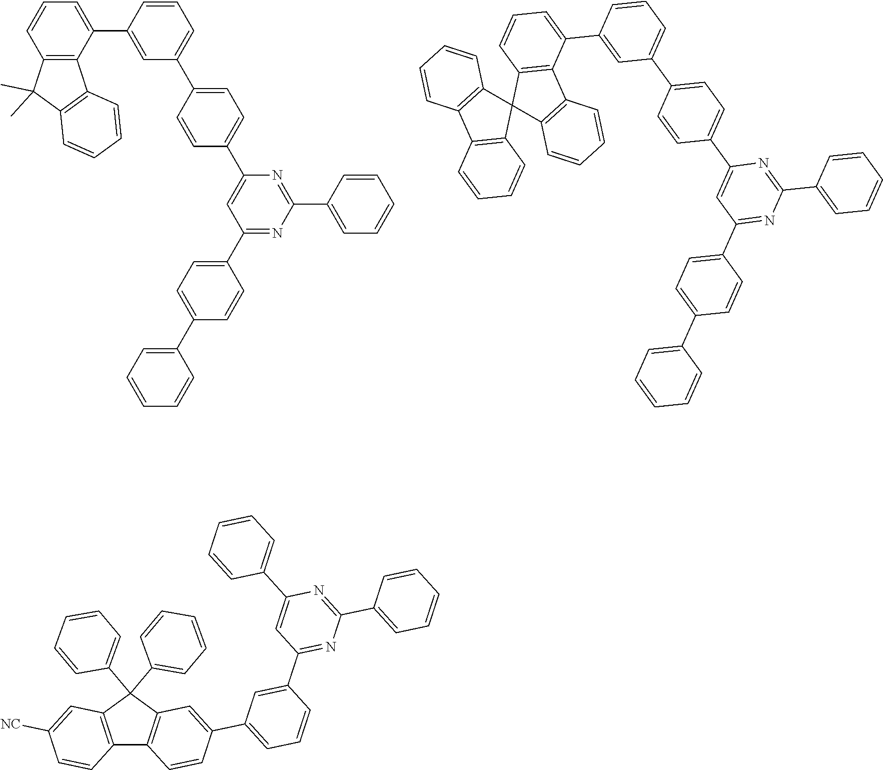 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4cc(-c5ccccc5)nc(-c5ccccc5)n4)c3)ccc1-2]

An embodiment may be an organic light emitting diode including the light emitting layer as the organic layer.

Another embodiment may be an organic light emitting diode including a light emitting layer and a hole transport region as the organic layer.

Another embodiment may be an organic light emitting diode including a light emitting layer and an electron transport region as the organic layer.

Another embodiment of the present invention may provide an organic light emitting diode including a hole transport region 140 and an electron transport region 150 in addition to the light emitting layer 130 as the organic layer 105, as shown in FIG. 1.

On the other hand, an organic light emitting diode may further include an electron injection layer (not shown), a hole injection layer (not shown), etc. in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The aforementioned organic light emitting diode may be applied to an organic light emitting display device.

### MODE FOR INVENTION

Hereinafter, the embodiments are illustrated in more detail with reference to examples. However, these examples are exemplary, and the scope of claims is not limited thereto.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by known methods.

### Preparation of Compound for Organic Optoelectronic Device

### Synthesis Example 1: Synthesis of Compound A-12

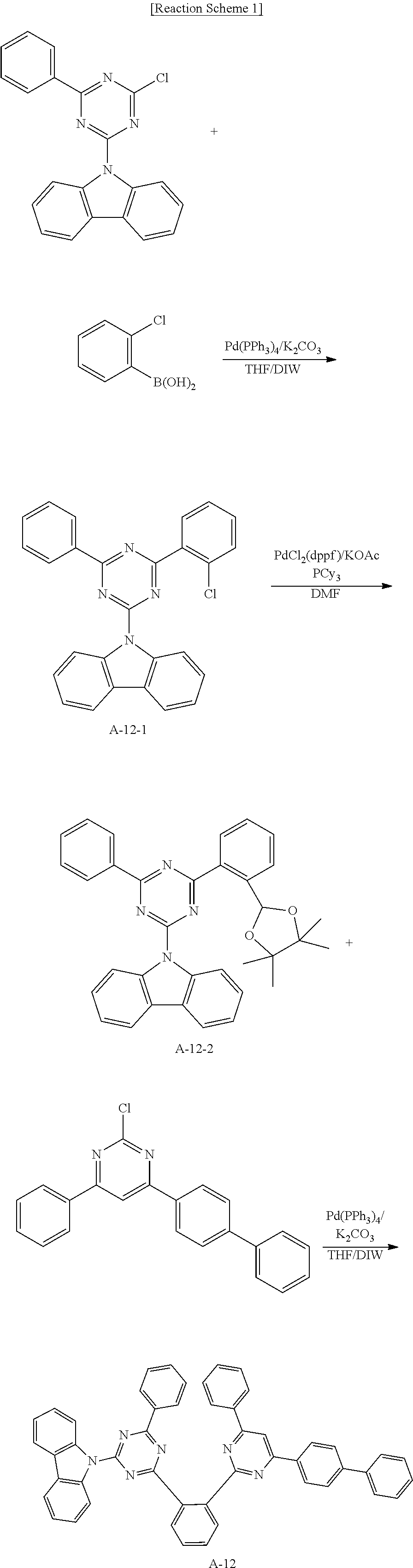

### 1st Step: Synthesis of Intermediate A-12-1

9-(4-chloro-6-phenyl-1,3,5-triazin-2-yl)-9H-carbazole (50.0 g, 140.1 mmol), 2-chlorophenylboronic acid (23.0 g, 147.1 mmol), tetrakis(triphenylph osphine)palladium (0)(8.1 g, 7.0 mmol), and potassium carbonate (58.1 g, 420.4 mmol) were dissolved in 750 mL of a mixed solution of tetrahydrofuran:distilled water=2:1 in a volume ratio and then, stirred under reflux at 80° C. for 12 hours. When a reaction was completed, purification by column chromatography (dichloromethane: n-hexane) was performed, obtaining 51.4 g (Yield: 84.7%) of Intermediate A-12-1.

### 2nd Step: Synthesis of Intermediate A-12-2

Intermediate A-12-1 (51.4 g, 118.7 mmol), bis(pinacolato)diboron (39.2 g, 154.4 mmol), [1,1′-bis(diphenylphosphino)ferrocene]palladium(II) dichloride (4.8 g, 5.9 mmol), potassium acetate (35.0 g, 356.2 mmol), and tricyclohexylphosphine (6.7 g, 23.8 mmol) were dissolved in 600 mL of N,N-dimethylformamide and then, stirred under reflux at 140° C. for 12 hours. When a reaction was completed, the resultant was poured into an excessive amount of distilled water to completely precipitate solids. The precipitated solids were filtered and dissolved by boiling in toluene and then, filtered again through silica gel. The filtered solution was recrystallized to obtain 27.1 g (Yield: 43.6%) of Intermediate A-12-2.

### 3rd Step: Synthesis of Compound A-12

Intermediate A-12-2 and 4-([1,1′-biphenyl]-4-yl)-2-chloro-6-phenylpyrimidine were used as starting materials to synthesize Intermediate A-12-1 of Synthesis Example 1 in the same method. Purification by recrystallization with toluene was performed to obtain 4.7 g (Yield: 69.2%) of Compound A-12.

### Synthesis Examples 2 to 9

Reactant 1 and Reactant 2 were used as starting materials through the 3^{rd} step reaction (Suzuki rxn) of Synthesis Example 1 to synthesize compounds shown in Table 1.

TABLE 1

| Synthesis | | | | |
|---|---|---|---|---|
| Example | Reactant 1 | Reactant 2 | Product | Yield |
| Synthesis Example 2 | 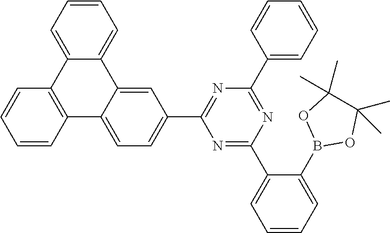 [SMILES: CC1(C)OB(c2ccccc2-c2nc(-c3ccccc3)nc(-c3ccc4c5ccccc5c5ccccc5c4c3)n2)OC1(C)C] | 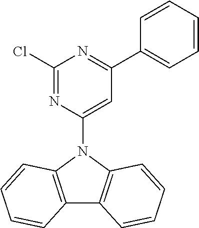 [SMILES: Clc1nc(-c2ccccc2)cc(-n2c3ccccc3c3ccccc32)n1] | Compound A-20 | 81.3% |
| Synthesis Example 3 | 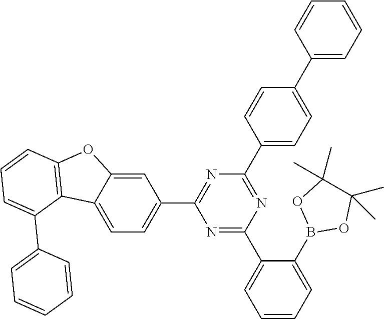 [SMILES: CC1(C)OB(c2ccccc2-c2nc(-c3ccc(-c4ccccc4)cc3)nc(-c3ccc4c(c3)oc3cccc(-c5ccccc5)c34)n2)OC1(C)C] | 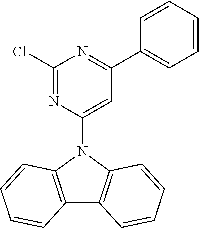 [SMILES: Clc1nc(-c2ccccc2)cc(-n2c3ccccc3c3ccccc32)n1] | Compound A-89 | 66.4% |
| Synthesis Example 4 | 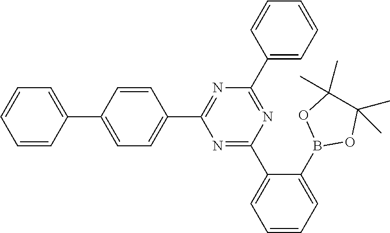 [SMILES: CC1(C)OB(c2ccccc2-c2nc(-c3ccccc3)nc(-c3ccc(-c4ccccc4)cc3)n2)OC1(C)C] | 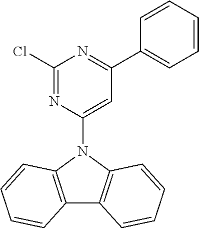 [SMILES: Clc1nc(-c2ccccc2)cc(-n2c3ccccc3c3ccccc32)n1] | Compound A-24 | 59.2% |
| Synthesis Example 5 | 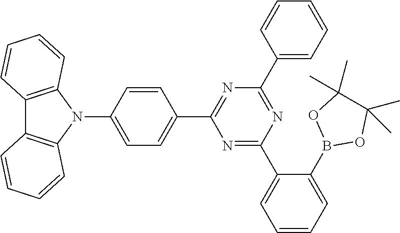 [SMILES: CC1(C)OB(c2ccccc2-c2nc(-c3ccccc3)nc(-c3ccc(-n4c5ccccc5c5ccccc54)cc3)n2)OC1(C)C] | 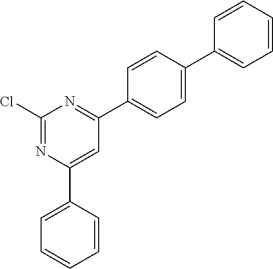 [SMILES: Clc1nc(-c2ccccc2)cc(-c2ccc(-c3ccccc3)cc2)n1] | Compound A-51 | 69.9% |
| Synthesis Example 6 | 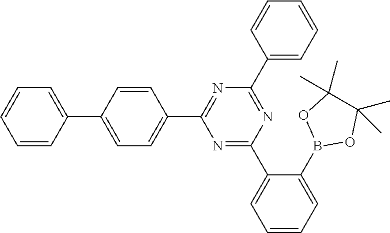 [SMILES: CC1(C)OB(c2ccccc2-c2nc(-c3ccccc3)nc(-c3ccc(-c4ccccc4)cc3)n2)OC1(C)C] | 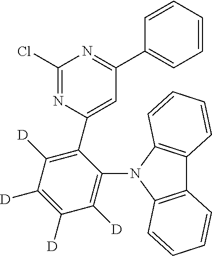 [SMILES: [2H]c1c([2H])c([2H])c(-n2c3ccccc3c3ccccc32)c(-c2cc(-c3ccccc3)nc(Cl)n2)c1[2H]] | Compound A-57 | 72.3% |
| Synthesis Example 7 | 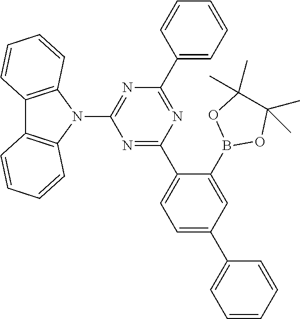 [SMILES: CC1(C)OB(c2cc(-c3ccccc3)ccc2-c2nc(-c3ccccc3)nc(-n3c4ccccc4c4ccccc43)n2)OC1(C)C] | 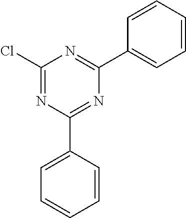 [SMILES: Clc1nc(-c2ccccc2)nc(-c2ccccc2)n1] | Compound A-74 | 49.5% |
| Synthesis Example 8 | 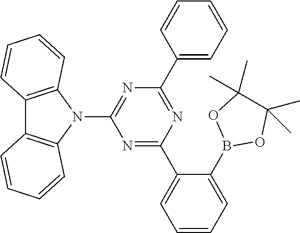 [SMILES: CC1(C)OB(c2ccccc2-c2nc(-c3ccccc3)nc(-n3c4ccccc4c4ccccc43)n2)OC1(C)C] | 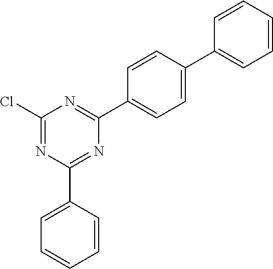 [SMILES: Clc1nc(-c2ccccc2)nc(-c2ccc(-c3ccccc3)cc2)n1] | Compound A-76 | 62.3% |
| Synthesis Example 9 | 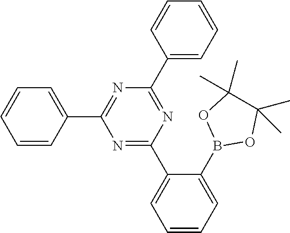 [SMILES: CC1(C)OB(c2ccccc2-c2nc(-c3ccccc3)nc(-c3ccccc3)n2)OC1(C)C] | 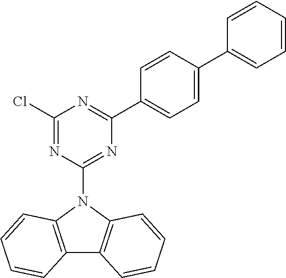 [SMILES: Clc1nc(-c2ccc(-c3ccccc3)cc2)nc(-n2c3ccccc3c3ccccc32)n1] | Compound A-84 | 77.0% |

### Comparative Synthesis Examples 1 to 6

Reactant 1 and Reactant 2 were used as starting materials through the 3^{rd} step reaction (Suzuki rxn) of Synthesis Example 1 to synthesize compounds shown in Table 2.

TABLE 2

| Synthesis | | |
|---|---|---|
| Example | Reactant 1 | Reactant 2 |
| Comparative Synthesis Example 1 | 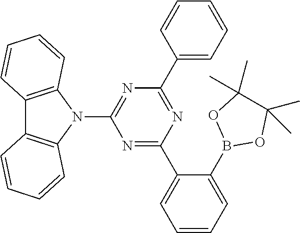 [SMILES: CC1(C)OB(c2ccccc2-c2nc(-c3ccccc3)nc(-n3c4ccccc4c4ccccc43)n2)OC1(C)C] | 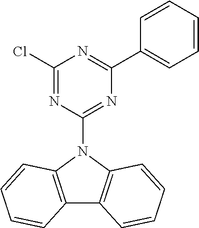 [SMILES: Clc1nc(-c2ccccc2)nc(-n2c3ccccc3c3ccccc32)n1] |
| Comparative Synthesis Example 2 | 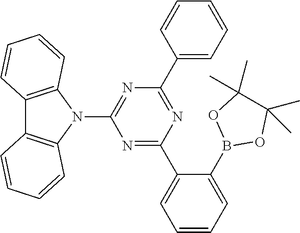 [SMILES: CC1(C)OB(c2ccccc2-c2nc(-c3ccccc3)nc(-n3c4ccccc4c4ccccc43)n2)OC1(C)C] | 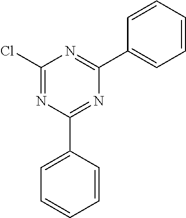 [SMILES: Clc1nc(-c2ccccc2)nc(-c2ccccc2)n1] |
| Comparative Synthesis Example 3 | 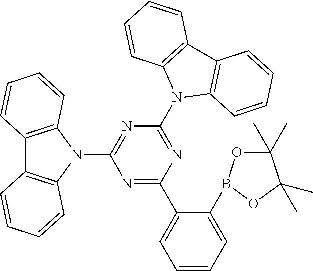 [SMILES: CC1(C)OB(c2ccccc2-c2nc(-n3c4ccccc4c4ccccc43)nc(-n3c4ccccc4c4ccccc43)n2)OC1(C)C] | 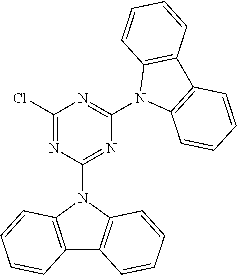 [SMILES: Clc1nc(-n2c3ccccc3c3ccccc32)nc(-n2c3ccccc3c3ccccc32)n1] |
| Comparative Synthesis Example 4 | 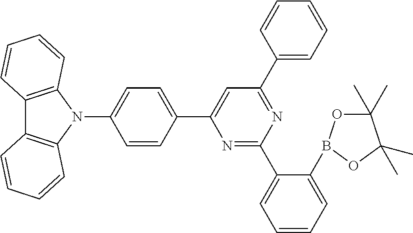 [SMILES: CC1(C)OB(c2ccccc2-c2nc(-c3ccccc3)cc(-c3ccc(-n4c5ccccc5c5ccccc54)cc3)n2)OC1(C)C] | 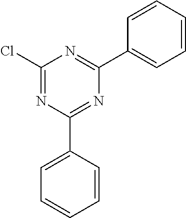 [SMILES: Clc1nc(-c2ccccc2)nc(-c2ccccc2)n1] |
| Comparative Synthesis Example 5 | 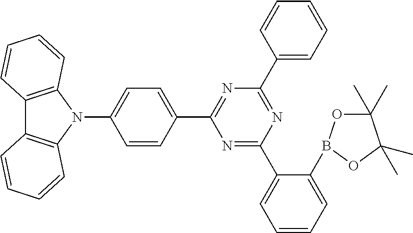 [SMILES: CC1(C)OB(c2ccccc2-c2nc(-c3ccccc3)nc(-c3ccc(-n4c5ccccc5c5ccccc54)cc3)n2)OC1(C)C] | 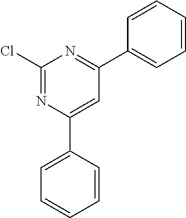 [SMILES: Clc1nc(-c2ccccc2)cc(-c2ccccc2)n1] |
| Comparative Synthesis Example 6 | 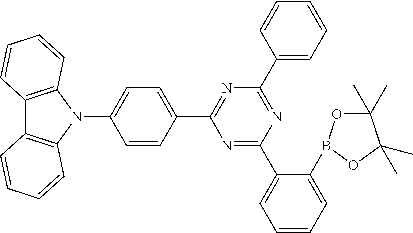 [SMILES: CC1(C)OB(c2ccccc2-c2nc(-c3ccccc3)nc(-c3ccc(-n4c5ccccc5c5ccccc54)cc3)n2)OC1(C)C] | 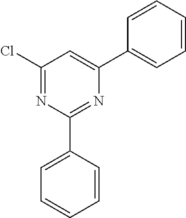 [SMILES: Clc1cc(-c2ccccc2)nc(-c2ccccc2)n1] |

| Synthesis | | |
|---|---|---|
| Example | Product | Yield |
| Comparative Synthesis Example 1 | 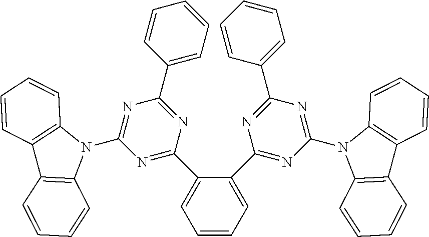 [SMILES: c1ccc(-c2nc(-c3ccccc3-c3nc(-c4ccccc4)nc(-n4c5ccccc5c5ccccc54)n3)nc(-n3c4ccccc4c4ccccc43)n2)cc1] | 68.3% |
| | Compound C1 | |
| Comparative Synthesis Example 2 | 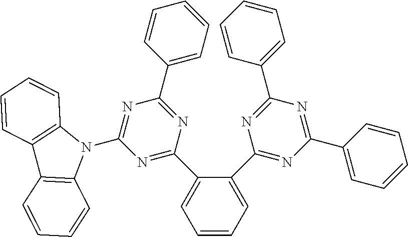 [SMILES: c1ccc(-c2nc(-c3ccccc3)nc(-c3ccccc3-c3nc(-c4ccccc4)nc(-n4c5ccccc5c5ccccc54)n3)n2)cc1] | 77.2% |
| | Compound C2 | |
| Comparative Synthesis Example 3 | 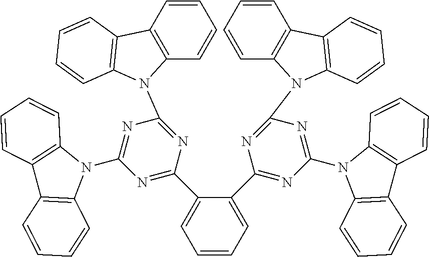 [SMILES: c1ccc(-c2nc(-n3c4ccccc4c4ccccc43)nc(-n3c4ccccc4c4ccccc43)n2)c(-c2nc(-n3c4ccccc4c4ccccc43)nc(-n3c4ccccc4c4ccccc43)n2)c1] | 35.2% |
| | Compound C3 | |
| Comparative Synthesis Example 4 | 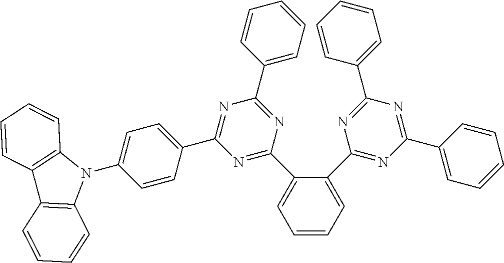 [SMILES: c1ccc(-c2nc(-c3ccccc3)nc(-c3ccccc3-c3nc(-c4ccccc4)nc(-c4ccc(-n5c6ccccc6c6ccccc65)cc4)n3)n2)cc1] | 62.2% |
| | Compound 4 | |
| Comparative Synthesis Example 5 | 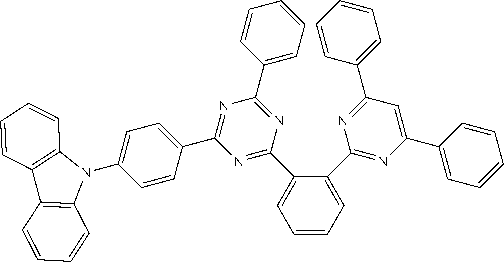 [SMILES: c1ccc(-c2cc(-c3ccccc3)nc(-c3ccccc3-c3nc(-c4ccccc4)nc(-c4ccc(-n5c6ccccc6c6ccccc65)cc4)n3)n2)cc1] | 75.9% |
| | Compound C5 | |
| Comparative Synthesis Example 6 | 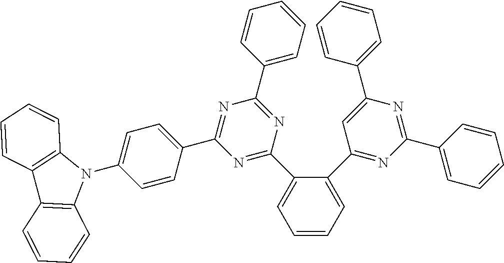 [SMILES: c1ccc(-c2cc(-c3ccccc3-c3nc(-c4ccccc4)nc(-c4ccc(-n5c6ccccc6c6ccccc65)cc4)n3)nc(-c3ccccc3)n2)cc1] | 74.3% |
| | Compound C6 | |

### Preparation of Second Compound

### Synthesis Example 10: Synthesis of Compound B-239

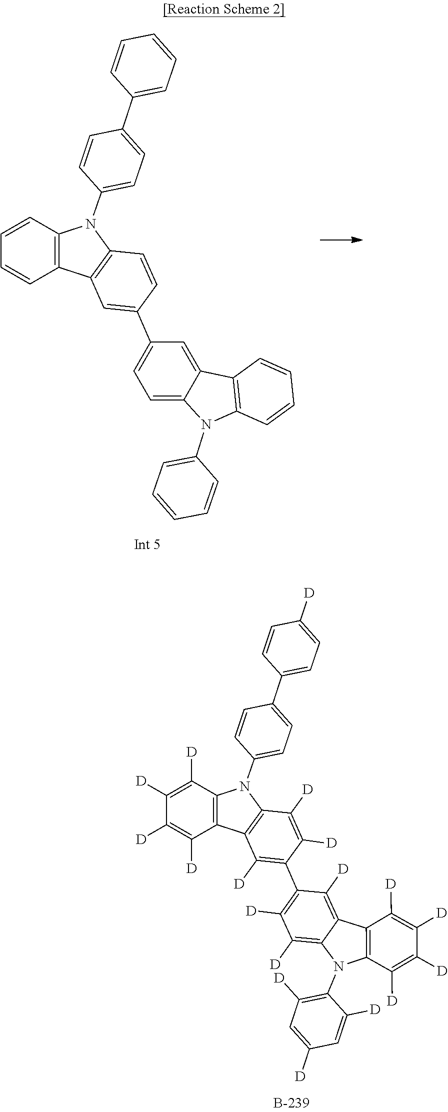 [SMILES: c1ccc(-c2ccc(-n3c4ccccc4c4cc(-c5ccc6c(c5)c5ccccc5n6-c5ccccc5)ccc43)cc2)cc1 ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)cc1]

### 1st Step: Synthesis of Compound Int 5

Compound Int 5 was synthesized by referring to the method disclosed in Korean Publication No. 2016-0049842.

### 2nd Step: Synthesis of Compound B-239

30 g (0.0535 mol) of Compound Int 5, 40 g (0.267 mol) of trifluoromethanesulfonic acid, and 282 g (3.35 mol) of D₆-benzene were added and then, stirred at 10° C. for 24 hours. Purified water was added thereto and then, neutralized by using a saturated K₃PO₄ solution. An organic layer therefrom was concentrated and column-purified to obtain 18 g of Compound B-239 (a white solid, LC-Mass Mz 578.79, C42H₁₀D₁₈N₂).

### (Manufacture of Organic Light Emitting Diode)

### Example 1

A glass substrate coated with a thin film of ITO (indium tin oxide) was ultrasonically cleaned with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, and Compound A doped with 3% NDP-9 (commercially available from Novaled) was vacuum-deposited on the ITO substrate to form a 100 Å-thick hole injection layer, and a 1350 Å-thick hole transport layer was formed thereon by depositing Compound A. Compound B was deposited on the hole transport layer to a thickness of 350 Å to form a hole transport auxiliary layer. Compound A-74 obtained in Synthesis Example 7 was used as a host on the hole transport auxiliary layer and PhGD was doped as a dopant at 7 wt % to form a 400 Å-thick light emitting layer by vacuum deposition. Subsequently, Compound C was deposited on the light emitting layer to a thickness of 50 Å to form an electron transport auxiliary layer, and Compound D and LiQ were simultaneously vacuum-deposited at a weight ratio of 1:1 to form a 300 Å-thick electron transport layer. An organic light emitting diode was manufactured by sequentially vacuum-depositing 15 Å of LiQ and 1200 Å of Al on the electron transport layer to form a cathode.

The organic light emitting diode was manufactured to have a structure of ITO/Compound A (3% NDP-9 doping, 100 Å)/Compound A (1350 Å)/Compound B (350 Å)/EML [93 wt % of host (Compound A-74): 7 wt % of PhGD](400 Å)/Compound C (50 Å)/Compound D: LiQ (300 Å)/LiQ (15 Å)/Al (1200 Å).

Compound A: N-(biphenyl-4-yl)-9,9-dimethyl-N-(4-(9-phenyl-9H-carbazol-3-yl)phenyl)-9H-fluoren-2-amine

Compound B: N-[4-(4-dibenzofuranyl)phenyl]-N-[4-(9-phenyl-9H-fluoren-9-yl)phenyl][1,1′-biphenyl]-4-amine

Compound C: 2,4-diphenyl-6-(4′,5′,6′-triphenyl[1,1′:2′,1″:3″,1′″:3′″,1″″-quinquephenyl]-3″″-yl)-1,3,5-triazine

Compound D: 2-(1,1′-biphenyl-4-yl)-4-(9,9-diphenylfluoren-4-yl)-6-phenyl-1,3,5-triazine

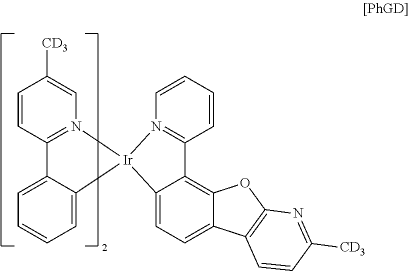

### Examples 2 to 9 and Comparative Examples 1 to 6

Diodes of Examples 2 to 9 and Comparative Examples 1 to 6 were manufactured in the same manner as in Example 1, except that the host was changed as described in Tables 3 and 4.

### Example 10

A glass substrate coated with a thin film of ITO (indium tin oxide) was ultrasonically cleaned with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to form a 1350 Å-thick hole transport layer. Compound E was deposited on the hole transport layer to a thickness of 350 Å to form a hole transport auxiliary layer. Compound A-12 obtained in Synthesis Example 1 and Compound B-239 synthesized in Synthesis Example 10 were simultaneously used as hosts on the hole transport auxiliary layer, and PhGD was doped at 10 wt % as a dopant to form a 330 Å-thick light emitting layer by vacuum deposition. Subsequently, Compound F was deposited on the light emitting layer to form a 50 Å-thick electron transport auxiliary layer, and Compound G and LiQ were simultaneously vacuum deposited at a weight ratio of 1:1 to form a 300 Å-thick electron transport layer. On the electron transport layer, a cathode was formed by sequentially vacuum-depositing 15 Å of LiQ and 1200 Å of Al, manufacturing an organic light emitting diode.

The organic light emitting diode was manufactured to have a structure of ITO/Compound A (3% NDP-9 doping, 100 Å)/Compound A (1350 Å)/Compound E (350 Å)/EML [90 wt % of host (Compound A-12: Compound B-239=4:6 w/w): 10 wt % of PhGD](330 Å)/Compound F (50 Å)/Compound G:LiQ (300 Å)/LiQ (15 Å)/Al (1200 Å).

Compound E: N,N-bis(9,9-dimethyl-9H-fluoren-4-yl)-9,9-spirobi(fluorene)-2-amine

Compound F: 2-[3′-(9,9-Dimethyl-9H-fluoren-2-yl)[1,1′-biphenyl]-3-yl]-4,6-diphenyl-1,3,5-triazine

Compound G: 2-[4-[4-(4′-Cyano-1,1′-biphenyl-4-yl)-1-naphthyl]phenyl]-4,6-diphenyl-1,3,5-triazine

### Examples 11 to 18 and Comparative Examples 7 to 12

Diodes of Examples 11 to 18 and Comparative Examples 7 to 12 were manufactured in the same manner as in Example 2, except that the host and composition were changed as described in Tables 5 and 6.

### Evaluation

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Luminous efficiency (cd/A) at the same current density (10 mA/cm²) was calculated by using the luminance and current density from (1) and (2) above.

The relative values based on the luminous efficiency of Comparative Example 1 are shown in Table 3.

The relative values based on the luminous efficiency of Comparative Example 4 are shown in Table 4.

The relative values based on the luminous efficiency of Comparative Example 7 are shown in Table 5.

The relative values based on the luminous efficiency of Comparative Example 10 are shown in Table 6.

### (4) Measurement of Life-Span

The organic light emitting diodes were measured with respect to T95 life-spans by emitting light at initial luminance (cd/m²) of 24,000 cd/m² and measuring luminance decreases over time to obtain when the luminance decreased down to 95% of the initial luminance as T95 life-span.

The relative values based on the T95 life-span of Comparative Example 1 are shown in Table 3.

The relative values based on the T95 life-span of Comparative Example 4 are shown in Table 4.

The relative values based on the T95 life-span of Comparative Example 7 are shown in Table 5.

The relative values based on the T95 life-span of Comparative Example 10 are shown in Table 6.

### (5) Measurement of Driving Voltage

A current-voltage meter (Keithley 2400) was used to measure a driving voltage of each device at 15 mA/cm².

The relative values based on the driving voltage of Comparative Example 1 are shown in Table 3.

The relative values based on the driving voltage of Comparative Example 4 are shown in Table 4.

The relative values based on the driving voltage of Comparative Example 7 are shown in Table 5.

The relative values based on the driving voltage of Comparative Example 10 are shown in Table 6.

TABLE 3

| | | Driving | Luminous | Life-span |
|---|---|---|---|---|
| | | voltage | efficiency | T95 |
| | Host | (%) | (%) | (%) |
| Example 1 | Compound A-74 | 88 | 112 | 190 |
| Example 2 | Compound A-76 | 85 | 116 | 230 |
| Example 3 | Compound A-84 | 93 | 108 | 250 |
| Comparative | Compound C1 | 100 | 100 | 100 |
| Example 1 | | | | |
| Comparative | Compound C2 | 96 | 104 | 140 |
| Example 2 | | | | |
| Comparative | Compound C3 | 109 | 85 | 10 |
| Example 3 | | | | |

TABLE 4

| | | Driving | Luminous | Life-span |
|---|---|---|---|---|
| | | voltage | efficiency | T95 |
| | Host | (%) | (%) | (%) |
| Example 4 | Compound A-12 | 92 | 110 | 165 |
| Example 5 | Compound A-20 | 85 | 113 | 155 |
| Example 6 | Compound A-89 | 80 | 115 | 130 |
| Example 7 | Compound A-24 | 84 | 113 | 150 |
| Example 8 | Compound A-51 | 91 | 111 | 135 |
| Example 9 | Compound A-57 | 85 | 113 | 135 |
| Comparative | Compound C4 | 100 | 100 | 100 |
| Example 4 | | | | |
| Comparative | Compound C5 | 92 | 110 | 125 |
| Example 5 | | | | |
| Comparative | Compound C6 | 92 | 110 | 60 |
| Example 6 | | | | |

TABLE 5

| | | | Driving | Luminous |
|---|---|---|---|---|
| | First | Second | voltage | efficiency |
| | host | host | (%) | (%) |
| Example 10 | Compound A-12 | B-239 | 95 | 120 |
| Example 11 | Compound A-20 | B-239 | 90 | 122 |
| Example 12 | Compound A-89 | B-239 | 85 | 123 |
| Example 13 | Compound A-24 | B-239 | 89 | 122 |
| Example 14 | Compound A-51 | B-239 | 94 | 120 |
| Example 15 | Compound A-57 | B-239 | 96 | 124 |
| Comparative | Compound C1 | B-239 | 100 | 100 |
| Example 7 | | | | |
| Comparative | Compound C2 | B-239 | 97 | 110 |
| Example 8 | | | | |
| Comparative | Compound C3 | B-239 | 110 | 80 |
| Example 9 | | | | |

TABLE 6

| | | | Driving | Life-span |
|---|---|---|---|---|
| | First | Second | voltage | T95 |
| | host | host | (%) | (%) |
| Example 16 | Compound A-74 | B-239 | 96 | 170 |
| Example 17 | Compound A-76 | B-239 | 94 | 190 |
| Example 18 | Compound A-84 | B-239 | 95 | 210 |
| Comparative | Compound C4 | B-239 | 100 | 100 |
| Example 10 | | | | |
| Comparative | Compound C5 | B-239 | 99 | 110 |
| Example 11 | | | | |
| Comparative | Compound C6 | B-239 | 99 | 40 |
| Example 12 | | | | |

Referring to Table 3 and Table 4, the organic light emitting diodes to which the compounds according to Examples of the present invention were applied exhibited significantly improved driving voltage, efficiency, and life-span characteristics compared to the organic light emitting diodes according to Comparative Examples.

In particular, referring to Tables 5 and 6, the organic light emitting diode to which the compositions including the compounds according to Examples of the present invention were applied also exhibited improved driving voltage and efficiency, or life-span characteristics.

While this invention has been described in connection with what is presently considered to be practical example embodiments, it is to be understood that the invention is not limited to the disclosed embodiments. On the contrary, it is intended to cover various modifications and equivalent arrangements included within the spirit and scope of the appended claims.

1. A compound for an organic optoelectronic device, the compound being represented by Chemical Formula 1: 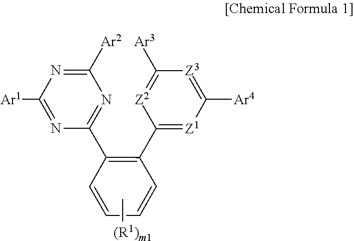
wherein, in Chemical Formula 1,
Z¹ to Z³ are each independently N or CR^{a}, provided that at least two of Z¹ to Z³ are N,
Ar¹ to Ar⁴ are each independently hydrogen, deuterium, a cyano group, a halogen, a substituted or unsubstituted C6 to C30 aryl group, a substituted or unsubstituted carbazolyl group, a substituted or unsubstituted dibenzofuranyl group, a substituted or unsubstituted dibenzothiophenyl group, or a combination thereof, and
one of Ar¹ to Ar⁴ is a group represented by Chemical Formula A, 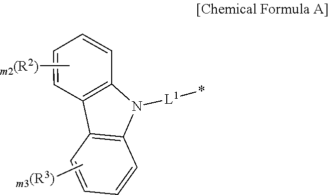
L¹ is a single bond or a substituted or unsubstituted C6 to C20 arylene group,
R^{a} and R¹ to R³ are each independently hydrogen, deuterium, a cyano group, a halogen, a substituted or unsubstituted C1 to C30 alkyl group, a substituted or unsubstituted C6 to C30 aryl group, or a substituted or unsubstituted C2 to C30 heterocyclic group,
m1 to m3 are each independently an integer of 1 to 4,
* is a linking point, and
Chemical formula 1 satisfies at least one of the following conditions (i) to (iii):
(i) at least one of the remaining substituents not substituted by Chemical Formula A among Ar¹ to Ar⁴ is a substituted or unsubstituted C10 to C30 aryl group;
(ii) at least one of R² and R³ is a substituted or unsubstituted C10 to C30 aryl group; or
(iii) R¹ is a substituted or unsubstituted C6 to C30 aryl group.

2. The compound for an organic optoelectronic device as claimed in claim 1, wherein:
the compound is represented by Chemical Formula 1A or Chemical Formula 1B: 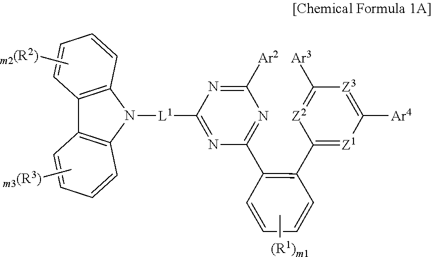
in Chemical Formula 1A, Z¹ to Z³, Ar¹ to Ar⁴, L¹, R¹ to R³, and m1 to m3 are defined the same as those of Chemical Formula 1, and
Chemical Formula 1A satisfies at least one of the following conditions (iii) to (v):
(iii) at least one of Ar² to Ar⁴ is a substituted or unsubstituted C10 to C30 aryl group;
(iv) at least one of R² and R³ is a substituted or unsubstituted C10 to C30 aryl group; or
(v) R¹ is a substituted or unsubstituted C6 to C30 aryl group; 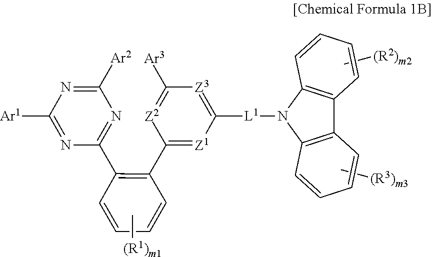
in Chemical Formula 1B, Z¹ to Z³, Ar¹ to Ar⁴, L¹, R¹ to R³ and m1 to m3 are defined the same as those of Chemical Formula 1, and
Chemical Formula 1B satisfies at least one of the following conditions (vi) to (viii):
(vi) at least one of Ar¹ to Ar³ is a substituted or unsubstituted C10 to C30 aryl group;
(vii) at least one of R² and R³ is a substituted or unsubstituted C10 to C30 aryl group; or
(viii) R¹ is a substituted or unsubstituted C6 to C30 aryl group.

3. The compound for an organic optoelectronic device as claimed in claim 2, wherein:
the compound is represented by Chemical Formula 1A, and
Chemical Formula 1A is represented by one of Chemical Formula 1A-I, Chemical Formula 1A-II and Chemical Formula 1A-III: 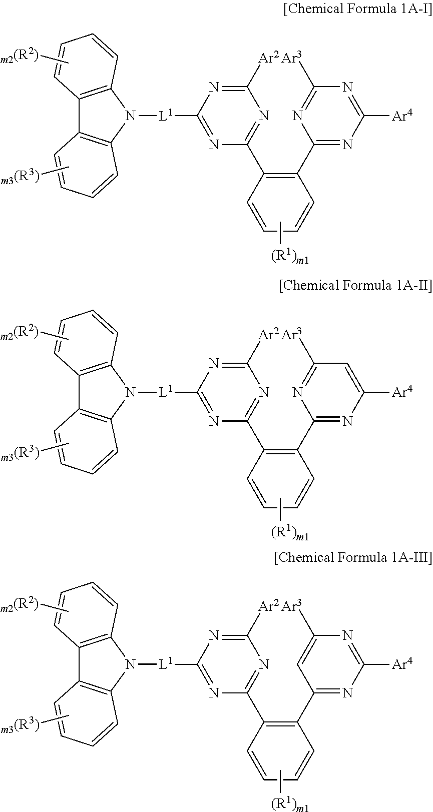
in Chemical Formula 1A-I, Chemical Formula 1A-II, and Chemical Formula 1A-III, Ar² to Ar⁴, L¹, R¹ to R³, and m1 to m3 are defined the same as those of Chemical Formula 1A.

4. The compound for an organic optoelectronic device as claimed in claim 2, wherein:
the compound is represented by Chemical Formula 1B, and
Chemical Formula 1B is represented by Chemical Formula 1B-II or Chemical Formula 1B-III: 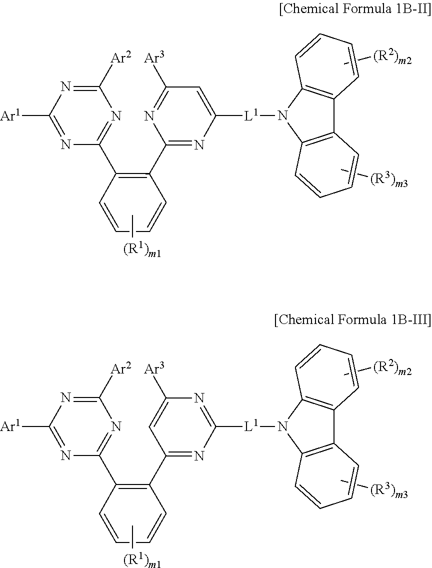
in Chemical Formula 1B-II and Chemical Formula 1B-III, Ar¹ to Ar³, L¹, R¹ to R³, and m1 to m3 are defined the same as those of Chemical Formula 1B.

5. The compound for an organic optoelectronic device as claimed in claim 1, wherein:
Ar¹ to Ar⁴ are each independently a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted triphenylene group, a substituted or unsubstituted dibenzofuranyl group, a substituted or unsubstituted dibenzothiophenyl group, or a substituted or unsubstituted carbazolyl group,
at least one of Ar¹ to Ar⁴ is represented by Chemical Formula A, and
Chemical Formula 1 satisfies at least one of the following conditions (ix) to (xi):
(ix) at least one of the remaining substituents not substituted by Chemical Formula A among Ar¹ to Ar⁴ is a substituted or unsubstituted biphenyl group, a substituted or unsubstituted terphenyl group, a substituted or unsubstituted naphthyl group, a substituted or unsubstituted fluorenyl group, or a substituted or unsubstituted triphenylene group;
(x) at least one of R² and R³ is a substituted or unsubstituted biphenyl group, a substituted or unsubstituted terphenyl group, a substituted or unsubstituted naphthyl group, a substituted or unsubstituted fluorenyl group, or a substituted or unsubstituted triphenylene group; or
(xi) R¹ is a substituted or unsubstituted C6 to C12 aryl group.

6. The compound for an organic optoelectronic device as claimed in claim 1, wherein R¹ to R³ are each independently hydrogen, deuterium, a substituted or unsubstituted phenyl group, or a substituted or unsubstituted biphenyl group.

7. The compound for an organic optoelectronic device as claimed in claim 1, wherein the compound is a compound of Group 1:

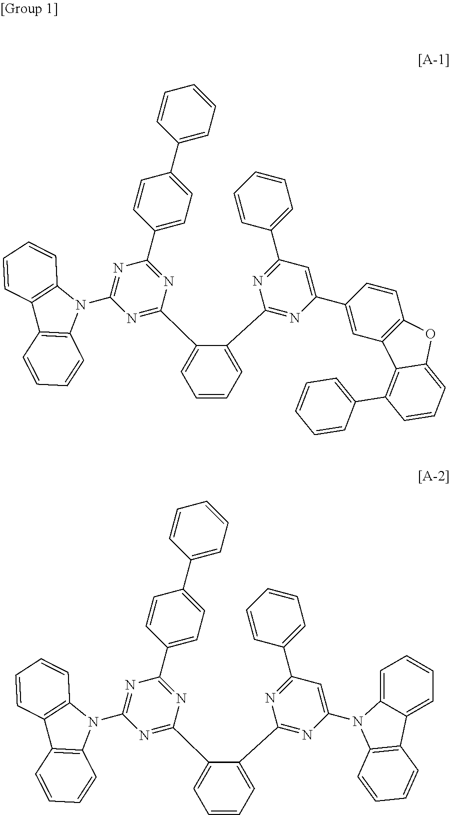 [SMILES: c1ccc(-c2ccc(-c3nc(-c4ccccc4-c4nc(-c5ccccc5)cc(-c5ccc6oc7cccc(-c8ccccc8)c7c6c5)n4)nc(-n4c5ccccc5c5ccccc54)n3)cc2)cc1 ; c1ccc(-c2ccc(-c3nc(-c4ccccc4-c4nc(-c5ccccc5)cc(-n5c6ccccc6c6ccccc65)n4)nc(-n4c5ccccc5c5ccccc54)n3)cc2)cc1]

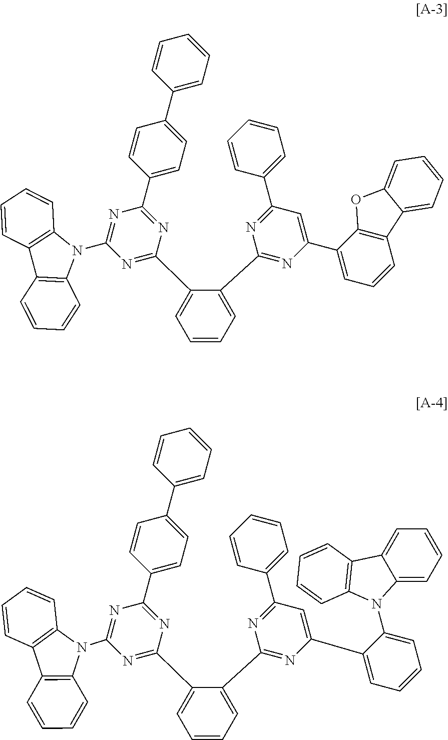 [SMILES: c1ccc(-c2ccc(-c3nc(-c4ccccc4-c4nc(-c5ccccc5)cc(-c5cccc6c5oc5ccccc56)n4)nc(-n4c5ccccc5c5ccccc54)n3)cc2)cc1 ; c1ccc(-c2ccc(-c3nc(-c4ccccc4-c4nc(-c5ccccc5)cc(-c5ccccc5-n5c6ccccc6c6ccccc65)n4)nc(-n4c5ccccc5c5ccccc54)n3)cc2)cc1]

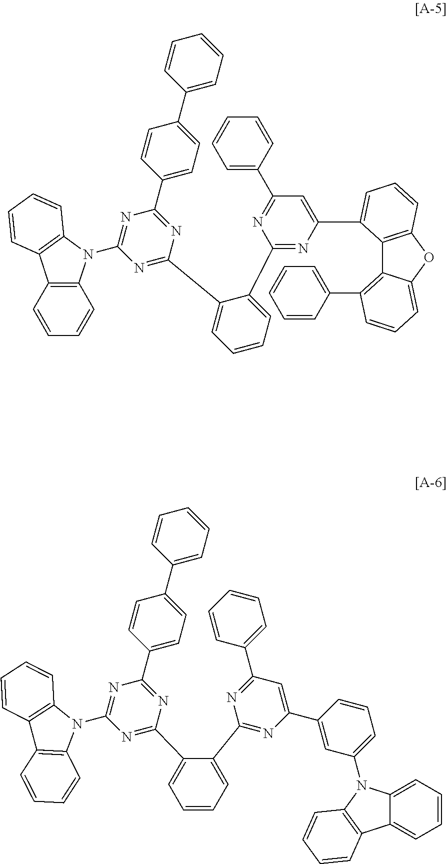 [SMILES: c1ccc(-c2ccc(-c3nc(-c4ccccc4-c4nc(-c5ccccc5)cc(-c5cccc6oc7cccc(-c8ccccc8)c7c56)n4)nc(-n4c5ccccc5c5ccccc54)n3)cc2)cc1 ; c1ccc(-c2ccc(-c3nc(-c4ccccc4-c4nc(-c5ccccc5)cc(-c5cccc(-n6c7ccccc7c7ccccc76)c5)n4)nc(-n4c5ccccc5c5ccccc54)n3)cc2)cc1]

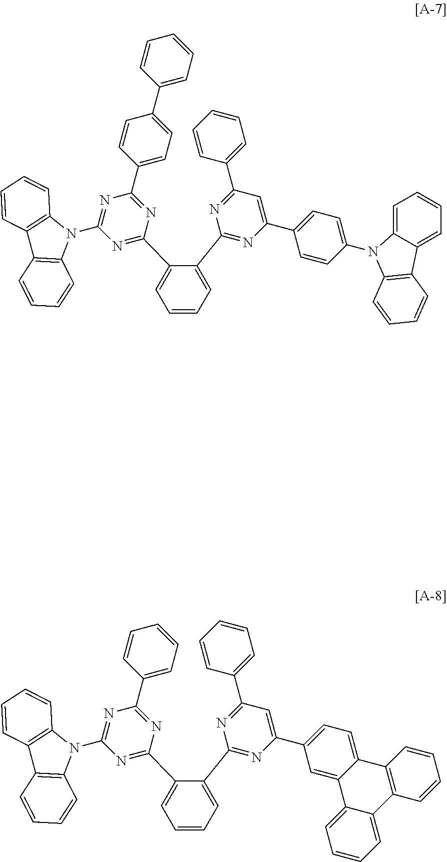 [SMILES: c1ccc(-c2ccc(-c3nc(-c4ccccc4-c4nc(-c5ccccc5)cc(-c5ccc(-n6c7ccccc7c7ccccc76)cc5)n4)nc(-n4c5ccccc5c5ccccc54)n3)cc2)cc1 ; c1ccc(-c2cc(-c3ccc4c5ccccc5c5ccccc5c4c3)nc(-c3ccccc3-c3nc(-c4ccccc4)nc(-n4c5ccccc5c5ccccc54)n3)n2)cc1]

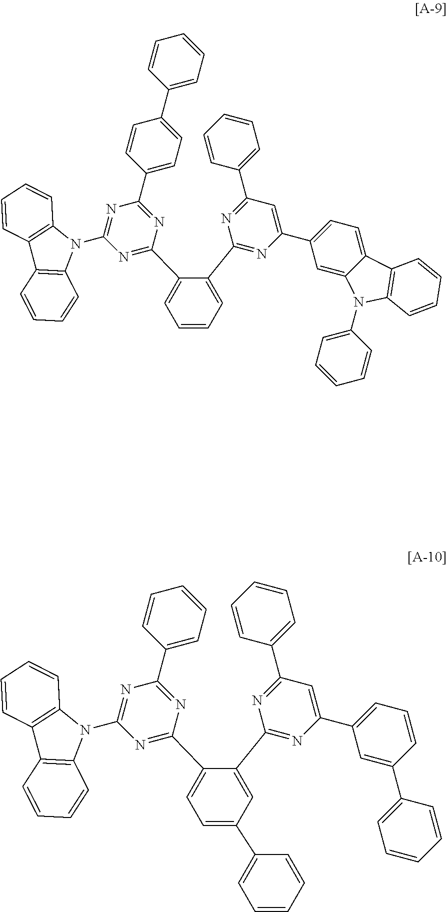 [SMILES: c1ccc(-c2ccc(-c3nc(-c4ccccc4-c4nc(-c5ccccc5)cc(-c5ccc6c7ccccc7n(-c7ccccc7)c6c5)n4)nc(-n4c5ccccc5c5ccccc54)n3)cc2)cc1 ; c1ccc(-c2cccc(-c3cc(-c4ccccc4)nc(-c4cc(-c5ccccc5)ccc4-c4nc(-c5ccccc5)nc(-n5c6ccccc6c6ccccc65)n4)n3)c2)cc1]

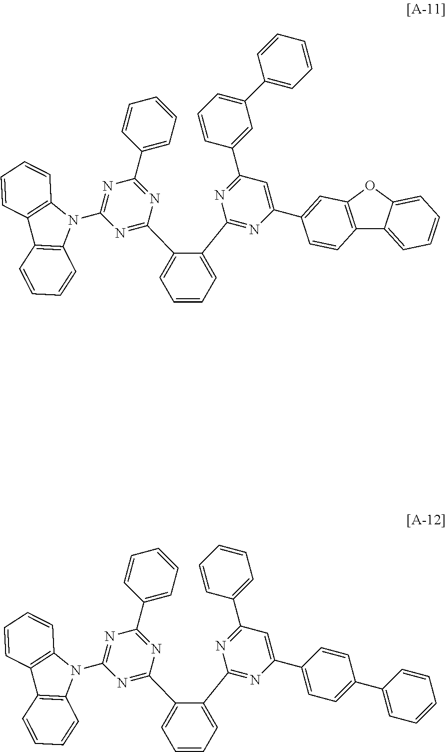 [SMILES: c1ccc(-c2cccc(-c3cc(-c4ccc5c(c4)oc4ccccc45)nc(-c4ccccc4-c4nc(-c5ccccc5)nc(-n5c6ccccc6c6ccccc65)n4)n3)c2)cc1 ; c1ccc(-c2ccc(-c3cc(-c4ccccc4)nc(-c4ccccc4-c4nc(-c5ccccc5)nc(-n5c6ccccc6c6ccccc65)n4)n3)cc2)cc1]

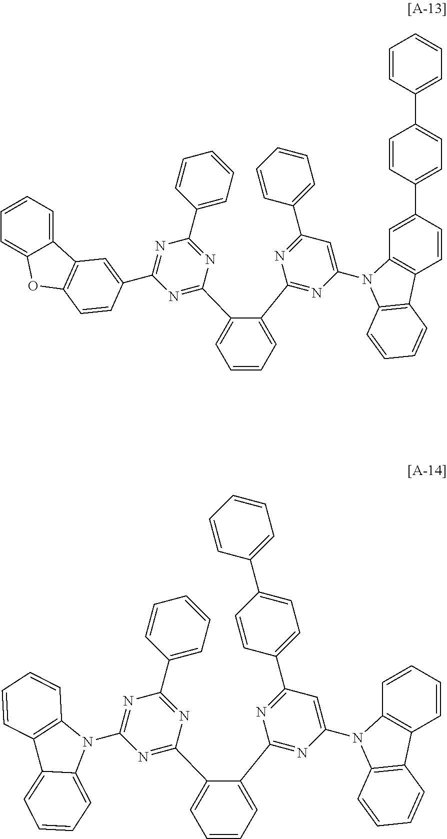 [SMILES: c1ccc(-c2ccc(-c3ccc4c5ccccc5n(-c5cc(-c6ccccc6)nc(-c6ccccc6-c6nc(-c7ccccc7)nc(-c7ccc8oc9ccccc9c8c7)n6)n5)c4c3)cc2)cc1 ; c1ccc(-c2ccc(-c3cc(-n4c5ccccc5c5ccccc54)nc(-c4ccccc4-c4nc(-c5ccccc5)nc(-n5c6ccccc6c6ccccc65)n4)n3)cc2)cc1]

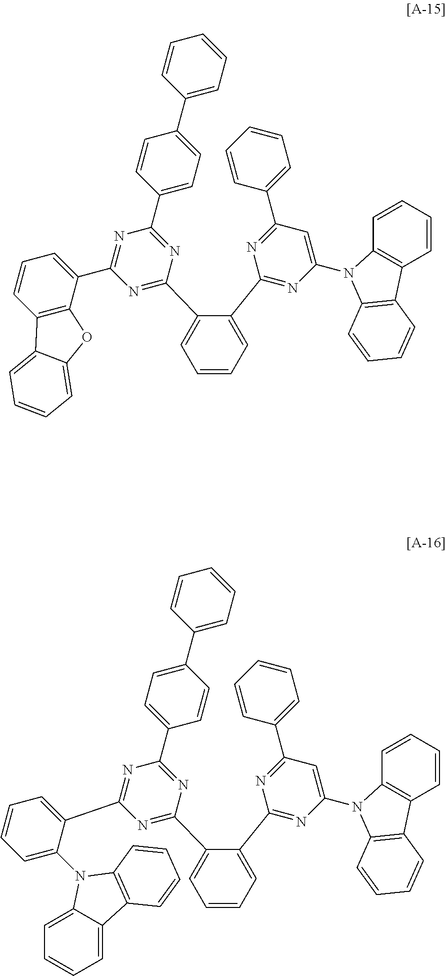 [SMILES: c1ccc(-c2ccc(-c3nc(-c4ccccc4-c4nc(-c5ccccc5)cc(-n5c6ccccc6c6ccccc65)n4)nc(-c4cccc5c4oc4ccccc45)n3)cc2)cc1 ; c1ccc(-c2ccc(-c3nc(-c4ccccc4-c4nc(-c5ccccc5)cc(-n5c6ccccc6c6ccccc65)n4)nc(-c4ccccc4-n4c5ccccc5c5ccccc54)n3)cc2)cc1]

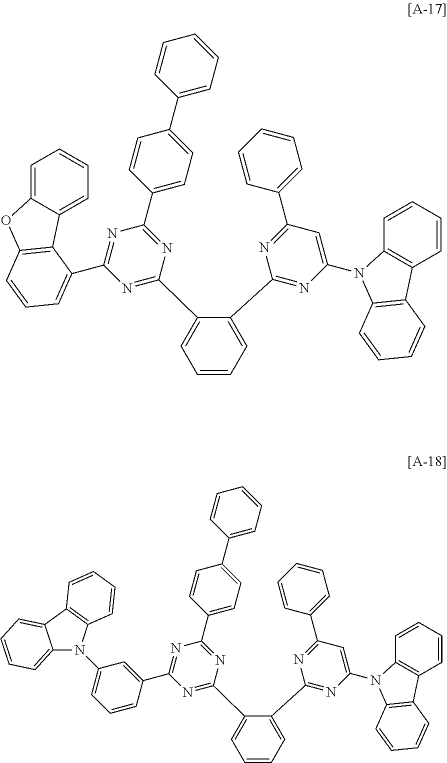 [SMILES: c1ccc(-c2ccc(-c3nc(-c4ccccc4-c4nc(-c5ccccc5)cc(-n5c6ccccc6c6ccccc65)n4)nc(-c4cccc5oc6ccccc6c45)n3)cc2)cc1 ; c1ccc(-c2ccc(-c3nc(-c4cccc(-n5c6ccccc6c6ccccc65)c4)nc(-c4ccccc4-c4nc(-c5ccccc5)cc(-n5c6ccccc6c6ccccc65)n4)n3)cc2)cc1]

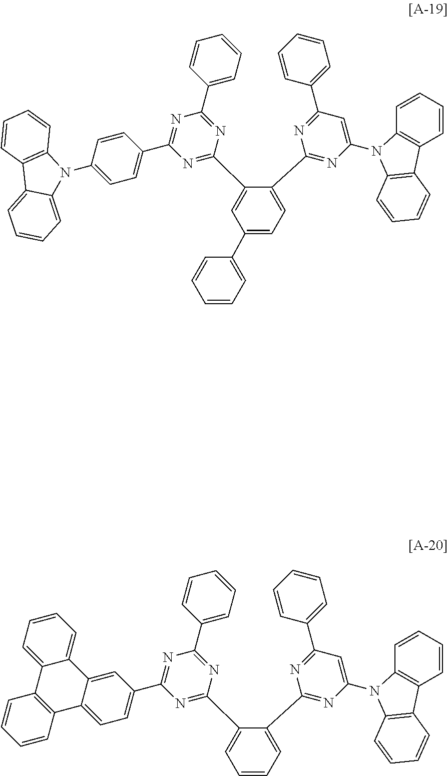 [SMILES: c1ccc(-c2ccc(-c3nc(-c4ccccc4)cc(-n4c5ccccc5c5ccccc54)n3)c(-c3nc(-c4ccccc4)nc(-c4ccc(-n5c6ccccc6c6ccccc65)cc4)n3)c2)cc1 ; c1ccc(-c2cc(-n3c4ccccc4c4ccccc43)nc(-c3ccccc3-c3nc(-c4ccccc4)nc(-c4ccc5c6ccccc6c6ccccc6c5c4)n3)n2)cc1]

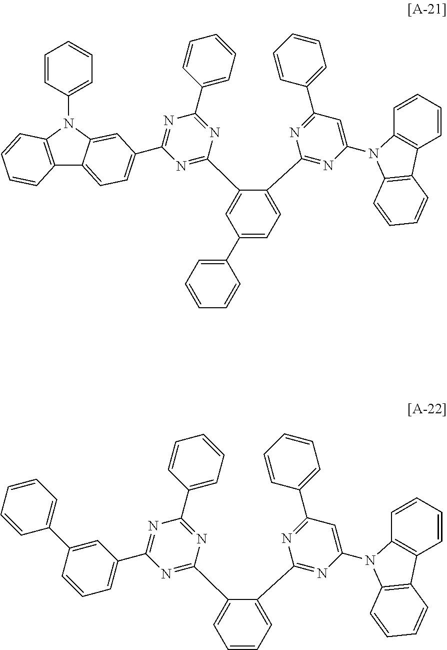 [SMILES: c1ccc(-c2ccc(-c3nc(-c4ccccc4)cc(-n4c5ccccc5c5ccccc54)n3)c(-c3nc(-c4ccccc4)nc(-c4ccc5c6ccccc6n(-c6ccccc6)c5c4)n3)c2)cc1 ; c1ccc(-c2cccc(-c3nc(-c4ccccc4)nc(-c4ccccc4-c4nc(-c5ccccc5)cc(-n5c6ccccc6c6ccccc65)n4)n3)c2)cc1]

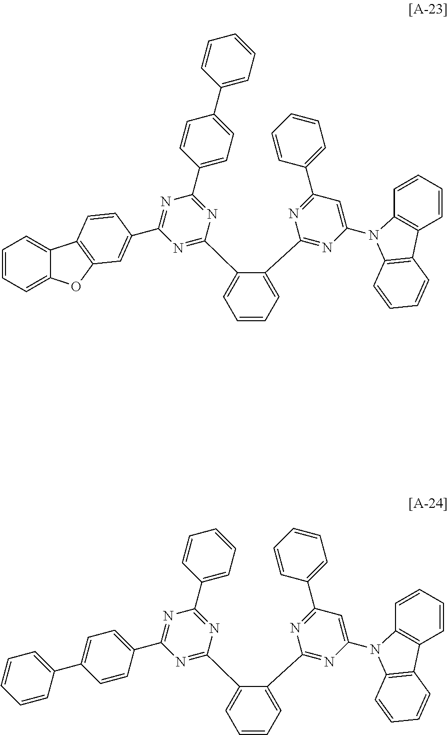 [SMILES: c1ccc(-c2ccc(-c3nc(-c4ccc5c(c4)oc4ccccc45)nc(-c4ccccc4-c4nc(-c5ccccc5)cc(-n5c6ccccc6c6ccccc65)n4)n3)cc2)cc1 ; c1ccc(-c2ccc(-c3nc(-c4ccccc4)nc(-c4ccccc4-c4nc(-c5ccccc5)cc(-n5c6ccccc6c6ccccc65)n4)n3)cc2)cc1]

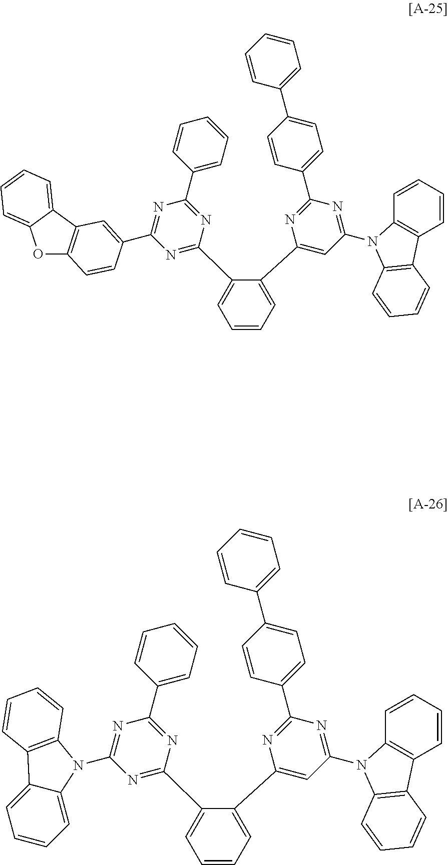 [SMILES: c1ccc(-c2ccc(-c3nc(-c4ccccc4-c4nc(-c5ccccc5)nc(-c5ccc6oc7ccccc7c6c5)n4)cc(-n4c5ccccc5c5ccccc54)n3)cc2)cc1 ; c1ccc(-c2ccc(-c3nc(-c4ccccc4-c4nc(-c5ccccc5)nc(-n5c6ccccc6c6ccccc65)n4)cc(-n4c5ccccc5c5ccccc54)n3)cc2)cc1]

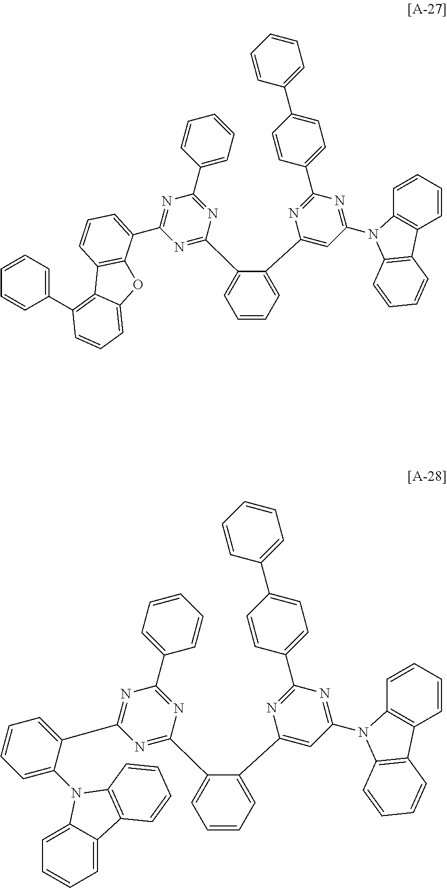 [SMILES: c1ccc(-c2ccc(-c3nc(-c4ccccc4-c4nc(-c5ccccc5)nc(-c5cccc6c5oc5cccc(-c7ccccc7)c56)n4)cc(-n4c5ccccc5c5ccccc54)n3)cc2)cc1 ; c1ccc(-c2ccc(-c3nc(-c4ccccc4-c4nc(-c5ccccc5)nc(-c5ccccc5-n5c6ccccc6c6ccccc65)n4)cc(-n4c5ccccc5c5ccccc54)n3)cc2)cc1]

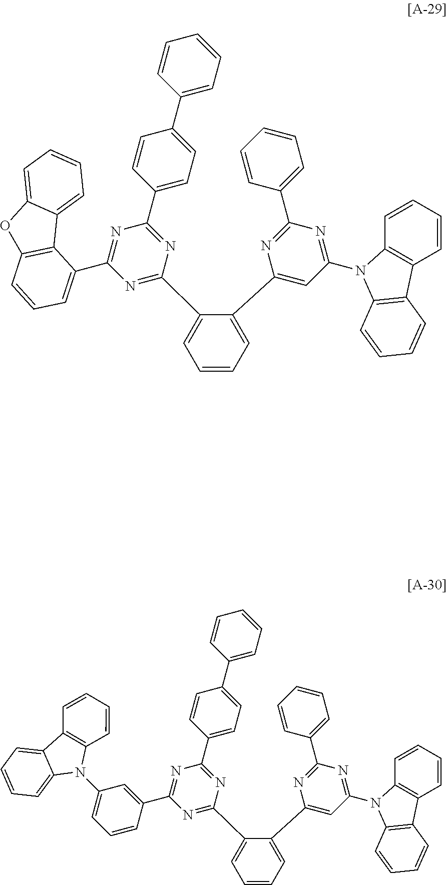 [SMILES: c1ccc(-c2ccc(-c3nc(-c4ccccc4-c4cc(-n5c6ccccc6c6ccccc65)nc(-c5ccccc5)n4)nc(-c4cccc5oc6ccccc6c45)n3)cc2)cc1 ; c1ccc(-c2ccc(-c3nc(-c4cccc(-n5c6ccccc6c6ccccc65)c4)nc(-c4ccccc4-c4cc(-n5c6ccccc6c6ccccc65)nc(-c5ccccc5)n4)n3)cc2)cc1]

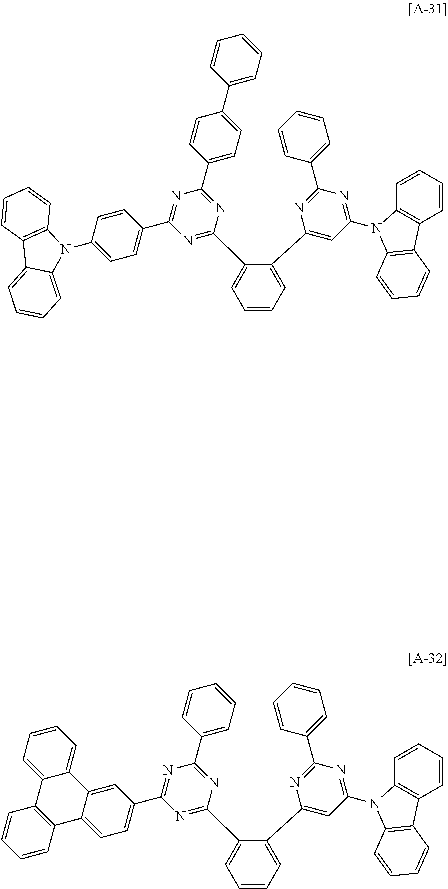 [SMILES: c1ccc(-c2ccc(-c3nc(-c4ccc(-n5c6ccccc6c6ccccc65)cc4)nc(-c4ccccc4-c4cc(-n5c6ccccc6c6ccccc65)nc(-c5ccccc5)n4)n3)cc2)cc1 ; c1ccc(-c2nc(-c3ccccc3-c3nc(-c4ccccc4)nc(-c4ccc5c6ccccc6c6ccccc6c5c4)n3)cc(-n3c4ccccc4c4ccccc43)n2)cc1]

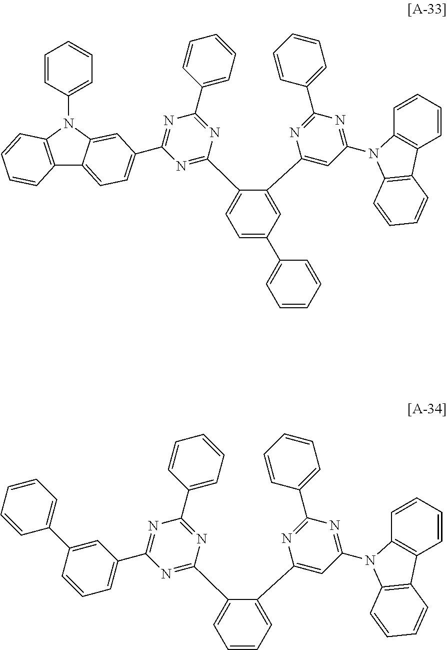 [SMILES: c1ccc(-c2ccc(-c3nc(-c4ccccc4)nc(-c4ccc5c6ccccc6n(-c6ccccc6)c5c4)n3)c(-c3cc(-n4c5ccccc5c5ccccc54)nc(-c4ccccc4)n3)c2)cc1 ; c1ccc(-c2cccc(-c3nc(-c4ccccc4)nc(-c4ccccc4-c4cc(-n5c6ccccc6c6ccccc65)nc(-c5ccccc5)n4)n3)c2)cc1]

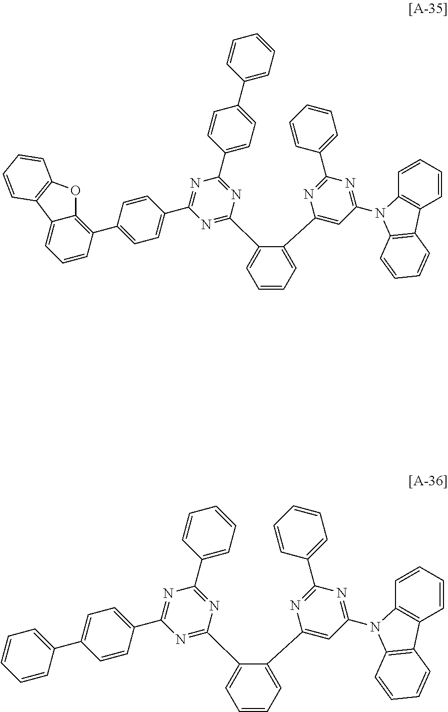 [SMILES: c1ccc(-c2ccc(-c3nc(-c4ccc(-c5cccc6c5oc5ccccc56)cc4)nc(-c4ccccc4-c4cc(-n5c6ccccc6c6ccccc65)nc(-c5ccccc5)n4)n3)cc2)cc1 ; c1ccc(-c2ccc(-c3nc(-c4ccccc4)nc(-c4ccccc4-c4cc(-n5c6ccccc6c6ccccc65)nc(-c5ccccc5)n4)n3)cc2)cc1]

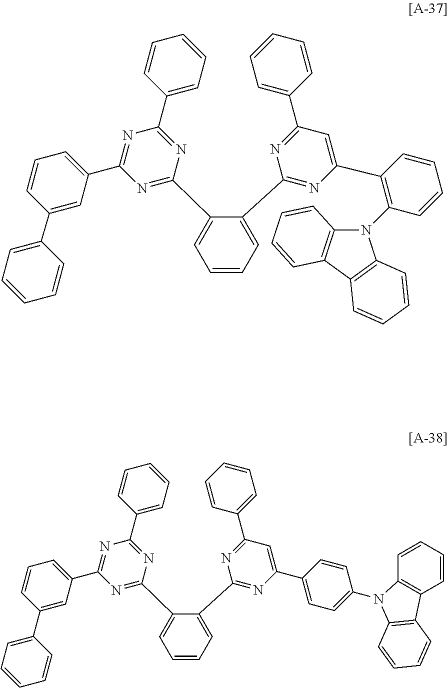 [SMILES: c1ccc(-c2cccc(-c3nc(-c4ccccc4)nc(-c4ccccc4-c4nc(-c5ccccc5)cc(-c5ccccc5-n5c6ccccc6c6ccccc65)n4)n3)c2)cc1 ; c1ccc(-c2cccc(-c3nc(-c4ccccc4)nc(-c4ccccc4-c4nc(-c5ccccc5)cc(-c5ccc(-n6c7ccccc7c7ccccc76)cc5)n4)n3)c2)cc1]

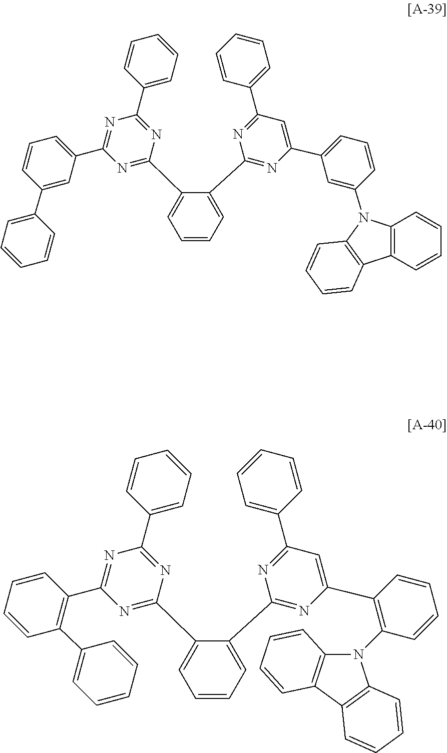 [SMILES: c1ccc(-c2cccc(-c3nc(-c4ccccc4)nc(-c4ccccc4-c4nc(-c5ccccc5)cc(-c5cccc(-n6c7ccccc7c7ccccc76)c5)n4)n3)c2)cc1 ; c1ccc(-c2cc(-c3ccccc3-n3c4ccccc4c4ccccc43)nc(-c3ccccc3-c3nc(-c4ccccc4)nc(-c4ccccc4-c4ccccc4)n3)n2)cc1]

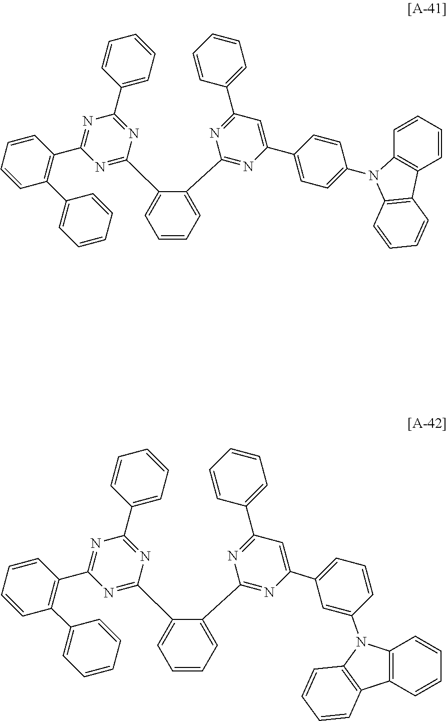 [SMILES: c1ccc(-c2cc(-c3ccc(-n4c5ccccc5c5ccccc54)cc3)nc(-c3ccccc3-c3nc(-c4ccccc4)nc(-c4ccccc4-c4ccccc4)n3)n2)cc1 ; c1ccc(-c2cc(-c3cccc(-n4c5ccccc5c5ccccc54)c3)nc(-c3ccccc3-c3nc(-c4ccccc4)nc(-c4ccccc4-c4ccccc4)n3)n2)cc1]

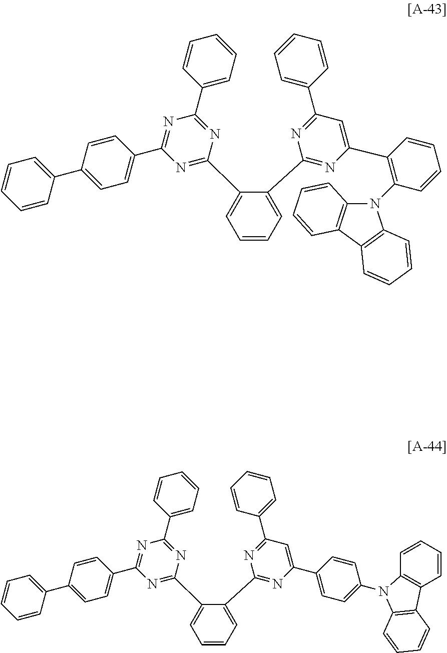 [SMILES: c1ccc(-c2ccc(-c3nc(-c4ccccc4)nc(-c4ccccc4-c4nc(-c5ccccc5)cc(-c5ccccc5-n5c6ccccc6c6ccccc65)n4)n3)cc2)cc1 ; c1ccc(-c2ccc(-c3nc(-c4ccccc4)nc(-c4ccccc4-c4nc(-c5ccccc5)cc(-c5ccc(-n6c7ccccc7c7ccccc76)cc5)n4)n3)cc2)cc1]

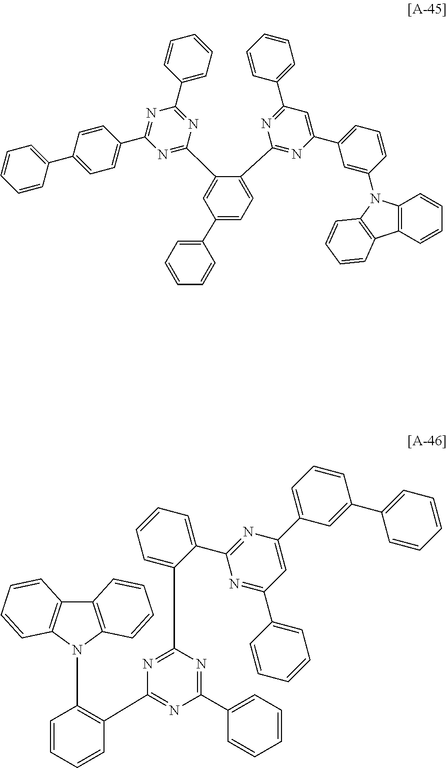 [SMILES: c1ccc(-c2ccc(-c3nc(-c4ccccc4)nc(-c4cc(-c5ccccc5)ccc4-c4nc(-c5ccccc5)cc(-c5cccc(-n6c7ccccc7c7ccccc76)c5)n4)n3)cc2)cc1 ; c1ccc(-c2cccc(-c3cc(-c4ccccc4)nc(-c4ccccc4-c4nc(-c5ccccc5)nc(-c5ccccc5-n5c6ccccc6c6ccccc65)n4)n3)c2)cc1]

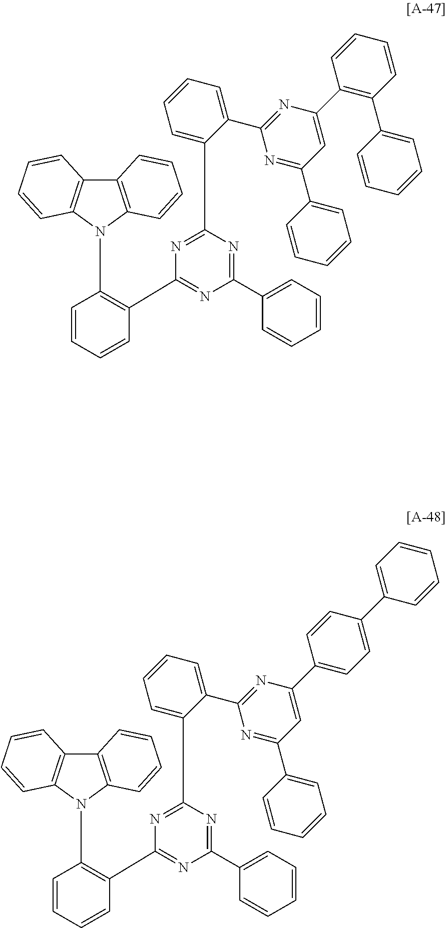 [SMILES: c1ccc(-c2cc(-c3ccccc3-c3ccccc3)nc(-c3ccccc3-c3nc(-c4ccccc4)nc(-c4ccccc4-n4c5ccccc5c5ccccc54)n3)n2)cc1 ; c1ccc(-c2ccc(-c3cc(-c4ccccc4)nc(-c4ccccc4-c4nc(-c5ccccc5)nc(-c5ccccc5-n5c6ccccc6c6ccccc65)n4)n3)cc2)cc1]

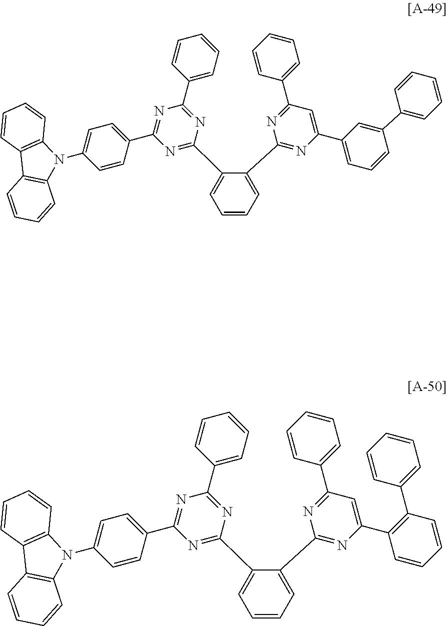 [SMILES: c1ccc(-c2cccc(-c3cc(-c4ccccc4)nc(-c4ccccc4-c4nc(-c5ccccc5)nc(-c5ccc(-n6c7ccccc7c7ccccc76)cc5)n4)n3)c2)cc1 ; c1ccc(-c2cc(-c3ccccc3-c3ccccc3)nc(-c3ccccc3-c3nc(-c4ccccc4)nc(-c4ccc(-n5c6ccccc6c6ccccc65)cc4)n3)n2)cc1]

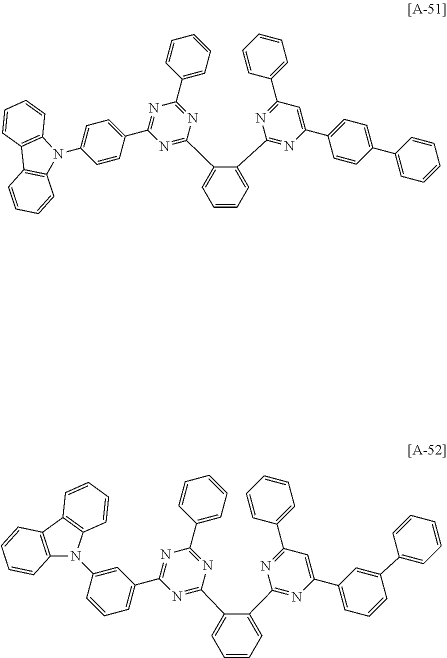 [SMILES: c1ccc(-c2ccc(-c3cc(-c4ccccc4)nc(-c4ccccc4-c4nc(-c5ccccc5)nc(-c5ccc(-n6c7ccccc7c7ccccc76)cc5)n4)n3)cc2)cc1 ; c1ccc(-c2cccc(-c3cc(-c4ccccc4)nc(-c4ccccc4-c4nc(-c5ccccc5)nc(-c5cccc(-n6c7ccccc7c7ccccc76)c5)n4)n3)c2)cc1]

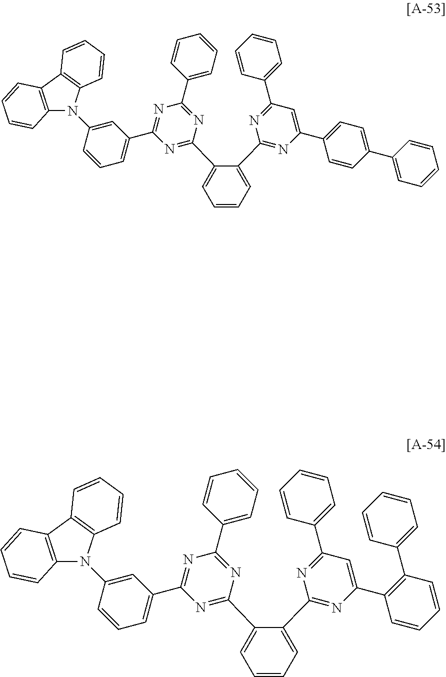 [SMILES: c1ccc(-c2ccc(-c3cc(-c4ccccc4)nc(-c4ccccc4-c4nc(-c5ccccc5)nc(-c5cccc(-n6c7ccccc7c7ccccc76)c5)n4)n3)cc2)cc1 ; c1ccc(-c2cc(-c3ccccc3-c3ccccc3)nc(-c3ccccc3-c3nc(-c4ccccc4)nc(-c4cccc(-n5c6ccccc6c6ccccc65)c4)n3)n2)cc1]

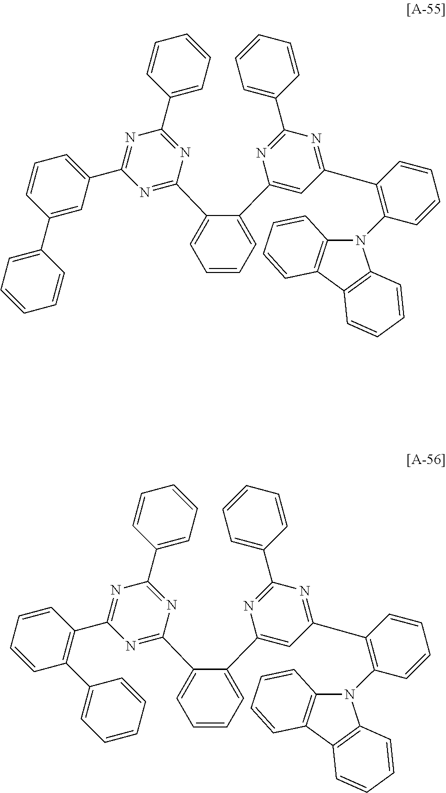 [SMILES: c1ccc(-c2cccc(-c3nc(-c4ccccc4)nc(-c4ccccc4-c4cc(-c5ccccc5-n5c6ccccc6c6ccccc65)nc(-c5ccccc5)n4)n3)c2)cc1 ; c1ccc(-c2nc(-c3ccccc3-c3nc(-c4ccccc4)nc(-c4ccccc4-c4ccccc4)n3)cc(-c3ccccc3-n3c4ccccc4c4ccccc43)n2)cc1]

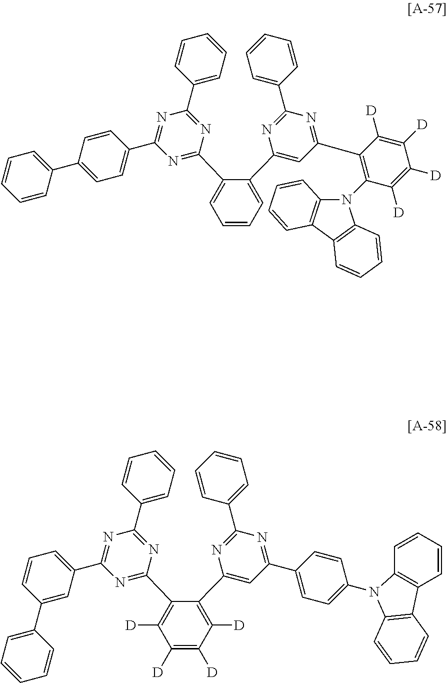 [SMILES: [2H]c1c([2H])c([2H])c(-n2c3ccccc3c3ccccc32)c(-c2cc(-c3ccccc3-c3nc(-c4ccccc4)nc(-c4ccc(-c5ccccc5)cc4)n3)nc(-c3ccccc3)n2)c1[2H] ; [2H]c1c([2H])c([2H])c(-c2nc(-c3ccccc3)nc(-c3cccc(-c4ccccc4)c3)n2)c(-c2cc(-c3ccc(-n4c5ccccc5c5ccccc54)cc3)nc(-c3ccccc3)n2)c1[2H]]

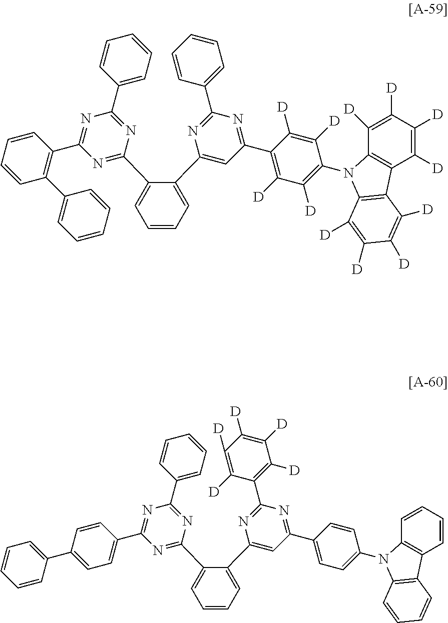 [SMILES: [2H]c1c([2H])c(-n2c3c([2H])c([2H])c([2H])c([2H])c3c3c([2H])c([2H])c([2H])c([2H])c32)c([2H])c([2H])c1-c1cc(-c2ccccc2-c2nc(-c3ccccc3)nc(-c3ccccc3-c3ccccc3)n2)nc(-c2ccccc2)n1 ; [2H]c1c([2H])c([2H])c(-c2nc(-c3ccc(-n4c5ccccc5c5ccccc54)cc3)cc(-c3ccccc3-c3nc(-c4ccccc4)nc(-c4ccc(-c5ccccc5)cc4)n3)n2)c([2H])c1[2H]]

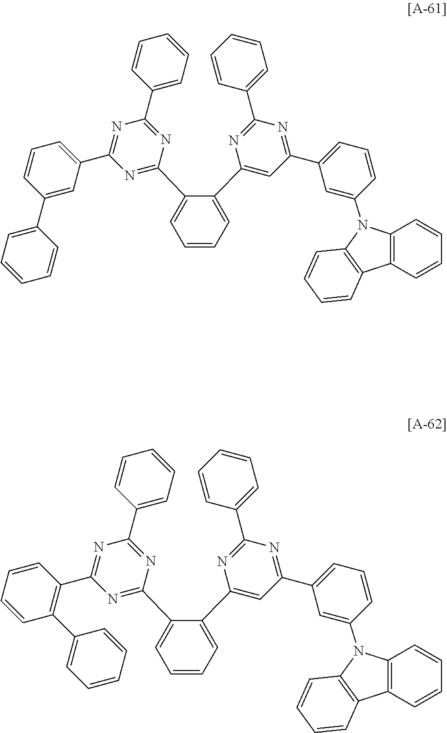 [SMILES: c1ccc(-c2cccc(-c3nc(-c4ccccc4)nc(-c4ccccc4-c4cc(-c5cccc(-n6c7ccccc7c7ccccc76)c5)nc(-c5ccccc5)n4)n3)c2)cc1 ; c1ccc(-c2nc(-c3cccc(-n4c5ccccc5c5ccccc54)c3)cc(-c3ccccc3-c3nc(-c4ccccc4)nc(-c4ccccc4-c4ccccc4)n3)n2)cc1]

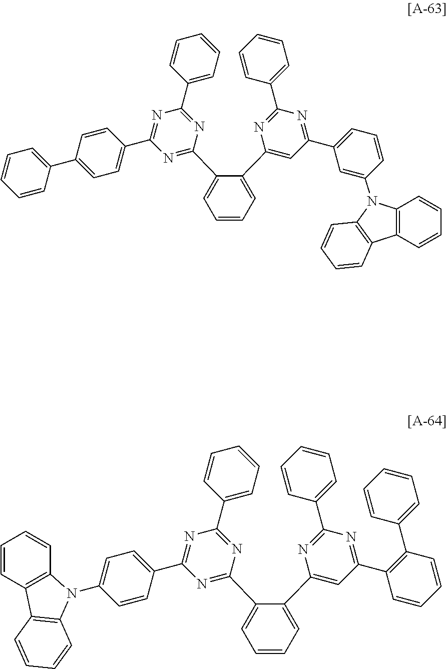 [SMILES: c1ccc(-c2ccc(-c3nc(-c4ccccc4)nc(-c4ccccc4-c4cc(-c5cccc(-n6c7ccccc7c7ccccc76)c5)nc(-c5ccccc5)n4)n3)cc2)cc1 ; c1ccc(-c2nc(-c3ccccc3-c3ccccc3)cc(-c3ccccc3-c3nc(-c4ccccc4)nc(-c4ccc(-n5c6ccccc6c6ccccc65)cc4)n3)n2)cc1]

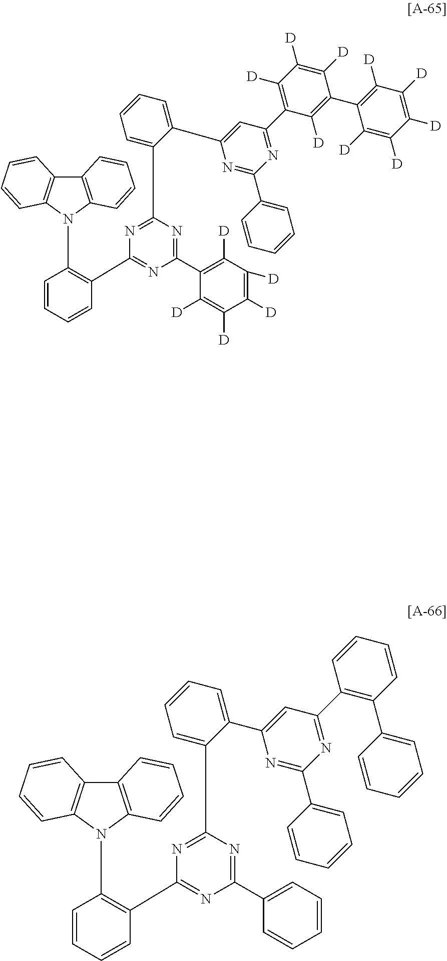 [SMILES: [2H]c1c([2H])c([2H])c(-c2nc(-c3ccccc3-c3cc(-c4c([2H])c([2H])c([2H])c(-c5c([2H])c([2H])c([2H])c([2H])c5[2H])c4[2H])nc(-c4ccccc4)n3)nc(-c3ccccc3-n3c4ccccc4c4ccccc43)n2)c([2H])c1[2H] ; c1ccc(-c2nc(-c3ccccc3-c3ccccc3)cc(-c3ccccc3-c3nc(-c4ccccc4)nc(-c4ccccc4-n4c5ccccc5c5ccccc54)n3)n2)cc1]

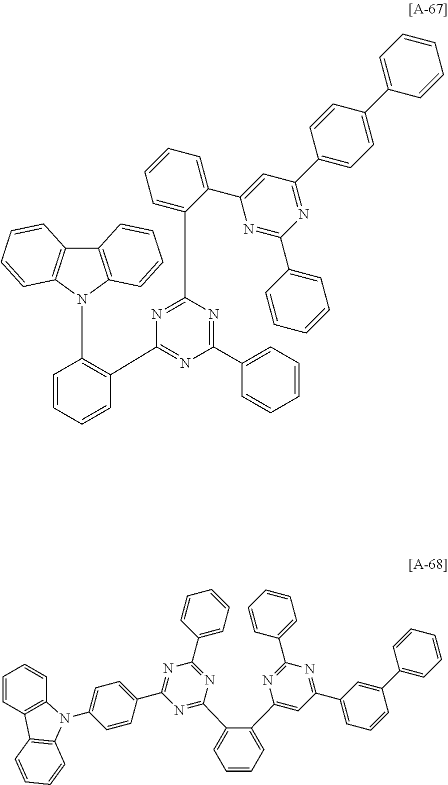 [SMILES: c1ccc(-c2ccc(-c3cc(-c4ccccc4-c4nc(-c5ccccc5)nc(-c5ccccc5-n5c6ccccc6c6ccccc65)n4)nc(-c4ccccc4)n3)cc2)cc1 ; c1ccc(-c2cccc(-c3cc(-c4ccccc4-c4nc(-c5ccccc5)nc(-c5ccc(-n6c7ccccc7c7ccccc76)cc5)n4)nc(-c4ccccc4)n3)c2)cc1]

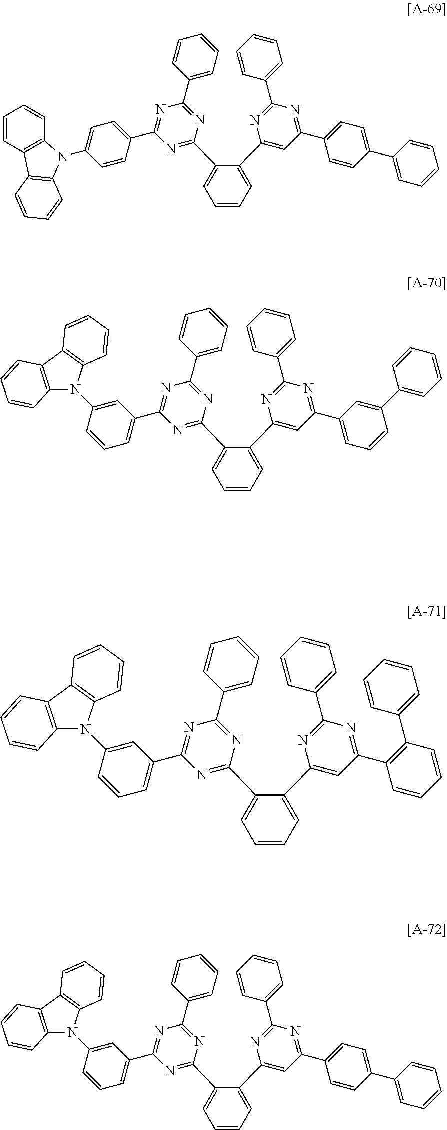 [SMILES: c1ccc(-c2ccc(-c3cc(-c4ccccc4-c4nc(-c5ccccc5)nc(-c5ccc(-n6c7ccccc7c7ccccc76)cc5)n4)nc(-c4ccccc4)n3)cc2)cc1 ; c1ccc(-c2cccc(-c3cc(-c4ccccc4-c4nc(-c5ccccc5)nc(-c5cccc(-n6c7ccccc7c7ccccc76)c5)n4)nc(-c4ccccc4)n3)c2)cc1 ; c1ccc(-c2nc(-c3ccccc3-c3ccccc3)cc(-c3ccccc3-c3nc(-c4ccccc4)nc(-c4cccc(-n5c6ccccc6c6ccccc65)c4)n3)n2)cc1 ; c1ccc(-c2ccc(-c3cc(-c4ccccc4-c4nc(-c5ccccc5)nc(-c5cccc(-n6c7ccccc7c7ccccc76)c5)n4)nc(-c4ccccc4)n3)cc2)cc1]

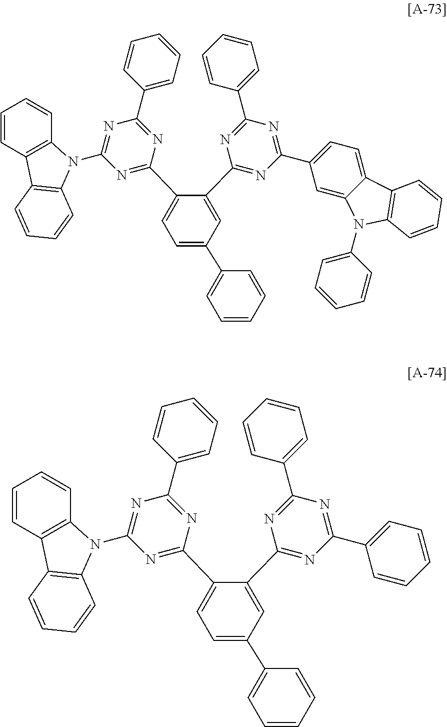 [SMILES: c1ccc(-c2ccc(-c3nc(-c4ccccc4)nc(-n4c5ccccc5c5ccccc54)n3)c(-c3nc(-c4ccccc4)nc(-c4ccc5c6ccccc6n(-c6ccccc6)c5c4)n3)c2)cc1 ; c1ccc(-c2ccc(-c3nc(-c4ccccc4)nc(-n4c5ccccc5c5ccccc54)n3)c(-c3nc(-c4ccccc4)nc(-c4ccccc4)n3)c2)cc1]

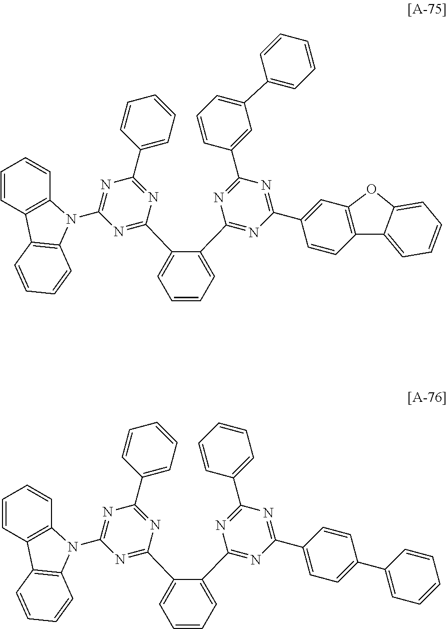 [SMILES: c1ccc(-c2cccc(-c3nc(-c4ccc5c(c4)oc4ccccc45)nc(-c4ccccc4-c4nc(-c5ccccc5)nc(-n5c6ccccc6c6ccccc65)n4)n3)c2)cc1 ; c1ccc(-c2ccc(-c3nc(-c4ccccc4)nc(-c4ccccc4-c4nc(-c5ccccc5)nc(-n5c6ccccc6c6ccccc65)n4)n3)cc2)cc1]

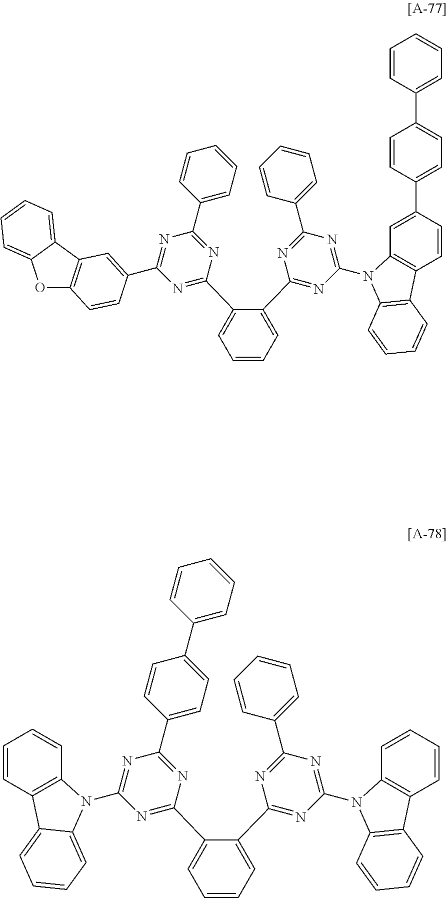 [SMILES: c1ccc(-c2ccc(-c3ccc4c5ccccc5n(-c5nc(-c6ccccc6)nc(-c6ccccc6-c6nc(-c7ccccc7)nc(-c7ccc8oc9ccccc9c8c7)n6)n5)c4c3)cc2)cc1 ; c1ccc(-c2ccc(-c3nc(-c4ccccc4-c4nc(-c5ccccc5)nc(-n5c6ccccc6c6ccccc65)n4)nc(-n4c5ccccc5c5ccccc54)n3)cc2)cc1]

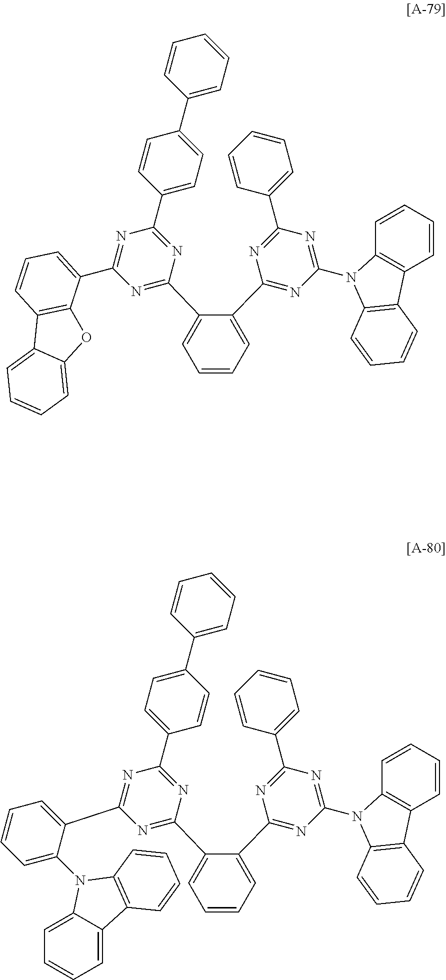 [SMILES: c1ccc(-c2ccc(-c3nc(-c4ccccc4-c4nc(-c5ccccc5)nc(-n5c6ccccc6c6ccccc65)n4)nc(-c4cccc5c4oc4ccccc45)n3)cc2)cc1 ; c1ccc(-c2ccc(-c3nc(-c4ccccc4-c4nc(-c5ccccc5)nc(-n5c6ccccc6c6ccccc65)n4)nc(-c4ccccc4-n4c5ccccc5c5ccccc54)n3)cc2)cc1]

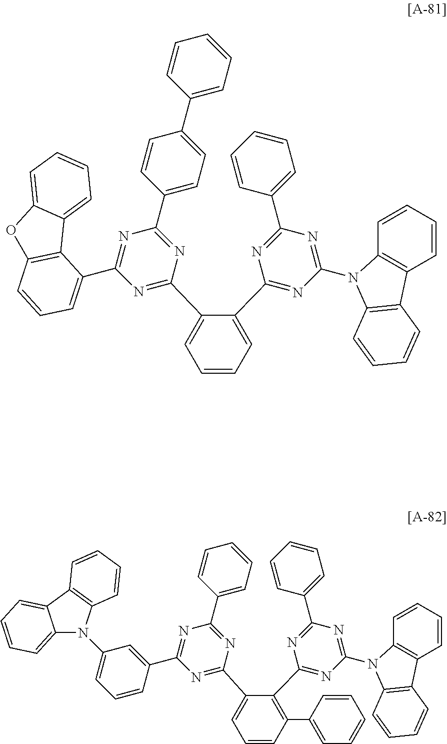 [SMILES: c1ccc(-c2ccc(-c3nc(-c4ccccc4-c4nc(-c5ccccc5)nc(-n5c6ccccc6c6ccccc65)n4)nc(-c4cccc5oc6ccccc6c45)n3)cc2)cc1 ; c1ccc(-c2nc(-c3cccc(-n4c5ccccc5c5ccccc54)c3)nc(-c3cccc(-c4ccccc4)c3-c3nc(-c4ccccc4)nc(-n4c5ccccc5c5ccccc54)n3)n2)cc1]

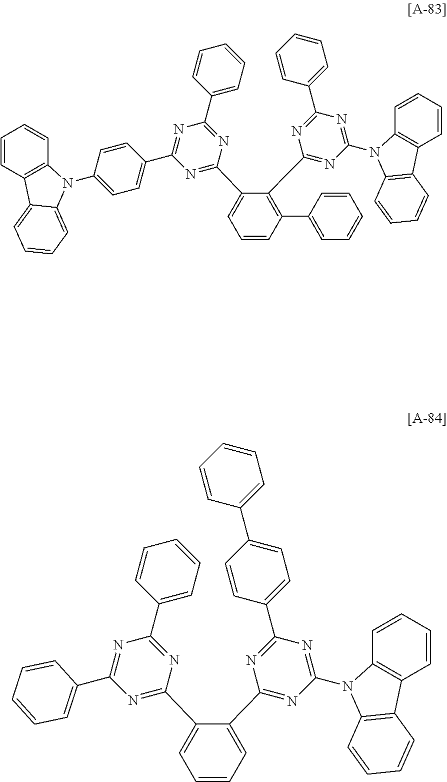 [SMILES: c1ccc(-c2nc(-c3ccc(-n4c5ccccc5c5ccccc54)cc3)nc(-c3cccc(-c4ccccc4)c3-c3nc(-c4ccccc4)nc(-n4c5ccccc5c5ccccc54)n3)n2)cc1 ; c1ccc(-c2ccc(-c3nc(-c4ccccc4-c4nc(-c5ccccc5)nc(-c5ccccc5)n4)nc(-n4c5ccccc5c5ccccc54)n3)cc2)cc1]

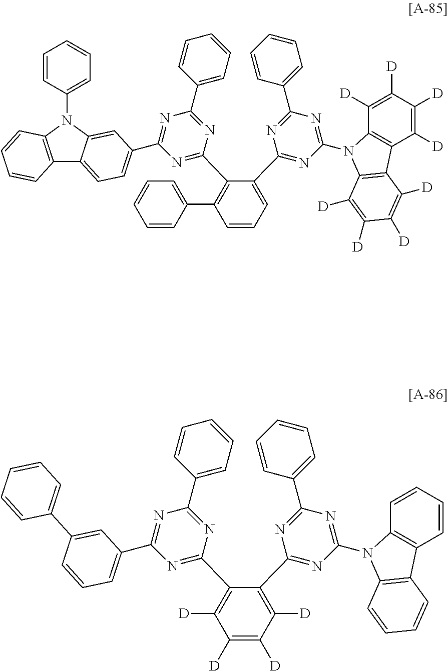 [SMILES: [2H]c1c([2H])c([2H])c2c(c1[2H])c1c([2H])c([2H])c([2H])c([2H])c1n2-c1nc(-c2ccccc2)nc(-c2cccc(-c3ccccc3)c2-c2nc(-c3ccccc3)nc(-c3ccc4c5ccccc5n(-c5ccccc5)c4c3)n2)n1 ; [2H]c1c([2H])c([2H])c(-c2nc(-c3ccccc3)nc(-n3c4ccccc4c4ccccc43)n2)c(-c2nc(-c3ccccc3)nc(-c3cccc(-c4ccccc4)c3)n2)c1[2H]]

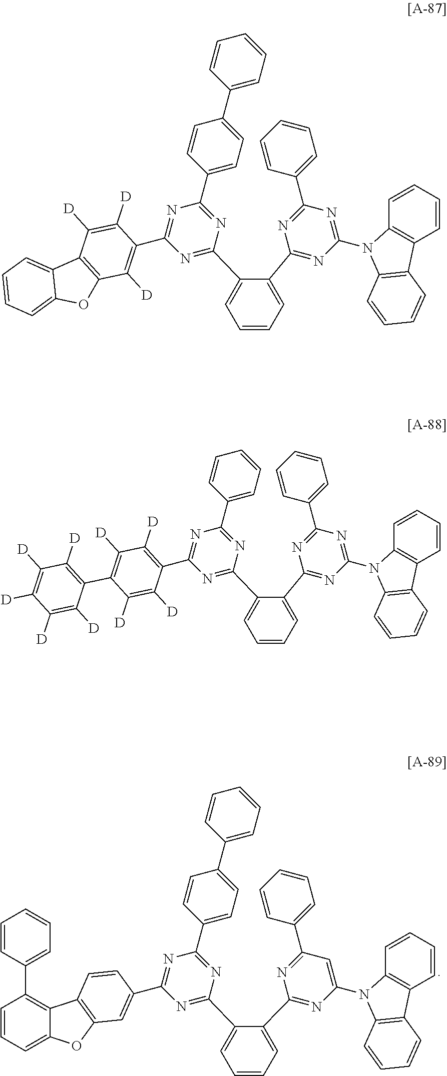 [SMILES: [2H]c1c(-c2nc(-c3ccc(-c4ccccc4)cc3)nc(-c3ccccc3-c3nc(-c4ccccc4)nc(-n4c5ccccc5c5ccccc54)n3)n2)c([2H])c2oc3ccccc3c2c1[2H] ; [2H]c1c([2H])c([2H])c(-c2c([2H])c([2H])c(-c3nc(-c4ccccc4)nc(-c4ccccc4-c4nc(-c5ccccc5)nc(-n5c6ccccc6c6ccccc65)n4)n3)c([2H])c2[2H])c([2H])c1[2H] ; c1ccc(-c2ccc(-c3nc(-c4ccc5c(c4)oc4cccc(-c6ccccc6)c45)nc(-c4ccccc4-c4nc(-c5ccccc5)cc(-n5c6ccccc6c6ccccc65)n4)n3)cc2)cc1]

8. A composition for an organic optoelectronic device, the composition comprising
a first compound and a second compound,
wherein the first compound is the compound for an organic optoelectronic device as claimed in claim 1, and
the second compound is represented by Chemical Formula 2 or a combination of Chemical Formula 3 and Chemical Formula 4: 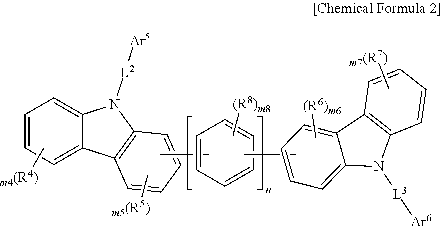
wherein, in Chemical Formula 2,
R⁴ to R⁸ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁵ and Ar⁶ are each independently a substituted or unsubstituted C6 to C20 aryl group or a substituted or unsubstituted C2 to C30 heterocyclic group,
L² and L³ are each independently a single bond or a substituted or unsubstituted C6 to C20 arylene group,
m4, m7, and m8 are each independently an integer of 1 to 4,
m5 and m6 are each independently an integer of 1 to 3, and
n is an integer of 0 to 2; 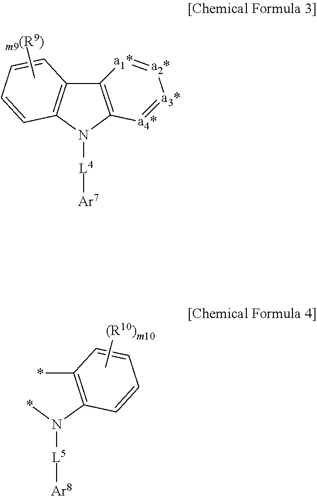
wherein, in Chemical Formula 3 and Chemical Formula 4,
two adjacent ones of a₁* to a₄* in Chemical Formula 3 are linking carbons linked to * of Chemical Formula 4, the remaining two of a1* to a4* of Chemical Formula 3, not linked to * of Chemical Formula 4, are C-L^{a}-R^{a},
L^{a}, L⁴, and L⁵ are each independently a single bond or a substituted or unsubstituted C6 to C20 arylene group,
R^{a}, R⁹, and R¹⁰ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁷ and Ar⁸ are each independently a substituted or unsubstituted C6 to C20 aryl group or a substituted or unsubstituted C2 to C30 heterocyclic group, and
m9 and m10 are each independently an integer of 1 to 4.

9. The composition for an organic optoelectronic device as claimed in claim 8, wherein;
the second compound is represented by Chemical Formula 2, and
Chemical Formula 2 is represented by Chemical Formula 2-8: 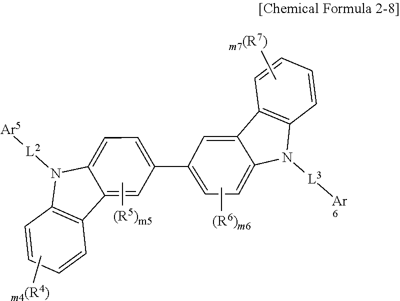
in Chemical Formula 2-8,
R⁴ to R⁷ are each independently hydrogen, deuterium, or a substituted or unsubstituted C6 to C12 aryl group,
m4 and m7 are each independently an integer of 1 to 4,
m5 and m6 are each independently an integer of 1 to 3, and
moieties *-L²-Ar⁵ and *-L³-Ar⁶ are each independently a moiety of Group I: 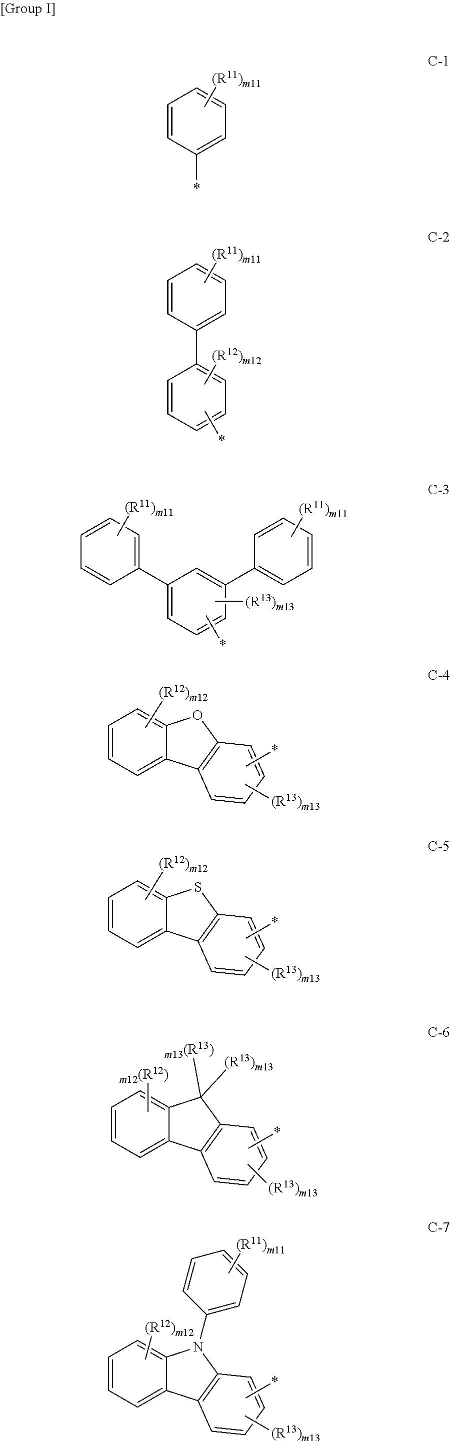 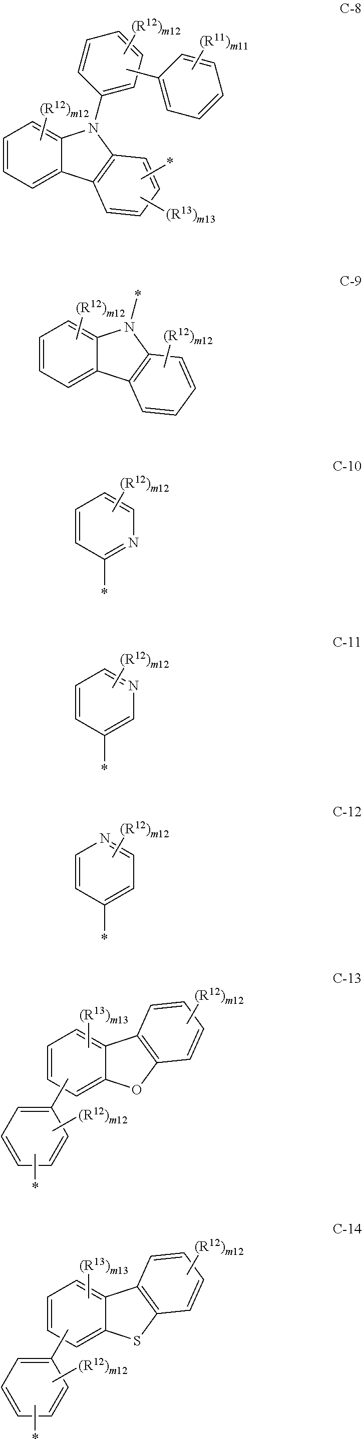 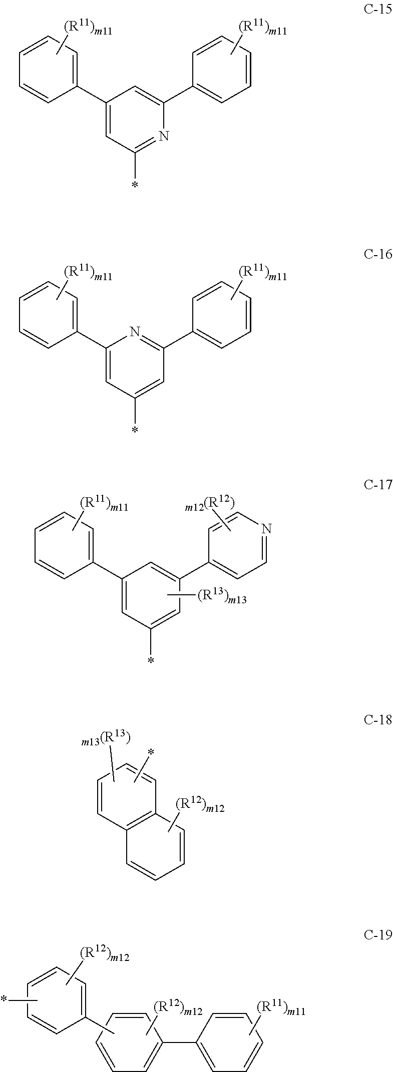 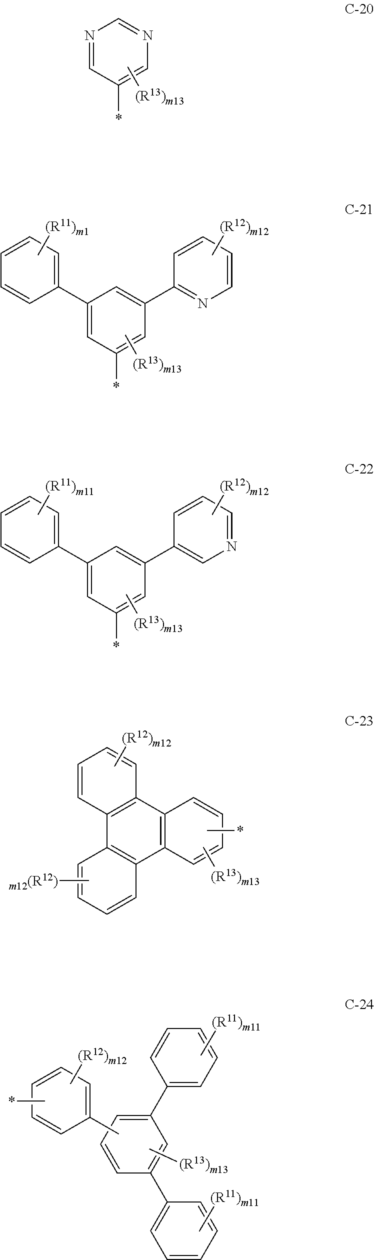 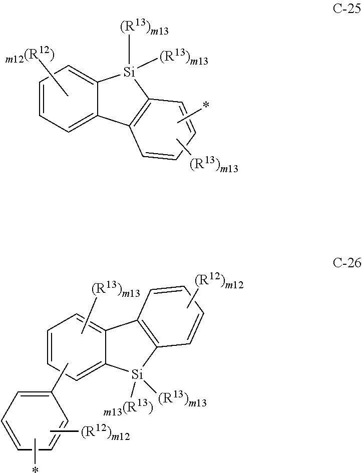
in Group I,
R¹¹ to R¹³ are each independently hydrogen, deuterium, a substituted or unsubstituted C1 to C4 alkyl group, a substituted or unsubstituted C6 to C18 aryl group, or a substituted or unsubstituted C2 to C30 heteroaryl group,
m11 is an integer of 1 to 5,
m12 is an integer of 1 to 4,
m13 is an integer of 1 to 3, and
* is a linking point.

10. The composition for an organic optoelectronic device as claimed in claim 8, wherein:
the second compound is represented by a combination of Chemical Formula 3 and Chemical Formula 4, and
the combination of Chemical Formula 3 and Chemical Formula 4 is represented by Chemical Formula 3C: 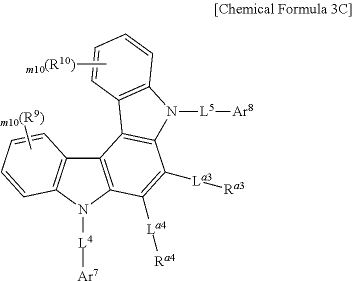
in Chemical Formula 3C,
L^{a3} and L^{a4} are a single bond,
L⁴ and L⁵ are each independently a single bond or a substituted or unsubstituted C6 to C12 arylene group,
R⁹ and R¹⁰ are each independently hydrogen, deuterium, or C6 to C12 aryl group,
Ar⁷ and Ar⁸ are each independently a substituted or unsubstituted phenyl group, a substituted or unsubstituted naphthyl group, or a substituted or unsubstituted biphenyl group, and
m9 and m10 are each independently an integer of 1 to 4.

11. An organic optoelectronic device, comprising:
an anode and a cathode facing each other, and
at least one organic layer between the anode and the cathode,
wherein the at least one organic layer includes the compound for the organic optoelectronic device as claimed in claim 1.

12. The organic optoelectronic device as claimed in claim 11, wherein:
the at least one organic layer includes a light emitting layer, and
the light emitting layer includes the compound for an organic optoelectronic device.

13. A display device comprising the organic optoelectronic device as claimed in claim 11.

14. An organic optoelectronic device, comprising:
an anode and a cathode facing each other, and
at least one organic layer between the anode and the cathode,
wherein the at least one organic layer includes the composition for the organic optoelectronic device as claimed in claim 8.

15. The organic optoelectronic device as claimed in claim 14, wherein:
the at least one organic layer includes a light emitting layer, and
the light emitting layer includes the composition for an organic optoelectronic device.

16. A display device comprising the organic optoelectronic device as claimed in claim 14.
